(19) United States
(12) Patent Application Publication
(10) Pub. No.: US 2025/0281181 A1
(43) Pub. Date: Sep. 11, 2025

(54) ENDOSCOPIC MAGNETIC ANASTOMOSIS SYSTEM, DEVICES AND METHOD

(71) Applicant: iEMIS (HK) Limited, Hong Kong SAR (CN)

(72) Inventors: Chung Kwong YEUNG, Hong Kong SAR (CN); Wing Fai LAM, Hong Kong SAR (CN); Hon Shing CHAN, Hong Kong SAR (CN); Biji SREEDHAR, Hong Kong SAR (CN)

(21) Appl. No.: 19/217,247

(22) Filed: May 23, 2025

Related U.S. Application Data
(63) Continuation-in-part of application No. 18/015,172, filed on Jan. 9, 2023.

(51) Int. Cl. A61B 17/11; A61B 1/00; A61B 1/018; A61B 1/05; A61B 17/00; A61B 90/00

(52) U.S. Cl. CPC A61B 17/1114; A61B 1/00018; A61B 1/00097; A61B 1/00119; A61B 1/00124; A61B 1/00128; A61B 1/00137; A61B 1/00147; A61B 1/018; A61B 1/05; A61B 17/00234; A61B 90/03; A61B 90/06; A61B 90/08; A61B 2017/00039; A61B 2017/00238; A61B 2017/00296; A61B 2017/00358; A61B 2017/00367; A61B 2017/00557; A61B 2017/00876; A61B 2017/1117; A61B 2090/034; A61B 2090/0807; A61B 2217/005

(57) ABSTRACT

An endoscopic magnetic anastomosis system, devices and method for forming an anastomosis. The endoscopic magnetic anastomosis system includes an endoscopic assembly, a video processor console, a magnet detector, and a magnetic navigation console. The endoscopic assembly includes an endoscope, an adjustable snare mechanism, an outer sheath, and a magnetic implant assembly. The adjustable snare mechanism includes a snare assembly. The snare assembly passes through a snare channel in the endoscope and includes a snare catheter and a snare wire passing through the snare catheter. The snare is used to selectively tighten and release the magnetic implant assembly disposed at one end of the endoscope.

### CROSS-REFERENCE TO RELATED APPLICATIONS

This application is a continuation-in-part of U.S. application Ser. No. 18/015,172 filed on Jan. 9, 2023, which is a 371 U.S. national phase application of the PCT Application No. PCT/CN2020/100662 filed on Jul. 7, 2020. The above-identified applications are hereby incorporated herein by reference in their entirety.

### TECHNICAL FIELD

The present disclosure relates generally to a robotic system, devices, and method for forming an anastomosis, and more particularly, to an endoscopic magnetic anastomosis system and devices for forming an anastomosis.

### BACKGROUND

Diabetes is emerging as a primary cause of mortality, morbidity, disability, and discrimination in health care, education and employment. The International Diabetes Federation (IDF) estimates nearly half a billion people worldwide are presently living with diabetes. It is estimated that by 2045, the global prevalence will increase by 48%, swelling to an estimated 693 million individuals (aged 18-99 years) afflicted.

The socioeconomic burden associated with these conditions is immense, most of which can be attributed to Diabetes. Hyperglycemia, a high level of blood glucose, is the hallmark of diabetes. In Type 2 Diabetes (T2DM), hyperglycemia results from a varying combination of insulin resistance and inadequate insulin production. Chronic hyperglycaemia can cause damage to various organs, leading to the development of disabling and life-threatening complications such as cardiovascular disease, neuropathy, nephropathy, and eye disease leading to retinopathy and blindness. These complications contribute to more frequent hospitalization, increased medical care costs and lower quality of life, and often results in early death.

### SUMMARY

Present example embodiments relate generally to and/or comprise systems, subsystems, processors, devices, logic, and methods for addressing conventional problems, including those described above.

In an exemplary embodiment, an endoscopic anastomosis system is described. The endoscopic anastomosis system includes a first main body assembly. The first main body assembly is an elongated body having a first end and a second end. At least a portion of the second end of the first main body assembly is controllable to bend in a plurality of directions. The endoscopic anastomosis system includes a head assembly. The head assembly includes a first end and a second end. The head assembly includes a head assembly body and a first expandable member. The head assembly body includes a first region and a second region. The first region includes a first end, a second end, and a midsection between the first and second ends of the first region. The first end of the first region is secured to the second end of the first main body assembly. The second end of the first region includes a first section and a second section. The first section is secured to a first end of a second region of the head assembly. The second section includes a second main body assembly opening. The second region includes the first end and a second end. The second end of the second region includes a first pressure port. The first pressure port is configured to apply a negative pressure. The first expandable member is secured to at least a portion of the midsection of the first region of the head assembly body. The first expandable member is configured to expand radially away from the first region of the head assembly body. The endoscopic anastomosis system includes a second main body assembly. The second main body assembly is an elongated body having a first end and a second end. At least a portion of the second end of the second main body assembly is provided in and moveable through the second main body assembly opening of the second section of the first region of the head assembly body. The second main body assembly includes a second expandable member and a second pressure port. The second expandable member is secured to a portion of the second end of the second main body assembly. The second expandable member is configured to expand radially away from the second main body assembly. The second pressure port is configured to apply a negative pressure. The endoscopic anastomosis system includes a magnetic implant assembly. The magnetic implant assembly includes a magnetic body. The magnetic body is formed as a substantially flat body. The magnetic body includes a front wall, a rear wall opposite to the front wall, and an exterior circumferential sidewall. The front wall has a circular shape with a central axis. The exterior circumferential sidewall is formed around the magnetic body. The exterior circumferential sidewall defines a thickness of the magnetic body. The exterior circumferential sidewall is formed at a first radius from the central axis. The endoscopic anastomosis system includes a securing assembly. The securing assembly is secured to the second end of the second main body assembly. The securing assembly is actuatable between a locked configuration and an unlocked configuration. The locked configuration is a configuration in which the magnetic implant assembly is secured to the securing assembly. The unlocked configuration is a configuration in which the magnetic implant assembly is not secured to the securing assembly.

In another exemplary embodiment, a catheter system for an endoscopic anastomosis system is described. The endoscopic anastomosis system includes a main body and a head assembly. The head assembly is secured to a distal end of the main body. The head assembly includes a head assembly body, a first expandable member, and a first pressure port. The head assembly body includes a first region and a second region. The first region includes a first end and a second end. The first end of the first region includes a first section and a second section. The first section of the first region is secured to the second region. The second section of the first region includes a catheter body opening for at least a portion of the catheter assembly to be provided in and moveable through. The first expandable member is secured to a portion of the first region between the first and second end of the first region. The first pressure port is provided in the second region. The catheter assembly includes a catheter body, a magnetic implant assembly, and a securing assembly. The catheter body is an elongated body. The catheter body includes a first end and a second end. At least a portion of the second end of the catheter body is provided in and moveable through the catheter body opening of the first region of the head assembly body. The catheter body includes a second expandable member and a second pressure port. The second expandable member is secured to a portion of the second end of the catheter body. The second expandable member is configured to expand radially away from the catheter body. The second pressure port is configured to apply a negative pressure. The magnetic implant assembly includes a magnetic body. The magnetic body is formed as a substantially flat body. The magnetic body includes a front wall, a rear wall opposite to the front wall, and an exterior circumferential sidewall. The front wall has a circular shape with a central axis. The exterior circumferential sidewall is formed around the magnetic body. The exterior circumferential sidewall defines a thickness of the magnetic body. The exterior circumferential sidewall is formed at a first radius from the central axis. The securing assembly is secured to the second end of the catheter body. The securing assembly is actuatable between a locked configuration and an unlocked configuration. The locked configuration is a configuration in which the magnetic implant assembly is secured to the securing assembly. The unlocked configuration is a configuration in which the magnetic implant assembly is not secured to the securing assembly.

In another exemplary embodiment, magnetic implant assembly for an endoscopic anastomosis system is described. The endoscopic anastomosis system includes a main body and one or more protruding portions formed on the main body. The main body includes a first end and a second end. The main body includes a securing assembly secured to the second end of the main body. The securing assembly is actuatable between a locked configuration and an unlocked configuration. The locked configuration is a configuration in which the magnetic implant assembly is secured to the securing assembly. The unlocked configuration is a configuration in which the magnetic assembly is not secured to the securing assembly. The magnetic implant assembly comprises a magnetic body. The magnetic body is formed as a substantially flat body. The magnetic body includes a front wall, rear wall opposite to the front wall, and an exterior circumferential sidewall formed around the magnetic body. The front wall includes a circular shape with a central axis. The exterior circumferential sidewall is formed around the magnetic body. The exterior circumferential sidewall defines a thickness of the magnetic body. The exterior circumferential sidewall is formed at a first radius from the central axis. The one or more protruding portions is formed on the front wall of the magnetic body. The one or more protruding portions includes a first ring shaped protrusion formed on the front wall of the magnetic body. The first ring shaped protrusion is centrally aligned with the central axis. The first ring shaped protrusion includes a first exterior radius from the central axis and a first interior radius from the central axis. The first exterior radius of the first ring shaped protrusion is less than the first radius. Alternatively or in addition, the first exterior radius of the first ring shaped protrusion is equal to the first radius and the magnetic implant assembly further includes an exterior ring shaped body. The exterior ring shaped body is formed around and fixedly secured to the exterior circumferential sidewall of the magnetic body. The exterior ring shaped body includes a front exterior ring shaped portion adjacent to the front wall of the magnetic body and a rear exterior ring shaped portion adjacent to the rear wall of the magnetic body. At least a portion of the front exterior ring shaped body is formed using a material other than a ferromagnetic or magnetic material.

In another exemplary embodiment, there is provided an endoscopic magnetic anastomosis system including at least one endoscopic assembly including: an endoscope having a first end and an opposite second end, the endoscope including a snare channel extending from the first end to the second end and a magnetic implant assembly disposed at the second end; and an adjustable snare mechanism including: a snare assembly passing through the snare channel and including a snare catheter and a snare wire passing through the snare catheter for selectively tightening and releasing the magnetic implant assembly.

In another exemplary embodiment, there is provided an outer sheath including: a body tube having a first end and an opposite second end; and a tube-locking mechanism mounted at the first end of the body tube, wherein the body tube further has an endoscopic channel extending from the first end of the body tube to the second end of the body tube for passage of the endoscope, the tube-locking mechanism is configured to be able to be in locking fit with the endoscope passing through the endoscopic channel to secure the endoscope with the body tube, and to disengage from being in locking fit with the endoscope passing through the endoscopic channel to enable the endoscope to slide and rotate relative to the body tube.

In another exemplary embodiment, there is provided a magnet detector for detecting a position of a magnet, the magnet detector including: a detector body; a first printed circuit board provided on a first end of the detector body and provided with a first group of magnetometers; a second printed circuit board provided on a second end of the detector body opposite to the first end, and provided with a second group of magnetometers; and a processing unit configured to be able to receive measurement data from the first group of magnetometers and the second group of magnetometers and to determine a position of the magnet based on the received data.

In another exemplary embodiment, there is provided a magnetic navigation console for driving a magnet to move under action of a magnetic field, the magnetic navigation console including: a mounting bracket including a movable first mounting arm and a movable second mounting arm; a first magnetic actuator mounted to the first mounting arm; and a second magnetic actuator mounted to the second mounting arm, the first magnetic actuator and the second magnetic actuator are configured to be able to move to overlap in a vertical direction.

Other features and advantages of the present application will be set forth in the following specification.

### BRIEF DESCRIPTION OF THE DRAWINGS

For a more complete understanding of the present disclosure, example embodiments, and their advantages, reference is now made to the following description taken in conjunction with the accompanying figures, in which like reference numbers indicate like features, and:

FIG. 1A is a perspective view illustration of an example embodiment of an endoscopic anastomosis system;

FIG. 1B is a side view illustration of an example embodiment of an endoscopic anastomosis system;

FIG. 1C is another side view illustration of an example embodiment of an endoscopic anastomosis system;

FIG. 2 is a perspective view illustration of an example embodiment of some of the elements of an endoscopic anastomosis system separated from one another;

FIG. 3A is a side view illustration of an example embodiment of a first main body assembly;

FIG. 3B is a side view illustration of an example embodiment of a first main body assembly and the first bendable section configured to bend;

FIG. 3C is another side view illustration of an example embodiment of a first main body assembly and the first bendable section configured to bend;

FIG. 4A is a perspective view illustration of an example embodiment of a head assembly with a magnetic implant assembly;

FIG. 4B is a perspective view illustration of an example embodiment of a head assembly without a magnetic implant assembly;

FIG. 4C is a side view illustration of an example embodiment of a head assembly with first expandable member not expanded;

FIG. 4D is a side view illustration of an example embodiment of a head assembly with first expandable member expanded;

FIG. 4E is a side view illustration of an example embodiment of a second main body assembly extended outward away from a head assembly;

FIG. 4F is a side view illustration of an example embodiment of a second main body assembly extended outward away from a head assembly, and a first expandable member configured to expand;

FIG. 4G is a side view illustration of an example embodiment of a second main body assembly extended outward away from a head assembly, and a second bendable section configured to bend;

FIG. 4H is a side view illustration of an example embodiment of a second main body assembly extended outward away from a head assembly, a second bendable section configured to bend, and a second expandable member configured to expand;

FIG. 4I is a side view illustration of an example embodiment of a second main body assembly extended outward away from a head assembly and rotated so as to change an orientation of a magnetic implant assembly;

FIG. 5A is a perspective view illustration of an example embodiment of a magnetic implant assembly secured to a second main body assembly via a securing assembly;

FIG. 5B is a cross-sectional view illustration of an example embodiment of a magnetic implant assembly, a securing assembly, and a second main body assembly;

FIG. 5C is a perspective view illustration of another example embodiment of a magnetic implant assembly secured to a second main body assembly via a securing assembly;

FIG. 5D is a cross-sectional view illustration of another example embodiment of a magnetic implant assembly, a securing assembly, and a second main body assembly;

FIG. 5E is a perspective view illustration of another example embodiment of a magnetic implant assembly secured to a second main body assembly via a securing assembly;

FIG. 5F is a cross-sectional view illustration of another example embodiment of a magnetic implant assembly, a securing assembly, and a second main body assembly;

FIG. 5G is a perspective view illustration of another example embodiment of a magnetic implant assembly secured to a second main body assembly via a securing assembly;

FIG. 5H is a cross-sectional view illustration of another example embodiment of a magnetic implant assembly, a securing assembly, and a second main body assembly;

FIG. 6A is a cross-sectional view illustration of an example embodiment of a magnetic implant assembly and a second magnetic implant assembly;

FIG. 6B is a cross-sectional view illustration of an example embodiment of a magnetic implant assembly magnetically coupled to a second magnetic implant assembly;

FIG. 6C is a top view illustration of an example embodiment of a front wall of a magnetic implant assembly;

FIG. 6D is a top view illustration of another example embodiment of a front wall of a magnetic implant assembly;

FIG. 7A is a cross-sectional view illustration of another example embodiment of a magnetic implant assembly and a second magnetic implant assembly;

FIG. 7B is a cross-sectional view illustration of another example embodiment of a magnetic implant assembly magnetically coupled to a second magnetic implant assembly;

FIG. 7C is a top view illustration of an example embodiment of a front wall of a magnetic implant assembly;

FIG. 7D is a top view illustration of another example embodiment of a front wall of a magnetic implant assembly;

FIGS. 8A-S are illustrations of an example embodiment of a method of delivering a magnetic implant assembly rectally;

FIGS. 9A-I are illustrations of an example embodiment of a method of delivering a magnetic implant assembly orally;

FIG. 10 is a schematic structural diagram illustrating the usage state of an endoscopic magnetic anastomosis system according to an embodiment of the present application;

FIG. 11 is a schematic diagram of a structure of an endoscopic magnetic anastomosis system according to an embodiment of the present application;

FIG. 12A is a schematic structural diagram illustrating a process of using an adjustable snare mechanism of an endoscope magnetic anastomosis system according to an embodiment of the present application;

FIGS. 12B-12D are schematic cross-sectional structural diagrams illustrating a process of using an adjustable snare mechanism of an endoscopic magnetic anastomosis system according to an embodiment of the present application;

FIG. 13A is a schematic diagram of a structure of an outer sheath and an endoscope of an endoscope magnetic anastomosis system according to an embodiment of the present application, wherein a tube-locking mechanism is in a locked state;

FIG. 13B is a schematic diagram of a structure of an outer sheath and an endoscope of an endoscope magnetic anastomosis system according to an embodiment of the present application, wherein the tube-locking mechanism is in an unlocked state;

FIG. 13C is a schematic diagram of a structure of an outer sheath of an endoscopic magnetic anastomosis system according to an embodiment of the present application;

FIG. 13D is a schematic diagram of a partial structure of a body tube of an outer sheath of an endoscopic magnetic anastomosis system according to an embodiment of the present application;

FIG. 13E is a schematic diagram of a structure of a tube-locking mechanism of an outer sheath of an endoscope magnetic anastomosis system according to an embodiment of the present application;

FIG. 13F is a partial cross-sectional schematic view of a structure of a distal end of an assembly structure of an outer sheath and an endoscope of an endoscope magnetic anastomosis system according to an embodiment of the present application;

FIG. 13G is a partial cross-sectional schematic view of a structure of a proximal end of an assembly structure of an outer sheath and an endoscope of an endoscope magnetic anastomosis system according to an embodiment of the present application;

FIG. 13H is a schematic cross-sectional structural diagram of an external pressure source of an outer sheath of an endoscopic magnetic anastomosis system according to an embodiment of the present application in different working states;

FIG. 13I is a schematic cross-sectional structural diagram of a body tube of an outer sheath of an endoscopic magnetic anastomosis system according to an embodiment of the present application;

FIG. 14A is a schematic diagram of a three-dimensional structure of a head assembly of an endoscope of an endoscope magnetic anastomosis system according to an embodiment of the present application;

FIG. 14B is a schematic view of a front view structure of a head assembly of an endoscope of an endoscope magnetic anastomosis system according to an embodiment of the present application;

FIG. 14C is a schematic diagram of a left side structure of a head assembly of an endoscope of an endoscope magnetic anastomosis system according to an embodiment of the present application;

FIG. 14D is a schematic diagram of an assembly structure of a head assembly and a magnetic implant assembly of an endoscope of an endoscope magnetic anastomosis system according to an embodiment of the present application;

FIG. 14E is a schematic diagram of a structure of a head assembly of an endoscope of an endoscopic magnetic anastomosis system in some cases;

FIG. 14F is a schematic diagram of an assembly structure of a head assembly and a magnetic implant assembly of an endoscope of an endoscopic magnetic anastomosis system in some cases;

FIG. 15A is a schematic diagram of an assembly structure of a video processor and a cable of an endoscope magnetic anastomosis system according to an embodiment of the present application;

FIG. 15B is a schematic structural diagram illustrating an assembly process of a video processor and a cable of an endoscope magnetic anastomosis system according to an embodiment of the present application;

FIG. 15C is a schematic diagram of a three-dimensional structure of a main connector socket of a video processor of an endoscope magnetic anastomosis system according to an embodiment of the present application;

FIG. 15D is a schematic cross-sectional structural diagram of a main connector socket of a video processor of an endoscope magnetic anastomosis system according to an embodiment of the present application, wherein the main connector socket is in an initial state;

FIG. 15E is a schematic cross-sectional structural diagram of a main connector socket of a video processor of an endoscope magnetic anastomosis system according to an embodiment of the present application, wherein the main connector socket is in a locked state;

FIG. 15F is a schematic cross-sectional structural diagram of a main connector socket of a video processor of an endoscope magnetic anastomosis system according to an embodiment of the present application;

FIG. 15G is a schematic diagram of a structure of a main connector of a cable of an endoscope magnetic anastomosis system according to an embodiment of the present application;

FIG. 15H is a schematic cross-sectional structural diagram of a main connector socket of a video processor and a main connector of a cable of an endoscope magnetic anastomosis system according to an embodiment of the present application before plug-in connection;

FIG. 15I is a schematic cross-sectional structural diagram of a main connector socket of a video processor and a main connector of a cable of an endoscope magnetic anastomosis system according to an embodiment of the present application during plug-in connection;

FIG. 16A is a schematic diagram of a three-dimensional structure of a magnetic implant assembly of an endoscope of an endoscope magnetic anastomosis system according to an embodiment of the present application;

FIG. 16B is another schematic diagram of a three-dimensional structure of a magnetic implant assembly of an endoscope of an endoscope magnetic anastomosis system according to an embodiment of the present application;

FIG. 16C is a schematic cross-sectional structural diagram of a magnetic implant assembly of an endoscope of an endoscope magnetic anastomosis system according to an embodiment of the present application;

FIG. 16D is a schematic structural diagram illustrating an cooperative use of magnetic implant assemblies of two endoscopes of an endoscopic magnetic anastomosis system according to an embodiment of the present application;

FIG. 16E is a schematic cross-sectional structural diagram of a magnetic implant assembly of an endoscope of an endoscopic magnetic anastomosis system in some cases;

FIG. 16F is a schematic diagram of a magnetic field interaction between two magnetic implant assemblies according to an embodiment of the present application;

FIG. 16G is a schematic diagram of a magnetic field interaction between two magnetic implant assemblies in some cases;

FIG. 16H is a schematic diagram of a relationship between the magnetic field force and distance between two magnetic implant assemblies according to an embodiment of the present application;

FIG. 16I is a schematic diagram of a relationship between the magnetic field force and distance between two magnetic implant assemblies in some cases;

FIG. 17A is a schematic diagram of a structure of a magnet detector of an endoscopic magnetic anastomosis system according to an embodiment of the present application;

FIG. 17B is a schematic view of an exploded structure of a magnet detector of an endoscopic magnetic anastomosis system according to an embodiment of the present application;

FIG. 17C is a top view schematic diagram of a structure of the first printed circuit board of the magnet detector of an endoscopic magnetic anastomosis system according to an embodiment of the present application;

FIG. 17D is a bottom view schematic diagram of a structure of the first printed circuit board of the magnet detector of an endoscopic magnetic anastomosis system according to an embodiment of the present application;

FIG. 17E is a top view schematic diagram of a structure of the second printed circuit board of the magnet detector of an endoscopic magnetic anastomosis system according to an embodiment of the present application;

FIG. 17F is a bottom view schematic diagram of a structure of the second printed circuit board of the magnet detector of an endoscopic magnetic anastomosis system according to an embodiment of the present application;

FIG. 18A is a schematic diagram of a structure of a magnet detector of an endoscopic magnetic anastomosis system according to another embodiment of the present application;

FIG. 18B is a schematic diagram of the working principle of a magnet detector of an endoscopic magnetic anastomosis system according to another embodiment of the present application;

FIG. 19A is a schematic diagram of a three-dimensional structure of a magnetic navigation console of an endoscopic magnetic anastomosis system according to an embodiment of the present application;

FIG. 19B is a schematic diagram of a top view structure of a magnetic navigation console of an endoscopic magnetic anastomosis system according to an embodiment of the present application;

FIG. 19C is a schematic structural diagram illustrating a working process of a push rod mechanism of a magnetic navigation console of an endoscope magnetic anastomosis system according to an embodiment of the present application;

FIG. 19D is a schematic structural diagram illustrating a push rod mechanism of a magnetic navigation console of an endoscope magnetic anastomosis system according to an embodiment of the present application in different usage states;

FIG. 19E is a schematic diagram of a structure of a first magnetic actuator of a magnetic navigation console of an endoscopic magnetic anastomosis system according to an embodiment of the present application; and

FIG. 19F is a schematic diagram illustrating a magnetic field effect of a first magnetic actuator of a magnetic navigation console on a magnetic implant assembly of an endoscopic magnetic anastomosis system according to an embodiment of the present application.

The drawings are used for providing an understanding of technical solutions of the present application, and constitute a part of the specification. They are used together with embodiments of the present application to explain the technical solutions of the present application, and do not constitute a restriction on the technical solutions of the present application.

Although similar reference numbers may be used to refer to similar elements in the figures for convenience, it can be appreciated that each of the various example embodiments may be considered to be distinct variations.

Example embodiments will now be described with reference to the accompanying figures, which form a part of the present disclosure and which illustrate example embodiments which may be practiced. As used in the present disclosure and the appended claims, the terms “embodiment,” “example embodiment,” “exemplary embodiment,” and “present embodiment” do not necessarily refer to a single embodiment, although they may, and various example embodiments may be readily combined and/or interchanged without departing from the scope or spirit of example embodiments. Furthermore, the terminology as used in the present disclosure and the appended claims is for the purpose of describing example embodiments only and is not intended to be limitations. In this respect, as used in the present disclosure and the appended claims, the term “in” may include “in” and “on,” and the terms “a,” “an,” and “the” may include singular and plural references. Furthermore, as used in the present disclosure and the appended claims, the term “by” may also mean “from,” depending on the context. Furthermore, as used in the present disclosure and the appended claims, the term “if” may also mean “when” or “upon,” depending on the context. Furthermore, as used in the present disclosure and appended claims, the words “and/or” may refer to and encompass any or all possible combinations of one or more of the associated listed items.

### DETAILED DESCRIPTION

Along with obesity, diabetes is emerging as a primary cause of mortality, morbidity, disability, and discrimination in health care, education and employment. The International Diabetes Federation (IDF) estimates that at present, nearly half a billion people worldwide are living with diabetes, of which low income and middle income countries carry close to 80% of the burden. Moreover, worldwide growth in prevalence is projected to only further worsen over the coming years. It is estimated that by 2045, the global prevalence will increase by 48%, swelling to an estimated 693 million individuals (aged 18-99 years) afflicted. Likewise, there has been a closely paralleled marked increase in obesity. The World Health Organization (WHO) estimates that in 2016, more than 1.9 billion adults, 18 years and older, were overweight. Of these, over 650 million were obese.

The socioeconomic burden associated with these conditions is immense, most of which can be attributed to Diabetes. Hyperglycemia, a high level of blood glucose, is the hallmark of diabetes. In Type 2 Diabetes (or “T2DM”), hyperglycemia results from a varying combination of insulin resistance and inadequate insulin production. Chronic hyperglycaemia can cause damage to various organs, leading to the development of disabling and life-threatening complications such as cardiovascular disease, neuropathy, nephropathy, and eye disease leading to retinopathy and blindness. These complications contribute to more frequent hospitalization, increased medical care costs and lower quality of life, and often results in early death. In fact, the IDF estimates that approximately four million people aged between 20 and 79 years died from diabetes in 2017, which equates to about one death every eight seconds. Diabetes accounted for 10.7% of global all-cause mortality among people in this age group, which is higher than the combined number of deaths from infectious diseases (HIV/AIDS, Tuberculosis, and Malaria). Moreover, around 46.1% of deaths due to diabetes in this age bracket were in individuals under the age of 60. In addition to the human burden of diabetes, characterized by premature mortality and low quality of life, there is also a significant economic burden imposed by the disease and its complications. The brunt of this burden is bore upon countries, healthcare systems, and more importantly, directly and direly by inflicted individuals and their families. The global projection for annual healthcare expenditure on diabetes in 2017 was USD 727 billion, corresponding to one for every eight dollars spent on healthcare. These economic costs are ever-increasing, a trend which has been best chronicled and analyzed in the United States (US). The American Diabetes Association has estimated that, after adjusting for inflation, the economic costs of diabetes increased by 26% (from USD 188 billion to USD 237.3 billion) between 2012 to 2017 in the US, which can be attributed to both an increased prevalence of diabetes, as well as an increased cost per person with diabetes. Moreover, after adjusting for both inflation and growth in diabetes prevalence, the excess medical cost per person with diabetes grew by 14% (from USD 8,417 to USD 9,601) in the same 5-year time frame.

Notwithstanding lifestyle and behavioural modification, which have been deemed largely ineffective in the treatment of T2DM, there remains a substantive treatment gap in conventional approaches (surgery and medication) to T2DM treatment that has not been adequately addressed. For surgery, this treatment gap refers to several different cohorts of people, including a large subgroup of individuals that are overweight and diabetic but not considered to be severely obese and therefore do not qualify for traditional surgical procedures. The treatment gap also refers to individuals who do qualify for these procedures but who are unreceptive or unwilling to undergo invasive, anatomy-changing and irreversible operations. Further, the treatment gap also refers to individuals who qualify for surgery, but that are unable to access treatment due to barriers associated with high cost, lack of insurance coverage, and/or available surgical skill. For medication, barriers to access that are commonly cited are high cost, marginal efficacy, and high incidences of side effects. All of these factors put together contribute to the fact that existing surgical procedures, interventions, and medication are used by less than 1% of those eligible worldwide.

Systems, devices, and methods are described herein for use in delivering and magnetically coupling magnetic implant assemblies so as to create an anastomosis in adjacent points of the digestive tract (between the duodenum and ileum, or the jejunum and the ileum). It is recognized in the present disclosure that such resulting alternate “pathway” or “short-cut” (i.e., the anastomosis) may provide an alternate pathway for nutrient-rich chyme to enter the ileum more quickly and distally, which may result in avoiding absorption in the foregut, triggering early satiety, and/or improving glucose metabolism (e.g., by mediating a supposed “incretin effect”, which is characterized by an increased secretion of Glucagon-like peptide (GLP-1), a gut hormone that stimulates insulin secretion, gene expression, and β-cell growth). In this regard, diabetes control may result from such expedited delivery of nutrient-rich chyme to the distal intestines, and may result in the emanation of a physiological signal leading to improved glucose metabolism. It is to be understood that the principles described in the present disclosure may be applied outside of the context of endoscopic anastomosis procedures, such as performing diagnostic procedures, surgical or therapeutic procedures, scientific experiments, and/or other procedures in the same and/or other environments, cavities, and/or organs not described in the present disclosure without departing from the teachings of the present disclosure.

Example embodiments will now be described below with reference to the accompanying figures, which form a part of the present disclosure.

### Example Embodiments of the Endoscopic Anastomosis System (e.g., Endoscopic Anastomosis System 100).

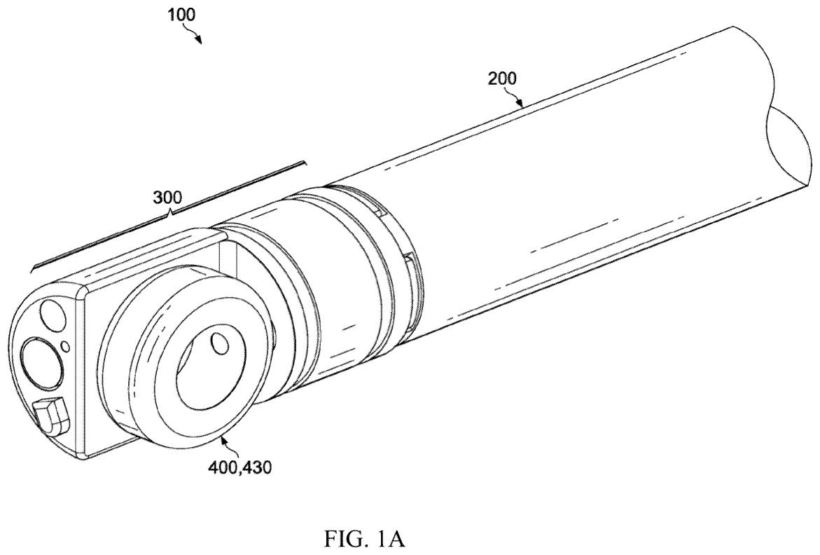

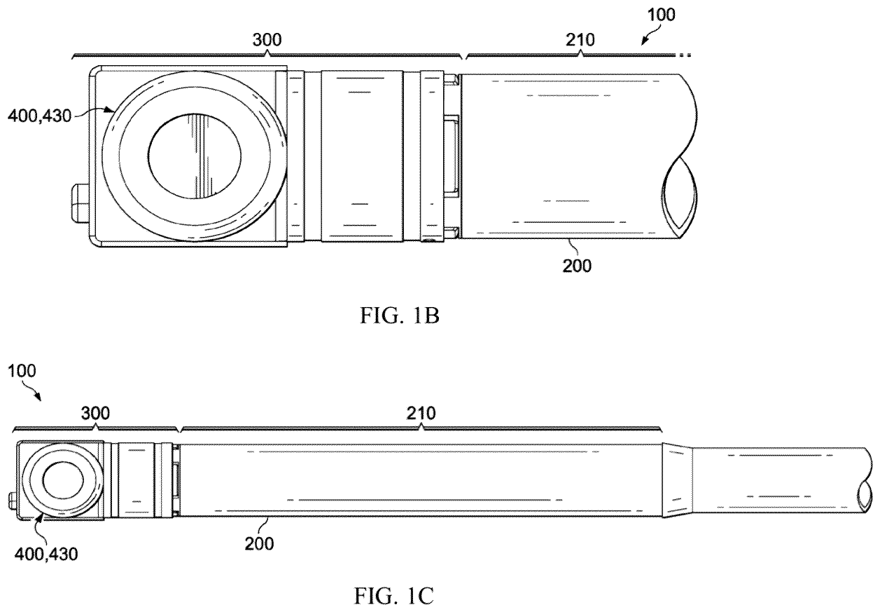

FIG. 1A, FIG. 1B, and FIG. 1C illustrate different views of an example embodiment of an endoscopic anastomosis system (e.g., system 100). The system 100 may be configurable or configured to be inserted through a natural orifice (e.g., rectum) of a patient to deliver a first magnetic implant body (or magnetic body) 430 into a cavity (e.g., colon or ileocecal valve) of the patient. A complimentary, corresponding, or associated second system 100 adapted to be inserted through another natural orifice (e.g., mouth) of the patient delivers a second magnetic implant body 430 into an adjacent cavity (e.g., duodenum or jejunum) of the patient, which is then magnetically coupled through one or more cavity walls to the first magnetic implant body 430. Together, the first and second magnetic implant bodies 430 are configured to form an anastomosis through the one or more cavity walls.

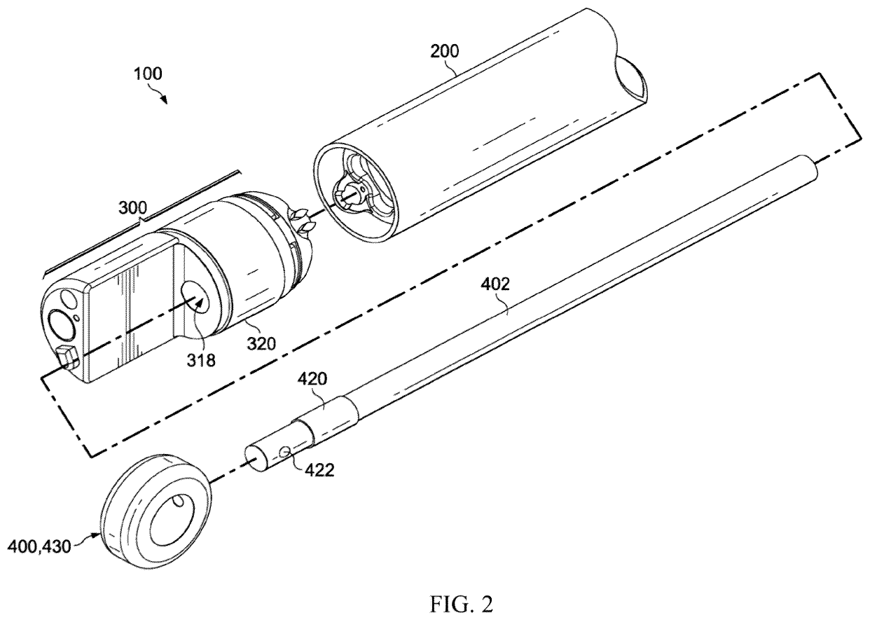

Each system 100 includes one or more elements. For example, as will be further described in the present disclosure, each system 100 includes a first main body assembly 200 (or first main body 200). Each system 100 also includes a head assembly 300. The head assembly 300 is secured to an end of the first main body assembly 200 (e.g., referred to herein as the second end 203 of the first main body assembly 200, distal end of the first main body assembly 200, or end that is inserted into a cavity of a patient). Although the head assembly 300 may be referred to in the present disclosure as being a separate element from (and secured to) the first main body assembly 200, it is to be understood that the head assembly 300 may also be considered as an element or part of the first main body assembly 200 without departing from the teachings of the present disclosure. Each system 100 also includes a second main body assembly 400. At least a portion of the second main body assembly 400 is housed in an interior of the head assembly 300, and at least a portion of the second main body assembly 400 is provided/inserted through an opening 318 (referred to herein as a “second main body assembly opening” 318, “catheter body opening” 318, or the like) of the head assembly 300. Furthermore, at least a portion of the second main body assembly 400 is housed in the first main body assembly 200. In example embodiments, the first and second main bodies 200, 400 are slidable relative to one another. Each system 100 also includes a magnetic implant assembly 430. Each system 100 also includes a securing assembly 440. Although the magnetic implant assembly 430 and/or the securing assembly 440 may be referred to in the present disclosure as being separate element(s) from (and secured to) the second main body assembly 400, it is to be understood that the magnetic implant assembly 430 and/or the securing assembly 440 may also be considered as an element or part of the second main body assembly 400 without departing from the teachings of the present disclosure. For ease of reference, FIG. 2 illustrates a view of these elements separated from one another.

As used in the present disclosure, when applicable, one or more elements of each system 100 may be controlled, in part or in whole, directly or indirectly, by or in cooperation with one or more processors, controllers, computing devices, processors, servers, systems, cloud-based computing, artificial intelligence (AI), or the like (referred to herein as a “controller”, “processor”, or the like) (not shown) and/or one or more surgeon consoles (not shown, which may be any console, or the like, for one or more surgeons to perform one or more actions described in the present disclosure). Such controller and/or surgeon console may be in communication with and/or control one or more external systems/devices (e.g., external pressure source for providing negative and/or positive pressure, etc.) (not shown). Such controller may be any processor, server, system, device, computing device, controller, microprocessor, microcontroller, microchip, semiconductor device, or the like, configurable or configured to perform, among other things, a processing and/or managing of information, searching for information, identifying of information, data communications, processing information and/or making one or more decisions via artificial intelligence, machine learning, deep learning, or the like, and/or any one or more other actions described in the present disclosure. Alternatively or in addition, such controller (and/or its elements) may include and/or be a part of a virtual machine, processor, computer, node, instance, host, or machine, including those in a networked computing environment. As used in the present disclosure, a communication channel, or the like, may be or include a collection of devices and/or virtual machines connected by communication channels that facilitate communications between devices and allow for devices to share resources. Such resources may encompass any types of resources for running instances including hardware (such as servers, clients, mainframe computers, networks, network storage, data sources, memory, central processing unit time, scientific instruments, and other computing devices), as well as software, software licenses, available network services, and other non-hardware resources, or a combination thereof. A communication channel may include, but is not limited to, the internet, intranets, WiFi systems, GPS systems, location systems, location-based service systems, computing grid systems, peer to peer systems, mesh-type systems, distributed computing environments, cloud computing environment, telephony systems, voice over IP (VOIP) systems, etc. Such communication channels may include hardware and software infrastructures configured to form a virtual organization comprised of multiple resources which may be in geographically disperse locations. Communication channel may also refer to a communication medium between processes on the same device or system.

These and other elements of the system 100 will now be described with reference to the accompanying figures.

### The First Main Body Assembly (e.g., First Main Body Assembly 200).

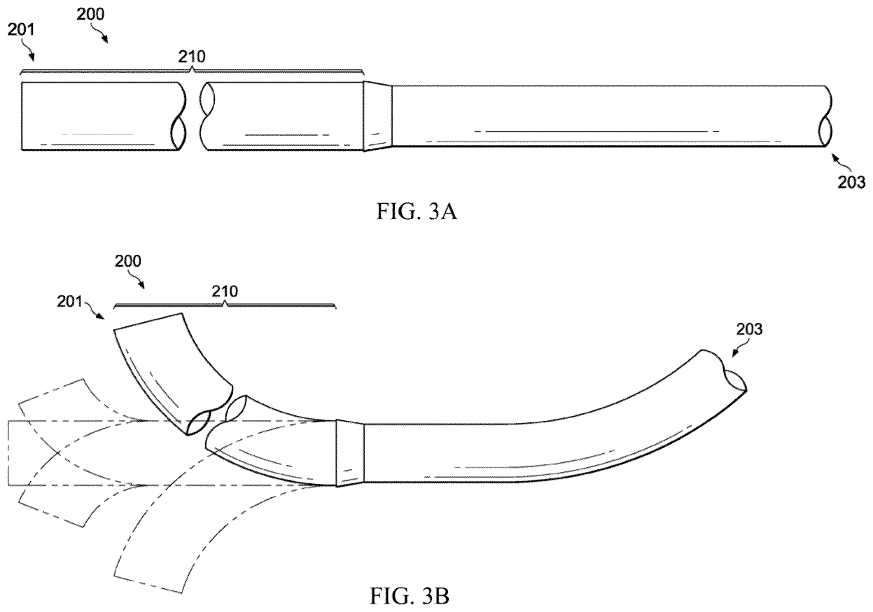

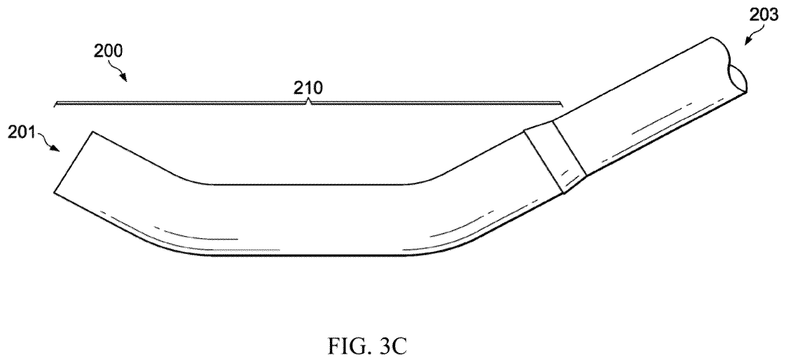

As illustrated in at least FIG. 3A and FIG. 3B, each system 200 includes a first main body assembly 200. The first main body assembly 200 may include an elongated tubular structure having a first end 201 (or “proximal end” 201) and a second end 203 (or “distal end” 203). The first main body assembly 200 may include a flexible body or tube having one or more internal channels (not shown). For example, the one or more internal channels may be provided for a plurality of actuation control members (e.g., cables, wires, tendons, or the like) to extend from the controller and/or surgeon console (at or near the first end 201) to a portion of the second end 203 (e.g., to the first bendable section 210). As another example, the one or more internal channels may be provided to house the second main body assembly 400 (which may extend from the controller and/or surgeon console (at or near the first end 201) and through the second main body assembly opening 318 of the head assembly 300. In this regard, the second main body assembly 400 and the first main body assembly 200 may be configured to slide relative to one another. As another example, the one or more internal channels may be provided to enable negative pressure and/or positive pressure to be supplied from one or more external pressure sources (not shown) to the one or more pressure openings 332, 334, 422. As another example, the one or more internal channels may be provided for positive pressure and/or negative pressure to be supplied from one or more external pressure sources (not shown) to the first and second expandable members 320, 420. As another example, the one or more internal channels may be provided for washing fluid, positive pressure, and/or negative pressure to be supplied from one or more external pressure sources (not shown) to the first cleansing assembly 338. As another example, the one or more internal channels may be provided for electrical and/or data cables to extend to the first image capturing assembly 336. As another example, the one or more internal channels may be provided for cables to extend to one or more sensors (e.g., for haptic feedback, temperature sensor, pressure sensor, etc., not shown), etc. Other internal channels for other purposes are also contemplated in the present disclosure. It is to be understood that an internal channel of the first main body assembly 200 may be any channel (including those that are wholly or partially within the first main body assembly), and may include a smaller tube, or the like, provided in a larger channel or tube. It is also to be understood that an internal channel of the first main body assembly 200 may extend beyond the first end 201 and/or second end 203 of the first main body assembly 200.

As illustrated in at least FIGS. 1A and 1B, the second end 203 of the first main body assembly 200 may be securable or secured to (and in example embodiments, detachable from) a connector portion 302 of the head assembly 300.

The first main body assembly 200 includes a first bendable section (e.g., first bendable section 210) at the second end 203 of the first main body assembly 200. Although not illustrated in the Figures, the second end 203 of the first main body assembly 200 may also include one or more expandable members (e.g., similar to the first expandable member 320 and/or second expandable member 420) and/or one or more pressure openings (e.g., similar to the first pressure opening 332 and/or second pressure opening 422). Such one or more pressure openings may be provided before and/or after such one or more expandable members, and such one or more pressure openings and/or one or more expandable members may be provided before and/or after the first bendable section 210. In some example embodiments, such expandable member(s) and/or pressure opening(s) of the first main body assembly 200 may be in addition to or replace the first expandable member 320 and/or the first pressure opening 332 of the head assembly 300.

In an example embodiment, the first bendable section 210 is configurable or configured to guide, turn, bend, and/or steer (referred to herein as “bend”, “bending”, or the like) the system 100 in any one or more of a plurality of available directions and/or one or more of a plurality of locations along the first bendable section 210. This may be desirable when the system 100 is being advanced forward into a body cavity, such as a colon or small bowel, and the system 100 reaches a bend, turn, or the like in the body cavity. Alternatively or in addition, such bending may be desirable when a particular area of the interior wall of the body cavity needs to be viewed and/or actioned (e.g., delivering of the magnetic implant body 430). Such bending of the first bendable section 210 may be achievable or achieved by selectively configuring one or more locations along the first bendable section 210 to bend (e.g., away from a center axis formed by the first bendable section 210). Such selective configuring may include selecting one or more locations along the first bendable section 210 to bend from among a plurality of bendable location(s) along the first bendable section 210. FIG. 3B illustrates an example of bending of the first bendable section 210. Selective configuring may also include selecting, for each location along the first bendable section 210, a degree of curvature for the bending from among a plurality of available degrees of curvature. Selective configuring may also include selecting, for each location along the first bendable section 210, one or more directions for the bending from among a plurality of available directions, etc.

The bending of the first bendable section 210 may be selectively controllable by controlling an amount of force (e.g., tension via pulling or pushing) applied (increased, decreased, maintained, or not applied) to one or more actuation control members (not shown) and/or selecting one or more of the actuation control members to selectively control (i.e., which actuation control member will receive an increase in applied force, decrease in applied force, no change in applied force, and/or no applied force). In an example embodiment, the first bendable section 210 may include a serially (or linearly) connected arrangement of a plurality of bendable subsections (not shown). Each bendable subsection may include one or more distal termination points for receiving, securing, terminating, and/or connecting one or more actuation control members.

Each of the bendable subsections may include one or more internal cavities or channels for, among other things, enabling one or more actuation control members to extend through, enabling negative pressure and/or positive pressure to be provided to the one or more pressure openings 332, 422, enabling positive pressure and/or negative pressure to be provided to the first expandable member 320, enabling positive pressure and/or negative pressure to be provided to the second expandable member 420, enabling fluid and/or positive pressure (and/or negative pressure) to be provided to the first cleansing assembly 338, enabling electrical and/or data cables to extend to the first image capturing assembly 336, etc.

The distal termination points may be provided in any shape or form so long as it enables the receiving, connecting, terminating, and/or securing of the distal end of one or more actuation control members. For example, the distal termination point may be an opening, connector, termination, hook, etc. A degree of bending of one or more of the bendable locations of the first bendable section 210 may be between about 0 to 210 degrees from a center axis in example embodiments.

In an example embodiment, the first main body assembly 200 may have a length between about 1600 mm to about 2200 mm, and a diameter between about 12 mm to about 18 mm. The first main body assembly 200 may be formed having one or more of a plurality of cross-sectional shapes, including a circular cross-section, elliptical cross-section, etc. Other dimensions and shapes are also contemplated without departing from the teachings of the present disclosure. A length of the first bendable section 210 may be between about 70 mm to about 130 mm, and a diameter of the first bendable section 210 may be between about 12 mm to about 18 mm in example embodiments. Other dimensions are also contemplated without departing from the teachings of the present disclosure.

### The Head Assembly (e.g., Head Assembly 300).

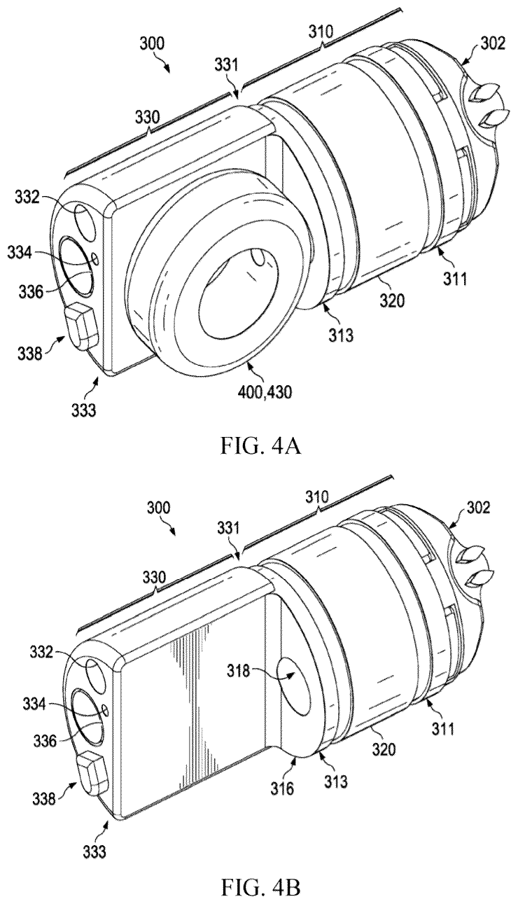

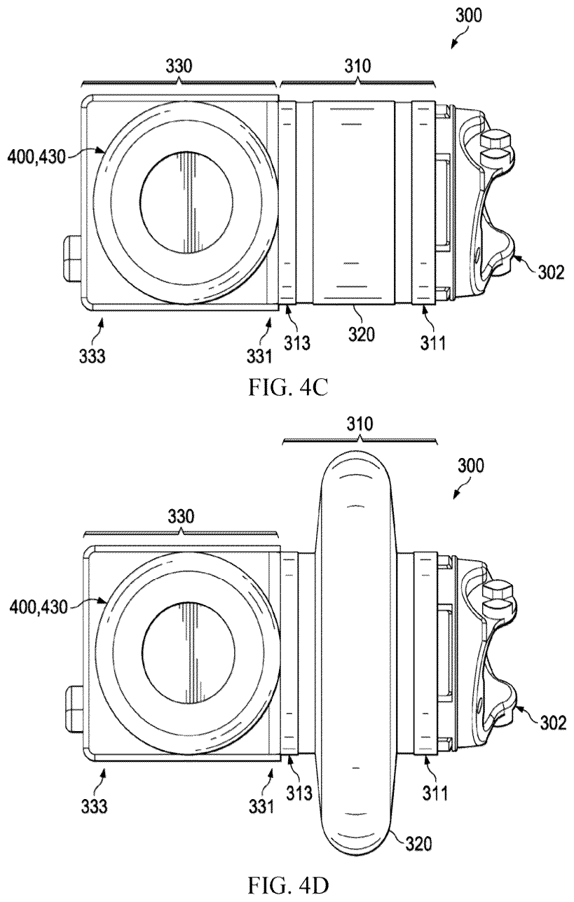

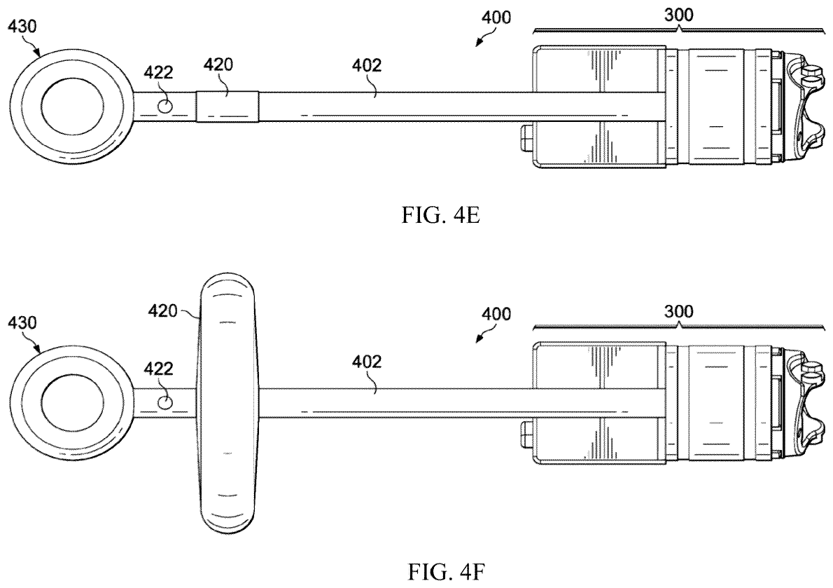

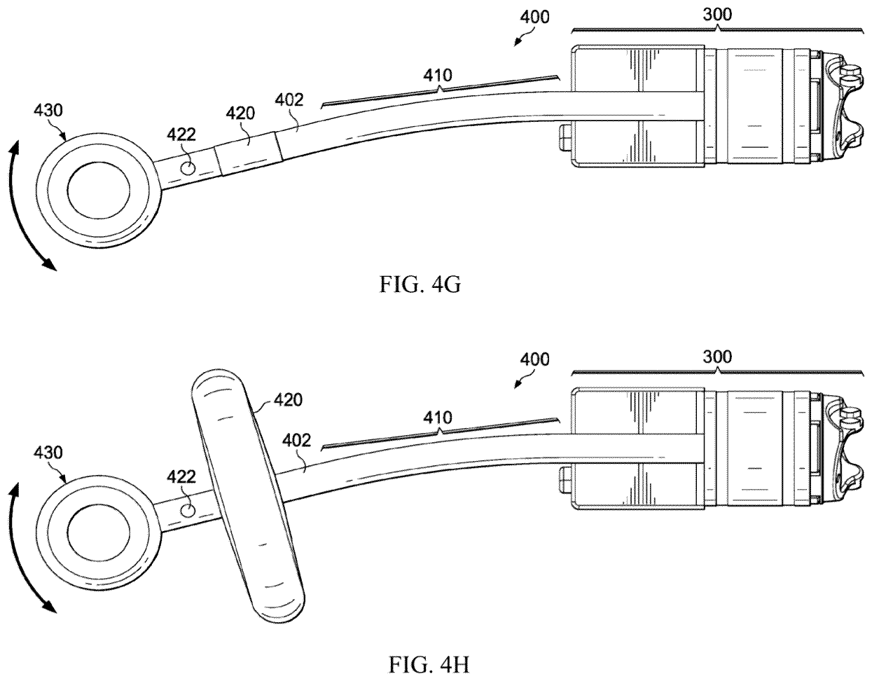

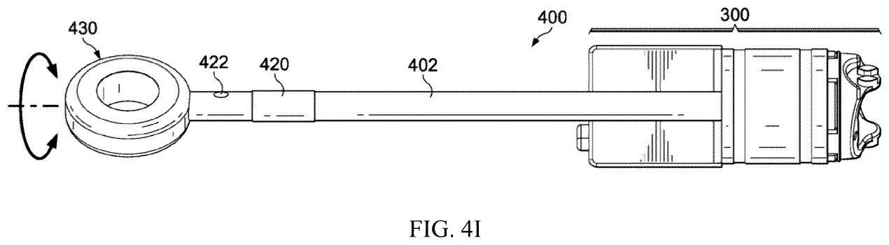

As illustrated in at least FIG. 2, FIG. 4A, and FIG. 4B, each system 200 includes a head assembly 300. The head assembly 300 includes a head assembly body 300. The head assembly 300 also includes one or more first expandable members 320. The head assembly 300 also includes one or more first pressure ports 332. The head assembly 300 also includes a second main body opening 318. The head assembly 300 also includes one or more insufflation ports 334. The head assembly 300 also includes one or more first image capturing assemblies 336. The head assembly 300 also includes one or more first cleansing assemblies 338.

These and other elements of the head assembly 300 will now be described with reference to the accompanying figures.

### The Head Assembly Body (e.g., Head Assembly Body 300).

As illustrated in at least FIGS. 2 and 4A-B, the head assembly 300 includes a head assembly body 300. The head assembly body 300 includes a connector portion 302 at a first end of the head assembly body 300 for securing to the second end 203 of the second main body assembly 200. The head assembly body 300 also includes a first region, portion, or the like (referred to herein as the “first region” 310) and a second region, portion, or the like (referred to herein as the “second region” 330). A first end 311 of the first region 310 is secured to the second end 203 of the second main body assembly 200 via the connector portion 302, and at least a portion of a second end 313 of the first region 310 is secured to the first end 331 of the second region 330.

In example embodiments, the second end 313 of the first region 310 of the head assembly body 300 includes a first section 316 (as illustrated in at least FIG. 4B) and a second section (not shown) adjacent to the first section 316. The second section of the first region 310 is secured to the first end 331 of the second region 330 of the head assembly body 300. The first section 316 of the first region 310 includes a second main body assembly opening 318 (or second main body opening 318 or catheter body opening 318 or catheter opening 318). The second main body assembly opening 318 is configured to house at least a portion of the second main body assembly 400. That is, at least a portion of the second main body assembly 400 is provided/inserted through the second main body assembly opening 318. In this regard, the second main body 402 is moveable/slidable relative to the head assembly body 300. The first region 310 also includes one or more other pressure openings (not shown) for providing positive and/or negative pressure to an interior portion of the first expandable member 320 (e.g., to expand, maintain, or contract a volume of the first expandable member 320). The first region 310 also includes one or more internal cavities or channels. For example, the one or more internal cavities or channels may house at least a portion of a second end of the second main body 402 of the second main body assembly 400. The one or more internal cavities or channels may also house the plurality of actuation control members (e.g., cables, wires, tendons, or the like, as described in the present disclosure) that control the second bendable section 410 of the second main body assembly 400. As another example, the one or more internal cavities or channels may be provided to enable negative pressure and/or positive pressure to be supplied from one or more external pressure sources (not shown) to one or more pressure openings 332, 334, 422. As another example, the one or more internal cavities or channels may be provided for positive pressure and/or negative pressure to be supplied from one or more external pressure sources (not shown) to the first and second expandable members 320, 420. As another example, the one or more internal cavities or channels may be provided for washing fluid, positive pressure, and/or negative pressure to be supplied from one or more external pressure sources (not shown) to the first cleansing assembly 338. As another example, the one or more internal cavities or channels may be provided for electrical and/or data cables to extend to the first image capturing assembly 336. As another example, the one or more internal cavities or channels may be provided for cables to extend to one or more sensors (e.g., for haptic feedback, temperature sensor, pressure sensor, etc., not shown), etc. Other internal cavities or channels for other purposes are also contemplated in the present disclosure. It is to be understood that an internal cavity or channel of the first region 310 of the head assembly body 300 may be any cavity or channel (including those that are wholly or partially within the head assembly body 300), and may include a smaller tube, or the like, provided in a larger channel or tube. It is also to be understood that an internal cavity or channel of the first region 310 of the head assembly body 300 may extend beyond the first region 310 of the head assembly body 300. The first region 310 of the head assembly body 300 may have a length between about 12 mm to about 20 mm, and a diameter between about 12 mm to about 20 mm. The first region 310 of the head assembly body 300 may be cylindrical in shape and/or formed having one or more of a plurality of cross-sectional shapes, including a circular cross-section, elliptical cross-section, etc. Other dimensions and shapes are also contemplated without departing from the teachings of the present disclosure.

In example embodiments, the first end 331 of the second region 330 of the head assembly body 300 is secured to the second section of the first region 310 of the head assembly body 300. As will be further described in the present disclosure, the second region 330 includes one or more first pressure ports 332. The second region 330 also includes one or more first insufflation ports 334. The second region 330 also includes one or more first image capturing assemblies 336. The second region 330 also includes one or more first cleansing assemblies 338. The second region 330 also includes one or more internal cavities or channels. For example, the one or more internal cavities or channels may house at least a portion of a second end of the second main body 402 of the second main body assembly 400. The one or more internal cavities or channels may also house the plurality of actuation control members (e.g., cables, wires, tendons, or the like, as described in the present disclosure) that control the second bendable section 410 of the second main body assembly 400. As another example, the one or more internal cavities or channels may be provided to enable negative pressure and/or positive pressure to be supplied from one or more external pressure sources (not shown) to one or more pressure openings 332, 334, 422. As another example, the one or more internal cavities or channels may be provided for positive pressure and/or negative pressure to be supplied from one or more external pressure sources (not shown) to the second expandable member 420. As another example, the one or more internal cavities or channels may be provided for washing fluid, positive pressure, and/or negative pressure to be supplied from one or more external pressure sources (not shown) to the first cleansing assembly 338. As another example, the one or more internal cavities or channels may be provided for electrical and/or data cables to extend to the first image capturing assembly 336. As another example, the one or more internal cavities or channels may be provided for cables to extend to one or more sensors (e.g., for haptic feedback, temperature sensor, pressure sensor, etc., not shown), etc. Other internal cavities or channels for other purposes are also contemplated in the present disclosure. It is to be understood that an internal cavity or channel of the second region 330 of the head assembly body 300 may be any cavity or channel (including those that are wholly or partially within the head assembly body 300), and may include a smaller tube, or the like, provided in a larger channel or tube. It is also to be understood that an internal cavity or channel of the second region 330 of the head assembly body 300 may extend beyond the second region 330 of the head assembly body 300. The second region 330 of the head assembly body 300 may have a length between about 14 mm to about 25 mm. The second region 330 of the head assembly body 300 may be semi-cylindrical in shape and/or formed having one or more of a plurality of cross-sectional shapes, including a semi-circular cross-section, semi-elliptical cross-section, etc. Other dimensions and shapes are also contemplated without departing from the teachings of the present disclosure.

### The First Expandable Member (e.g., First Expandable Member 320).

As illustrated in at least FIGS. 2 and 4A-D, the head assembly 300 includes one or more first expandable members (e.g., first expandable member 320). As illustrated in at least FIG. 4C, the first expandable member 320 is secured to at least a portion of the first region 310 of the head assembly 300. More specifically, the first expandable member 320 is secured to an exterior portion of the head assembly body 300 between the first and second ends 311, 313 of the first region 310 of the head assembly body 300. The first expandable member 320 is configured to transition between a normal or unexpanded configuration (e.g., as illustrated in FIG. 4C) and an expanded configuration (e.g., as illustrated in FIG. 4D) in which the first expandable member 320 expands radially outward or away from the head assembly body 300.

When the first expandable member 320 is controlled to be in the expanded configuration while in a cavity of a patient, the controller (not shown) is configured to control the first expandable member 320 to expand radially outward or away from the head assembly body 300 towards and/or to the cavity wall of the patient. When expanded, the first expandable member 320 may or may not reach the cavity wall of the patient. In situations where the first expandable member 320 (when expanded) reaches the cavity wall of the patient, the first expandable member 320 may encourage or push outward the cavity wall of the patient. However, in example embodiments, the first expandable member (when expanded) may stop just short (may not reach) or may not push outward (if it reaches) the cavity wall of the patient. In either of these situations, it is recognized that the first expandable member 320 in cooperation (or in combination) with one or more of the first pressure ports 332 (as further described in the present disclosure, which is configured to encourage, bring in, suction inward, and/or collapse a portion of the cavity wall of the patient) enables the system 100 to anchor, grip, and/or otherwise secure to the cavity wall of the patient.

The first expandable member 320 may be formed completely or partially around the first region 310 of the head assembly body 300. The first expandable member 320 may resemble a balloon, or the like, and may include one or more openings (not shown) to allow positive pressure (e.g., passage of gas and/or liquid, and/or allow a manipulation of pressure within the first expandable member 320) to be introduced, controlled, and/or reduced in the first expandable member 320. Each such opening may be connected to one or more of the pressure cavities, which are in turn connected to one or more external pressure sources (not shown). Alternatively, the first expandable member 320 may be formed via one or more membranes (e.g., rectangular sheets) of expandable material, and the opposing long sides of such membranes may be secured (e.g., via an overmolding process) to the first and second ends 311, 313 of the first region 310 of the head assembly body 300.

When in the expanded configuration, which may be a state in which the external pressure source provides a positive pressure to the first expandable member 320, the first expandable member 320 may be configurable to expand radially outward (e.g., resembling a balloon, tire, or the like) with an overall diameter of the first expandable member 320, when in the expanded configuration, between about 25 mm to 40 mm. Other dimensions are also contemplated without departing from the teachings of the present disclosure.

### The First Pressure Port (e.g., First Pressure Port 332).

As illustrated in at least FIGS. 2 and 4A-B, the head assembly 300 includes one or more first pressure ports (e.g., first pressure port 332; also referred to herein as the “first pressure opening” 332). The one or more first pressure ports 332 may be provided at the second end 333 of the second region 330, and configured to provide negative pressure (and/or positive pressure) to an exterior of the head assembly body 300 (e.g., to a cavity of a patient). For example, as illustrated in at least FIG. 4A, the one or more first pressure ports 332 may be provided on (or through) the head assembly body 300 at a most distal wall or face of the head assembly body 300 in such a way that a negative pressure applied by the one or more first pressure ports 332 is directed in a direction in which the head assembly body 300 is pointed (or advanced) (e.g., parallel to a central axis formed through the head assembly body 300). Alternatively or in addition, in example embodiments in which the one or more first pressure ports 332 are provided on (or through) the most distal wall or face of the head assembly body 300, one or more of the first pressure ports 332 may be oriented, configured, directed, or pointed in such a way that a negative pressure applied by such one or more first pressure ports 332 is directed in a direction that is not parallel to a central axis formed through the head assembly body 300 (e.g., 10-80 degrees from the central axis formed through the head assembly body 300). Alternatively or in addition, in example embodiments where more than one first pressure ports 332 are provided on (or through) the head assembly body 300, such first pressure ports 332 may be provided around a circumference of the head assembly body 300 (e.g., if a portion of the head assembly body 300 has a circular circumference, such as the first region 310; or otherwise distributed around the head assembly body 300). Alternatively or in addition, in example embodiments where more than one first pressure port 332 is provided on (or through) the head assembly body 300, such first pressure ports 330 may be provided at one or more different locations along the head assembly body 300 (e.g., in the first region 310 and/or the second region 330). The one or more first pressure openings 332 may be configured to provide a negative pressure so as to encourage, bring in, suction inward, and/or collapse a portion of cavity wall of a patient toward the head assembly body 300. It is recognized in the present disclosure that such encouraging, bringing in, suctioning inward, and/or collapsing of a portion of the cavity wall of the patient in combination with an expansion radially outward of the first expandable member 320 towards and/or to a portion of the cavity wall of the patient enables the system 100 to anchor, grip, and/or otherwise secure to the cavity wall of the patient.

Although the Figures illustrate that the first pressure port 332 is provided at the second end 333 of the second region 330 of the head assembly body 300, it is to be understood that other configurations are also contemplated in the present disclosure. For example, in addition to or in replacement of the one or more first pressure ports 332 provided at the second end 333 of the second region 330, the one or more first pressure ports 332 may be provided on a side portion (e.g., between the first and second ends 331, 333) of the second region 330 of the head assembly body 300. Alternatively or in addition, the one or more first pressure ports 332 may be provided at the second end 313 of the first region 310 of the head assembly body 300. Alternatively or in addition, the one or more first pressure ports 332 may be provided at the first end 311 of the first region 310 of the head assembly body 300.

### The First Insufflation Port (e.g., First Insufflation Port 334).

As illustrated in at least FIGS. 2 and 4A-B, the head assembly 300 includes one or more first insufflation ports (e.g., first insufflation port 334; also referred to herein as the “first insufflation opening” 334). The one or more first insufflation ports 334 may be provided at the second end 333 of the second region 330, and configured to provide positive pressure to an exterior of the head assembly body 300 (e.g., to a cavity of a patient to insufflate the cavity of the patient). For example, the one or more first insufflation openings 334 may be configured to provide a positive pressure so as to encourage, push outward, and/or expand a portion of cavity wall of a patient away from the head assembly body 300.

Although the Figures illustrate that the first insufflation port 334 is provided at the second end 333 of the second region 330 of the head assembly body 300, it is to be understood that other configurations are also contemplated in the present disclosure. For example, in addition to or in replacement of the one or more first insufflation ports 334 provided at the second end 333 of the second region 330, the one or more first insufflation ports 334 may be provided on a side portion (e.g., between the first and second ends 331, 333) of the second region 330 of the head assembly body 300. Alternatively or in addition, the one or more first insufflation ports 334 may be provided at the second end 313 of the first region 310 of the head assembly body 300. Alternatively or in addition, the one or more first insufflation ports 334 may be provided at the first end 311 of the first region 310 of the head assembly body 300.

### The First Image Capturing Assembly (e.g., First Image Capturing Assembly 336).

As illustrated in at least FIGS. 2 and 4A-B, the head assembly 300 includes one or more first image capturing assemblies (e.g., first image capturing assembly 336). The first image capturing assembly 336 may be any image and/or video capturing device including, but not limited to, a 2-D video camera and/or a 3-D stereoscopic or autostereoscopic video camera. The first image capturing assembly 336 may also include one or more illumination sources (not shown), or the like, such as one or more LED lights.

Although the Figures illustrate that the first image capturing assembly 336 is provided at the second end 333 of the second region 330 of the head assembly body 300, it is to be understood that other configurations are also contemplated in the present disclosure. For example, in addition to or in replacement of the one or more first image capturing assemblies 336 provided at the second end 333 of the second region 330, the one or more first image capturing assemblies 336 may be provided on a side portion (e.g., between the first and second ends 331, 333) of the second region 330 of the head assembly body 300. Alternatively or in addition, the one or more first image capturing assemblies 336 may be provided at the second end 313 of the first region 310 of the head assembly body 300. Alternatively or in addition, the one or more first image capturing assemblies 336 may be provided at the first end 311 of the first region 310 of the head assembly body 300.

### The First Cleansing Assembly (e.g., First Cleansing Assembly 338).

As illustrated in at least FIGS. 2 and 4A-B, the head assembly 300 includes one or more first image cleansing assemblies (e.g., first cleansing assembly 338). The first cleansing assembly 338 may be configured to direct a fluid (e.g., water, non-toxic washing fluid, etc.), positive pressure, and/or negative pressure to the first image capturing assembly 336 so as to clean, unblock, and/or otherwise improve visibility and/or image capturing quality of the first image capturing assembly 336.

Although the Figures illustrate that the first cleansing assembly 338 is provided at the second end 333 of the second region 330 of the head assembly body 300, it is to be understood that other configurations are also contemplated in the present disclosure (so long as it is nearby the first image capturing assembly 336). For example, in addition to or in replacement of the one or more first cleansing assembly 338 provided at the second end 333 of the second region 330, the one or more first cleansing assembly 338 may be provided on a side portion (e.g., between the first and second ends 331, 333) of the second region 330 of the head assembly body 300. Alternatively or in addition, the one or more first cleansing assembly 338 may be provided at the second end 313 of the first region 310 of the head assembly body 300. Alternatively or in addition, the one or more first cleansing assembly 338 may be provided at the first end 311 of the first region 310 of the head assembly body 300.

### The Second Main Body Assembly (e.g., Second Main Body Assembly 400).

As illustrated in at least FIG. 2 and FIGS. 4E-I, each system 200 includes a second main body assembly 200. The second main body assembly 400 includes one or more elements. For example, the second main body assembly 400 includes a second main body 402. The second main body assembly 400 also includes a second bendable section 410. The second main body assembly 400 also includes one or more second expandable members 420. The second main body assembly 400 also includes one or more second pressure openings 422. The second main body assembly 400 also includes a magnetic implant assembly 430. The second main body assembly 400 also includes a securing assembly 440.

These and other elements of the second main body assembly 400 will now be described with reference to the accompanying figures.

### The Second Main Body (e.g., Second Main Body 402).

As illustrated in at least FIGS. 4E-I, an example embodiment of the second main body assembly 400 includes a second main body 402. The second main body 402 may include an elongated tubular structure, or the like, having a first end (or “proximal end”) and a second end (or “distal end”, which is the end nearest and/or secured to the securing assembly 440). The second main body 402 may include a flexible body having one or more internal channels (not shown). For example, the one or more internal channels may be provided for the plurality of actuation control members (e.g., cables, wires, tendons, or the like) to extend from the controller and/or surgeon console to a portion of the second end of the second main body 402. As another example, the one or more internal channels may be provided to enable negative pressure and/or positive pressure to be supplied from one or more external pressure sources (not shown) to the one or more second pressure openings 422. As another example, the one or more internal channels may be provided for positive pressure and/or negative pressure to be supplied from one or more external pressure sources (not shown) to the second expandable member 420. As another example, the one or more internal channels may be provided for cables to control the securing assembly 440 (e.g., control the securing and unsecuring of the magnetic implant assembly 430). Other internal channels for other purposes are also contemplated in the present disclosure. It is to be understood that an internal channel of the second main body 402 may be any channel (including those that are wholly or partially within the first main body assembly), and may include a smaller tube, or the like, provided in a larger channel or tube. It is also to be understood that an internal channel of the second main body 402 may extend beyond the first end and/or second end of the second main body 402.

As illustrated in at least FIGS. 4E-I, the second end of the second main body 402 may be securable or secured to (and in example embodiments, detachable from) the securing assembly 440. The second end of the second main body 402 may also be securable or secured to (and in example embodiments, detachable from) the magnetic implant assembly 430 (e.g., via the securing assembly 440).

In an example embodiment, the second main body 400 may have a length between about 1800 mm to about 2500 mm, and a diameter between about 2 mm to about 4 mm. The second main body 400 may be formed having one or more of a plurality of cross-sectional shapes, including a circular cross-section, elliptical cross-section, etc. Other dimensions and shapes are also contemplated without departing from the teachings of the present disclosure.

### The Second Bendable Section (e.g., Second Bendable Section 410).

As illustrated in at least FIG. 4G and FIG. 4H, the second main body assembly 400 includes one or more second bendable sections (e.g., second bendable section 410). The one or more second bendable sections 410 may be provided at the second end of the second main body assembly 400.

In an example embodiment, the second bendable section 410 is configurable or configured to bend the second main body assembly 400 in any one or more of a plurality of available directions and/or one or more of a plurality of locations along the second bendable section 410. This may be desirable when the second main body assembly 400 is being advanced forward into a body cavity, such as a colon or small bowel, and the system 100 reaches a bend, turn, or the like in the body cavity. Alternatively or in addition, such bending may be desirable when a particular area of the interior wall of the body cavity needs to be viewed and/or actioned (e.g., delivering of the magnetic implant body 430). Such bending of the second bendable section 410 may be achievable or achieved by selectively configuring one or more locations along the second bendable section 410 to bend (e.g., away from a center axis formed by the second bendable section 410). Such selective configuring may include selecting one or more locations along the second bendable section 410 to bend from among a plurality of bendable location(s) along the second bendable section 410. FIGS. 4G-H illustrate an example of bending of the second bendable section 410. Selective configuring may also include selecting, for each location along the second bendable section 410, a degree of curvature for the bending from among a plurality of available degrees of curvature. Selective configuring may also include selecting, for each location along the second bendable section 410, one or more directions for the bending from among a plurality of available directions, etc.

The bending of the second bendable section 410 may be selectively controllable by controlling an amount of force (e.g., tension via pulling or pushing) applied (increased, decreased, maintained, or not applied) to one or more actuation control members (not shown) and/or selecting one or more of the actuation control members to selectively control (i.e., which actuation control member will receive an increase in applied force, decrease in applied force, no change in applied force, and/or no applied force). In an example embodiment, the second bendable section 410 may include a serially (or linearly) connected arrangement of a plurality of bendable subsections (not shown). Each bendable subsection may include one or more distal termination points for receiving, securing, terminating, and/or connecting one or more actuation control members.

Each of the bendable subsections may include one or more internal cavities or channels for, among other things, enabling one or more actuation control members to extend through, enabling negative pressure and/or positive pressure to be provided to the one or more pressure openings 422, enabling positive pressure and/or negative pressure to be provided to the second expandable member 420, enabling cables to extend to the securing assembly 440, etc.

The distal termination points for the one or more actuation control members may be provided in any shape or form so long as it enables the receiving, connecting, terminating, and/or securing of the distal end of one or more actuation control members. For example, the distal termination point may be an opening, connector, termination, hook, etc. A degree of bending of one or more of the bendable locations of the second bendable section 410 may be between about 0 to 210 degrees from a center axis in example embodiments.

In an example embodiment, a length of the second bendable section 410 may be between about 5 mm to about 50 mm, and a diameter of the second bendable section 410 may be between about 2 mm to about 4 mm in example embodiments. Other dimensions are also contemplated without departing from the teachings of the present disclosure.

Although the Figures illustrate that the sequence or order of the elements (e.g., when moving towards the securing assembly 440 or the magnetic implant assembly 430) are such that the second expandable member 420 is provided between the second bendable section 410 and the one or more second pressure ports 422, it is to be understood that other configurations are also contemplated in the present disclosure. For example, the one or more second pressure ports 422 may be provided between the second bendable section 410 and the second expandable member 420. As another example, the bendable section 410 may be provided between the second expandable member 420 and the one or more second pressure ports 422. The sequence or order of the elements may also be changed from the sequence shown in the Figures (which illustrates a sequence of the second bendable section 410, followed by the second expandable member 420, followed by the one or more second pressure ports 422). For example, the sequence may be the one or more second pressure ports 422, followed by the second expandable member 420, followed by the second bendable portion 410. As another example, the sequence may be the one or more second pressure ports 422, followed by the second bendable portion 410, followed by the second expandable member 420. As another example, the sequence may be the second expandable member 420, followed by the one or more second pressure ports 422, followed by the second bendable portion 410. As another example, the sequence may be the second expandable member 420, followed by the second bendable portion 410, followed by the one or more second pressure ports 422.

As illustrated in FIG. 4I, an example embodiment of the second main body assembly 400 is also configurable or configured to rotate relative to a central axis formed by the second main body 402. Such rotation enables an orientation of the magnetic implant assembly 430 to be selectively change, which, along with the bending of the second bendable section 410, can assist with the positioning of the magnetic implant assembly 430 for magnetically coupling to another magnetic implant assembly 430′.

### The Second Expandable Member (e.g., Second Expandable Member 420).

As illustrated in at least FIGS. 2 and FIGS. 4E-I, the second main body assembly 400 includes one or more second expandable members (e.g., second expandable member 420). As illustrated in at least FIG. 4E, the second expandable member 420 is secured to a portion of the second end of the second main body assembly 400. The second expandable member 420 is configured to transition between a normal or unexpanded configuration (e.g., as illustrated in FIG. 4E) and an expanded configuration (e.g., as illustrated in FIG. 4F and FIG. 4H) in which the second expandable member 420 is expanded radially outward or away from the second main body 402 (or towards or to a cavity wall of the patient).

When the second expandable member 420 is controlled to be in the expanded configuration while in a cavity of a patient, the controller (not shown) is configured to control the second expandable member 420 to expand radially outward or away from the second main body 402 and towards and/or to the cavity wall of the patient. When expanded, the second expandable member 420 may or may not reach the cavity wall of the patient. In situations where the second expandable member 420 (when expanded) reaches the cavity wall of the patient, the second expandable member 420 may encourage or push outward the cavity wall of the patient. However, in example embodiments, the second expandable member 420 (when expanded) may stop just short (may not reach) or may not push outward (if it reaches) the cavity wall of the patient. In either of these situations, it is recognized that the second expandable member 420 in cooperation (or in combination) with one or more of the second pressure ports 422 (as further described in the present disclosure, which is configured to encourage, bring in, suction inward, and/or collapse a portion of the cavity wall of the patient) enables the system 100 to anchor, grip, and/or otherwise secure to the cavity wall of the patient.

The second expandable member 420 may be formed completely or partially around the second main body 402. The second expandable member 420 may resemble a balloon, or the like, and may include one or more openings (not shown) to allow positive pressure (e.g., passage of gas and/or liquid, and/or allow a manipulation of pressure within the second expandable member 420) to be introduced, controlled, and/or reduced in the second expandable member 420. Each such opening may be connected to one or more of the pressure cavities, which are in turn connected to one or more external pressure sources (not shown). Alternatively, the second expandable member 420 may be formed via one or more membranes (e.g., rectangular sheets) of expandable material, and the opposing long sides of such membranes may be secured (e.g., via an overmolding process) circumferentially around the second main body 402.

When in the expanded configuration, which may be a state in which the external pressure source provides a positive pressure to the second expandable member 420, the second expandable member 420 may be configurable to expand radially outward (e.g., resembling a balloon, tire, or the like) with an overall diameter of the second expandable member 420, when in the expanded configuration, between about 10 mm to 30 mm. Other dimensions are also contemplated without departing from the teachings of the present disclosure.

Although the Figures illustrate that the sequence or order of the elements (e.g., when moving towards the securing assembly 440 or the magnetic implant assembly 430) are such that the second expandable member 420 is provided between the second bendable section 410 and the one or more second pressure ports 422, it is to be understood that other configurations are also contemplated in the present disclosure. For example, the one or more second pressure ports 422 may be provided between the second bendable section 410 and the second expandable member 420. As another example, the bendable section 410 may be provided between the second expandable member 420 and the one or more second pressure ports 422. The sequence or order of the elements may also be changed from the sequence shown in the Figures (which illustrates a sequence of the second bendable section 410, followed by the second expandable member 420, followed by the one or more second pressure ports 422). For example, the sequence may be the one or more second pressure ports 422, followed by the second expandable member 420, followed by the second bendable portion 410. As another example, the sequence may be the one or more second pressure ports 422, followed by the second bendable portion 410, followed by the second expandable member 420. As another example, the sequence may be the second expandable member 420, followed by the one or more second pressure ports 422, followed by the second bendable portion 410. As another example, the sequence may be the second expandable member 420, followed by the second bendable portion 410, followed by the one or more second pressure ports 422.

### The Second Pressure Port (e.g., Second Pressure Port 422).

As illustrated in at least FIGS. 2 and 4E-I, the second main body assembly 400 includes one or more second pressure ports (e.g., second pressure port 422; also referred to herein as the “second pressure opening” 422). The one or more second pressure ports 422 may be provided at the second end of the second main body assembly 400, and configured to provide negative pressure (and/or positive pressure) to an exterior of the second main body 402 (e.g., to a cavity of a patient). In example embodiments where more than one second pressure ports 422 are provided on (or through) the second main body 402, such second pressure ports 422 may be provided around a circumference of the second main body 402 (e.g., if the second main body 402 has a circular circumference; or otherwise around the head assembly body 300). Alternatively or in addition, in example embodiments where more than one second pressure port 422 is provided on (or through) the second main body 402, such second pressure ports 422 may be provided at one or more different locations along the second main body 402. For example, as illustrated in at least FIG. 4E, one or more second pressure ports 422 may be provided on (or through) the second main body 402 at a location between the securing assembly 440 (or most distal part of the second main body 402) and the second expandable member 420 (or most distal second expandable member 420 if there are more than one second expandable members 420). As another example (not shown), one or more second pressure ports 422 may be provided on (or through) the second main body 402 in such a way that the second expandable member 420 is provided between the one or more second pressure ports 422 and the securing assembly 440 (or most distal part of the second main body 402). As another example (not shown), one or more second pressure ports 422 may be provided on (or through) the second main body 402 before and after (or distal and proximal to) the second expandable member 420. As another example (not shown), in example embodiments in which there are two or more second expandable members 420 secured to the second main body 402, a plurality of second pressure ports 422 may be provided on (or through) the second main body 402 in such a way that second pressure ports 422 are provided before and after (or distal and proximal to) each of the second expandable members 420. The one or more second pressure ports 422 may be configured to provide a negative pressure so as to encourage, bring in, suction inward, and/or collapse a portion of cavity wall of a patient toward the second main body 402. It is recognized in the present disclosure that such encouraging, bringing in, suctioning inward, and/or collapsing of a portion of the cavity wall of the patient in combination with an expansion radially outward of the one or more second expandable members 420 towards and/or to a portion of the cavity wall of the patient enables the second main body assembly 400 to anchor, grip, and/or otherwise secure to the cavity wall of the patient.

Although the Figures illustrate that the sequence or order of the elements (e.g., when moving towards the securing assembly 440 or the magnetic implant assembly 430) are such that the second expandable member 420 is provided between the second bendable section 410 and the one or more second pressure ports 422, it is to be understood that other configurations are also contemplated in the present disclosure. For example, alternatively or in addition, the one or more second pressure ports 422 may be provided between the second bendable section 410 and the second expandable member 420. As another example, alternatively or in addition, the bendable section 410 may be provided between the second expandable member 420 and the one or more second pressure ports 422. The sequence or order of the elements may also be changed from the sequence shown in the Figures (which illustrates a sequence of the second bendable section 410, followed by the second expandable member 420, followed by the one or more second pressure ports 422). For example, the sequence may be the one or more second pressure ports 422, followed by the second expandable member 420, followed by the second bendable portion 410. As another example, the sequence may be the one or more second pressure ports 422, followed by the second bendable portion 410, followed by the second expandable member 420. As another example, the sequence may be the second expandable member 420, followed by the one or more second pressure ports 422, followed by the second bendable portion 410. As another example, the sequence may be the second expandable member 420, followed by the second bendable portion 410, followed by the one or more second pressure ports 422.

### The Magnetic Implant Assembly (e.g., Magnetic Implant Assembly 430).

As illustrated in at least FIGS. 2 and 4E-I, the second main body assembly 400 includes a magnetic implant assembly (e.g., magnetic implant assembly 430, magnetic implant body 430, magnetic body 430, or the like). The magnetic implant assembly 430 is securable to and unsecurable from the second main body 402 via the securing assembly 440. As will be further described in the present disclosure, the magnetic implant assembly 430 may be formed, in whole or in part, as or using ferromagnetic or magnetic materials.

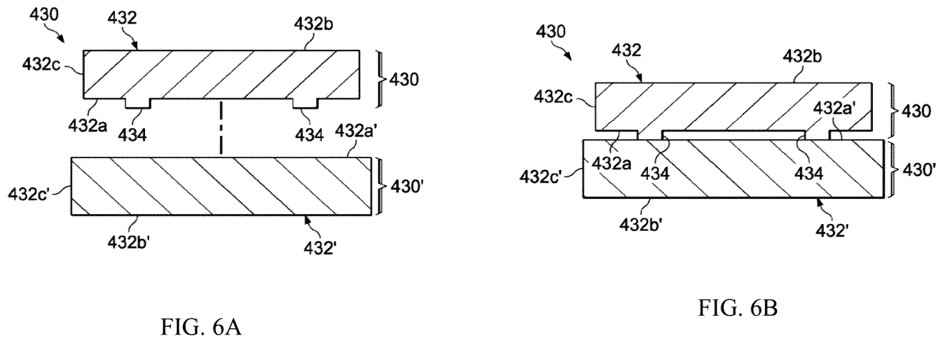

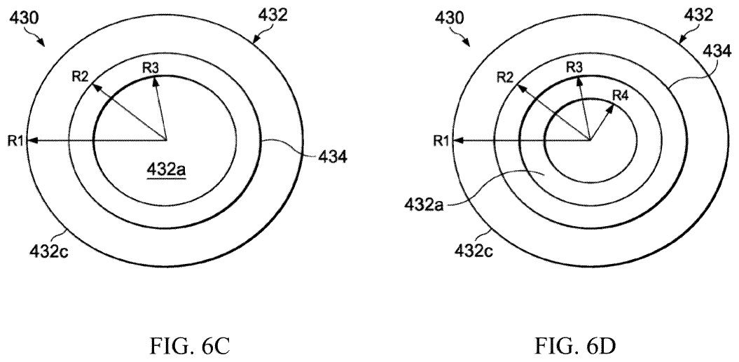

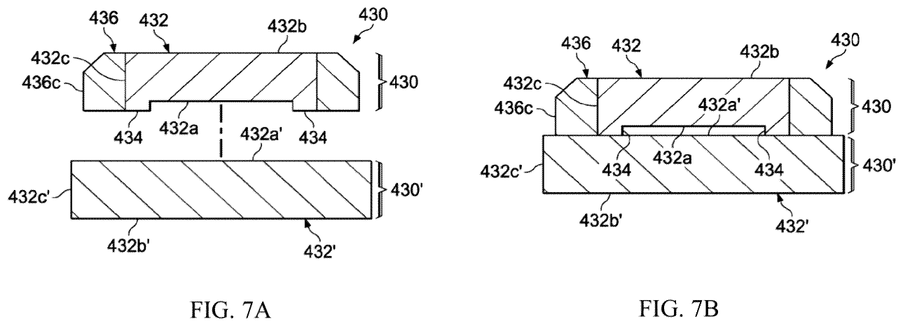

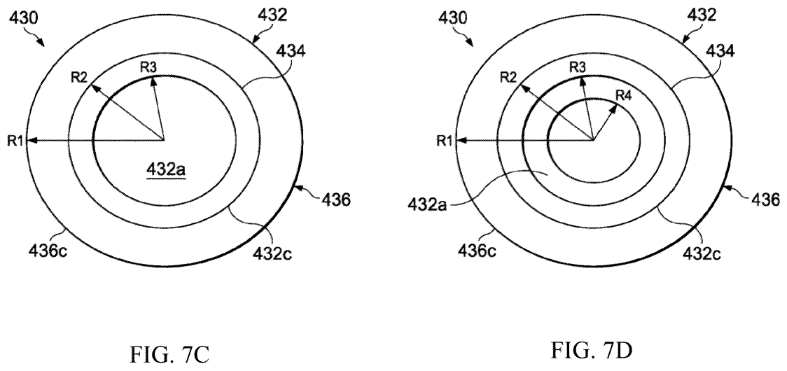

The magnetic implant assembly 430 may be configured in one or more configurations. In this regard, an example embodiment of a first magnetic implant assembly 430 of one system 100 (e.g., a first system 100 for delivering of the magnetic implant assembly 430 orally via entry through a mouth of the patient) may or may not be the same as an example embodiment of a second magnetic implant assembly 430 of another system 100 (e.g., a second system 100 for delivering of the magnetic implant assembly 430 rectally via entry through the rectum of the patient). For example, as illustrated in FIGS. 6A, 6B, 7A, and 7B and as will be further described in the present disclosure, the second magnetic implant assembly 430′ may not include any protrusions 434 and/or indentations 435 and the first magnetic implant assembly 430 may include one or more protrusions 434 and/or one or more indentations 435.

Example embodiments of the magnetic implant assembly 430 will now be described with reference to FIGS. 6A-D and 7A-D.

### First Example Embodiment of the Magnetic Implant Assembly 430.

As illustrated in the cross-sectional side views of FIG. 6A and FIG. 6B and the top views of FIG. 6C and FIG. 6D, a first magnetic implant assembly 430 may be formed as and/or include a flat and cylindrical body 432 (e.g., flat when viewed from the side and circular when viewed from the top). The body 432 is formed, in whole or in part, as or using a ferromagnetic or magnetic material.

The first magnetic implant assembly 430 includes a front wall 432a (e.g., the lower wall 432a of the upper magnetic implant assembly 430 illustrated in FIGS. 6A and 6B; the wall 432a illustrated in FIGS. 6C and 6D). In example embodiments, the front wall 432a is the wall that will be magnetically coupled to or facing the second magnetic implant assembly 430′. The front wall 432a may have a circular shape with a central axis formed through the center of the front wall 432a. The front wall 432a may have a radius of R1 (from the central axis), as illustrated in FIGS. 6C-D. In an example embodiment, the first magnetic implant assembly 430 may also include a hole, bore, or the like, through the center axis, as illustrated in at least FIG. 6D. In such embodiments, the hole may have a radius of R4 (from the central axis).

The first magnetic implant assembly 430 includes a rear wall 432b opposite to the front wall 432a. In example embodiments, the rear wall 432b is not the wall that will be magnetically coupled to or facing the second magnetic implant assembly 430′. The rear wall 432b will have substantially the same shape and central axis as the front wall 432a, and radius R1 (from the central axis).

The first magnetic implant assembly 430 includes a first exterior circumferential sidewall 432c formed around the magnetic body 432. The first exterior circumferential sidewall 432c may define a thickness of the magnetic body 432. The first exterior circumferential sidewall 432c may be formed at the radius R1 from the central axis.

In an example embodiment, the first magnetic implant assembly 430 includes one or more protrusions 434 formed on the front wall 432a. The one or more protrusions 434 may be formed in one or more of a plurality of shapes or forms. The one or more protrusions 434 may be formed using ferromagnetic or magnetic material. For example, as illustrated in FIGS. 6A-D, the protrusion 434 may be in the shape of a protruded ring having an exterior radius of R2 and an interior radius of R3. Alternatively or in addition, the first magnetic implant assembly 430 may include another protrusion (not shown) formed on the protrusion that has radiuses R2 and R1 (e.g., in a stepped manner), where the additional protrusion has an exterior radius between R2 and R1. One or more other protrusions may also be formed on the front wall 432a and/or another protrusion 432 without departing from the teachings of the present disclosure. In example embodiments, one or more of the protrusions 434 may not necessarily be formed using the same ferromagnetic or magnetic material (and/or the same magnetic force) as the body 432. For example, one or more of the protrusions 434 may be formed as or using a weaker magnet (or ferromagnetic or magnetic material having a weaker magnetic force) as compared to the ferromagnetic or magnetic material of the body 432. As another example, one or more of the protrusions 434 may be formed as or using a stronger magnet (or ferromagnetic or magnetic material having a stronger magnetic force) as compared to the ferromagnetic or magnetic material of the first magnetic implant assembly 430. As another example, one or more of the protrusions 434 may not be formed using (and/or may not include) a ferromagnetic or magnetic material, and instead may be formed using other materials such as plastic, silicon rubber, etc. Other configurations, magnetic coupling strengths, and/or materials/compositions are also contemplated without departing from the teachings of the present disclosure.

In example embodiments where the first magnetic implant assembly 430 includes a circular or ring-shaped protrusion 434 formed on the front wall 432a, the second magnetic implant assembly 430′ may be formed as and/or include a flat and cylindrical body 432′ (e.g., flat when viewed from the side and circular when viewed from the top).

The second magnetic implant assembly 430′ includes a front wall 432a′ (e.g., the upper wall 432a′ of the lower magnetic implant assembly 430′ illustrated in FIGS. 6A and 6B). In example embodiments, the front wall 432a′ is the wall that will be magnetically coupled to or facing the second magnetic implant assembly 430′. The front wall 432a′ may have a circular shape with a central axis formed through the center of the front wall 432a′. The front wall 432a′ may have a radius equal or not equal to R1 (from the central axis), but larger than radius R2. In an example embodiment, the second magnetic implant assembly 430′ may also include a hole, bore, or the like, through the center axis, similar to that of the first magnetic implant assembly 430.

The second magnetic implant assembly 430′ includes a rear wall 432b′ opposite to the front wall 432a′. In example embodiments, the rear wall 432b′ is not the wall that will be magnetically coupled to or facing the first magnetic implant assembly 430. The rear wall 432b′ will have substantially the same shape and central axis as the front wall 432a′, and radius (from the central axis).

The second magnetic implant assembly 430′ includes a second exterior circumferential sidewall 432c′ formed around the magnetic body 432′. The second exterior circumferential sidewall 432c′ may define a thickness of the magnetic body 432′. The thickness of the magnetic body 432′ may or may not be the same as the thickness of the magnetic body 432 of the first magnetic implant assembly 430.

In an example embodiment, the front wall 432a′ of the second magnetic implant assembly 430′ does not include any protrusions like that of the first magnetic implant assembly 430. It is recognized in the present disclosure that not having protrusions on the front wall 432a′ of the second magnetic implant assembly 430′ allows for a simple and aligned magnetic coupling with the front wall 432a (i.e., with the protrusions 434) of the first magnetic implant assembly 430 (as illustrated in FIG. 6B). It is to be understood, however, that the front wall 432a′ of the second magnetic implant assembly 430′ may also include one or more protrusions (not shown) similar to (e.g., different exterior and/or interior radiuses) or the same as (e.g., same exterior and/or interior radiuses) the protrusions 434 of the front wall 432 of the first magnetic implant assembly 430.

It is recognized in the present disclosure that having an exterior portion of the first magnetic implant assembly 430 (i.e., the portion between radius R1 and radius R2) not being magnetically coupled to the second magnetic implant assembly 430′ (or being magnetically coupled to the second magnetic implant assembly 430′ with a lesser magnetic force due to the airgap between the exterior portion of the first magnetic implant assembly 430 and the second magnetic implant assembly 430′) results in a force or pressure (F1) exerted on a first portion of the cavity wall of the patient (i.e., force between the exterior portion of the first magnetic implant assembly 430 between R1 and R2 and the second magnetic implant assembly 430′) to be less than a force or pressure (F2) exerted on a second (adjacent) portion of the cavity wall of the patient (i.e., force between the protrusion 434 and the second magnetic implant assembly 430′). In this regard, it is recognized in the present disclosure that the adjacent application of different forces or pressures, as described above and in the present disclosure, improves the healing (and/or enables better controlled healing) of the anastomosis and/or necrosis formed by the first and second magnetic implant assemblies 430, 430′.

It is to be noted in the present disclosure that the first magnetic implant assembly 430 (and/or the second magnetic implant assembly 430′) may also include one or more indentations on the front wall 432a (and 432a′). For example, the section of the front wall 432a between the exterior radius R2 and the radius R1 may be an indentation. As another example, the section of the front wall 432a between the interior radius R3 and the central axis (or the radius R4 for the example embodiment illustrated in FIG. 6D) may be an indentation.

### Second Example Embodiment of the Magnetic Implant Assembly 430.

As illustrated in the cross-sectional side views of FIG. 7A and FIG. 7B and the top views of FIG. 7C and FIG. 7D, a first magnetic implant assembly 430 may be formed as and/or include a flat and cylindrical body 432 (e.g., flat when viewed from the side and circular when viewed from the top). The body 432 is formed, in whole or in part, as or using a ferromagnetic or magnetic material.

The first magnetic implant assembly 430 includes a front wall 432a (e.g., the lower wall 432a of the upper magnetic implant assembly 430 illustrated in FIGS. 7A and 7B; the wall 432a illustrated in FIGS. 7C and 7D). In example embodiments, the front wall 432a is the wall that will be magnetically coupled to or facing the second magnetic implant assembly 430′. The front wall 432a may have a circular shape with a central axis formed through the center of the front wall 432a. The front wall 432a may have a radius of R2 (from the central axis), as illustrated in FIGS. 7C-D. In an example embodiment, the first magnetic implant assembly 430 may also include a hole, bore, or the like, through the center axis, as illustrated in at least FIG. 7D. In such embodiments, the hole may have a radius of R4 (from the central axis).

The first magnetic implant assembly 430 includes a rear wall 432b opposite to the front wall 432a. In example embodiments, the rear wall 432b is not the wall that will be magnetically coupled to or facing the second magnetic implant assembly 430′. The rear wall 432b will have substantially the same shape and central axis as the front wall 432a, and radius R1 (from the central axis).

The first magnetic implant assembly 430 includes a first exterior circumferential sidewall 432c formed around the magnetic body 432. The first exterior circumferential sidewall 432c may define a thickness of the magnetic body 432 (and protrusion 434, as further described below). The first exterior circumferential sidewall 432c may be formed at the radius R2 from the central axis.

In an example embodiment, the first magnetic implant assembly 430 includes one or more protrusions 434 formed on the front wall 432a. The one or more protrusions 434 may be formed in one or more of a plurality of shapes or forms. The one or more protrusions 434 may be formed using ferromagnetic or magnetic material. For example, as illustrated in FIGS. 7A-D, the protrusion 434 may be in the shape of a protruded ring having an exterior radius of R2 and an interior radius of R3. One or more other protrusions may also be formed on the front wall 432a without departing from the teachings of the present disclosure. In example embodiments, one or more of the protrusions 434 may not necessarily be formed using the same ferromagnetic or magnetic material (and/or the same magnetic force) as the body 432. For example, one or more of the protrusions 434 may be formed as or using a weaker magnet (or ferromagnetic or magnetic material having a weaker magnetic force) as compared to the ferromagnetic or magnetic material of the body 432. As another example, one or more of the protrusions 434 may be formed as or using a stronger magnet (or ferromagnetic or magnetic material having a stronger magnetic force) as compared to the ferromagnetic or magnetic material of the first magnetic implant assembly 430. As another example, one or more of the protrusions 434 may not be formed using (and/or may not include) a ferromagnetic or magnetic material, and instead may be formed using other materials such as plastic, silicon rubber, etc. Other configurations, magnetic coupling strengths, and/or materials/compositions are also contemplated without departing from the teachings of the present disclosure.

As illustrated in FIGS. 7A-B, the first magnetic implant assembly 430 includes an exterior cylindrical or ring-shaped body 436. The exterior body 436 is formed around and fixedly secured to the first exterior circumferential sidewall 432c of the magnetic body 432. The exterior body 436 includes a front exterior cylindrical or ring-shaped portion, at least a portion of which is adjacent to the front wall 432a. The exterior body 436 may also include a rear exterior cylindrical or ring-shaped portion adjacent to the rear wall 432b. In an example embodiment, the exterior body 436 may or may not be a wholly or fully magnetic body and/or may or may not be magnetically coupled to the second magnetic implant assembly 430′ when the first magnetic implant assembly 430 is magnetically coupled to the second magnetic implant assembly 430′. For example, the exterior body 436 may not be formed using (and/or may not include) a ferromagnetic or magnetic material, and instead may be formed using other materials such as plastic, silicon rubber, etc. As another example, the exterior body 436 may be formed as or using a weaker magnet (or ferromagnetic or magnetic material having a weaker magnetic force) as compared to the ferromagnetic or magnetic material of the body 432 and/or the protruding portion 434. As another example, the exterior body 436 may be partially formed as or using one or more magnets and/or magnetic sections (or ferromagnetic or magnetic material) having a same, weaker, and/or stronger magnetic force as compared to the ferromagnetic or magnetic material of the body 432 and/or the protruding portion 434. It is recognized in the present disclosure that having the exterior body 436 not being magnetically coupled to the second magnetic implant assembly 430′ (or being magnetically coupled to the second magnetic implant assembly 430′ with a lesser magnetic force) results in a force or pressure (F1) exerted on a first portion of the cavity wall of the patient (i.e., force between the exterior body 436 and the second magnetic implant assembly 430′) to be less than a force or pressure (F2) exerted on a second (adjacent) portion of the cavity wall of the patient (i.e., force between the protrusion 434 and the second magnetic implant assembly 430′). In this regard, it is recognized in the present disclosure that the adjacent application of different forces or pressures, as described above and in the present disclosure, improves the healing (and/or enables better controlled healing) of the anastomosis and/or necrosis formed by the first and second magnetic implant assemblies 430, 430′.

In example embodiments where the first magnetic implant assembly 430 includes a circular or ring-shaped protrusion 434 formed on the front wall 432a, the second magnetic implant assembly 430′ may be formed as and/or include a flat and cylindrical body 432′ (e.g., flat when viewed from the side and circular when viewed from the top).

The second magnetic implant assembly 430′ includes a front wall 432a′ (e.g., the upper wall 432a′ of the lower magnetic implant assembly 430′ illustrated in FIGS. 7A and 7B). In example embodiments, the front wall 432a′ is the wall that will be magnetically coupled to or facing the second magnetic implant assembly 430′. The front wall 432a′ may have a circular shape with a central axis formed through the center of the front wall 432a′. The front wall 432a′ may have a radius equal or not equal to R1 (from the central axis), but larger than radius R2. In an example embodiment, the second magnetic implant assembly 430′ may also include a hole, bore, or the like, through the center axis, similar to that of the first magnetic implant assembly 430.

The second magnetic implant assembly 430′ includes a rear wall 432b′ opposite to the front wall 432a′. In example embodiments, the rear wall 432b′ is not the wall that will be magnetically coupled to or facing the first magnetic implant assembly 430. The rear wall 432b′ will have substantially the same shape and central axis as the front wall 432a′, and radius (from the central axis).

The second magnetic implant assembly 430′ includes a second exterior circumferential sidewall 432c′ formed around the magnetic body 432′. The second exterior circumferential sidewall 432c′ may define a thickness of the magnetic body 432′ and the protrusion 434. The thickness of the magnetic body 432′ may or may not be the same as the thickness of the magnetic body 432 of the first magnetic implant assembly 430.

In an example embodiment, the front wall 432a′ of the second magnetic implant assembly 430′ does not include any protrusions like that of the first magnetic implant assembly 430. It is recognized in the present disclosure that not having protrusions on the front wall 432a′ of the second magnetic implant assembly 430′ allows for a simple and aligned magnetic coupling with the front wall 432a (i.e., with the protrusions 434) of the first magnetic implant assembly 430 (as illustrated in FIG. 7B). It is to be understood, however, that the front wall 432a′ of the second magnetic implant assembly 430′ may also include one or more protrusions (not shown) similar to (e.g., different exterior and/or interior radiuses) or the same as (e.g., same exterior and/or interior radiuses) the protrusions 434 of the front wall 432 of the first magnetic implant assembly 430.

It is to be noted in the present disclosure that the first magnetic implant assembly 430 (and/or the second magnetic implant assembly 430′) may also include one or more indentations 435 on the front wall 432a (and 432a′). For example, the section 435 of the front wall 432a between the interior radius R3 and the central axis (or the radius R4 for the example embodiment illustrated in FIG. 6D) may be an indentation.

It is to be understood in the present disclosure that an exterior body (e.g., similar to the exterior body 436 described above for the first magnetic implant assembly 430) may be formed around and fixedly secured to the second exterior circumferential sidewall 432c′ of the second magnetic implant assembly 430′ in addition to or in replacement of the exterior body 436 for the first magnetic implant assembly 430 without departing from the teachings of the present disclosure.

### The Securing Assembly (e.g., Securing Assembly 440).

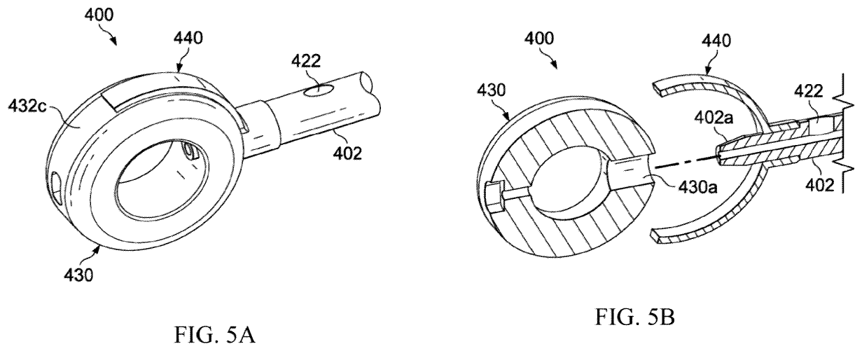

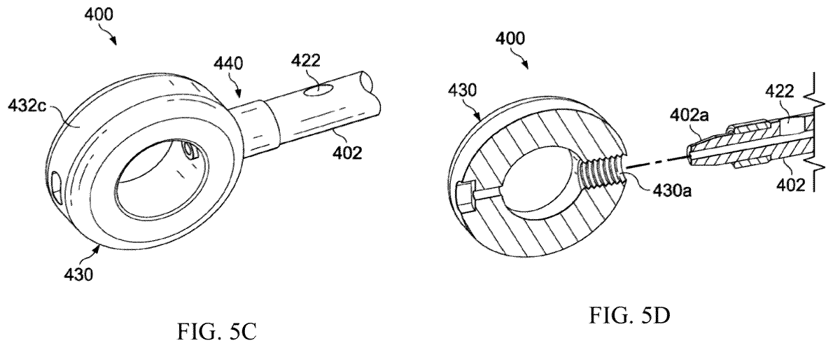

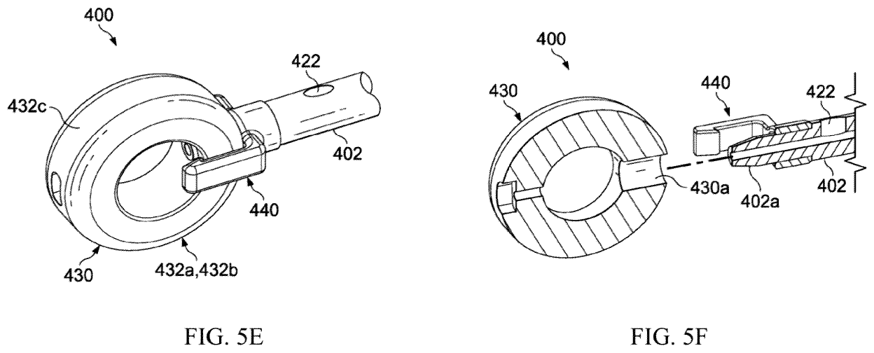

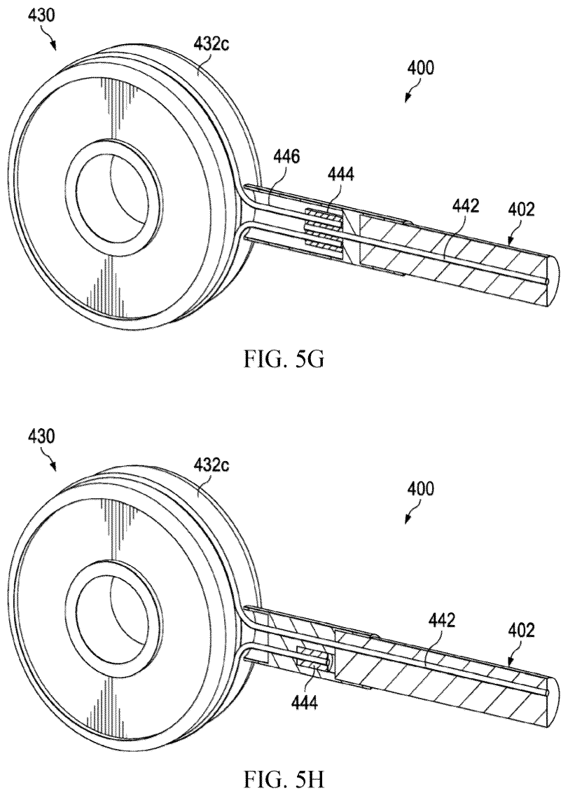

As illustrated in at least FIGS. 2 and 5A-H, the second main body assembly 400 includes a securing assembly (e.g., securing assembly 440). The securing assembly 440 is configured to secure the magnetic implant assembly 430 to and unsecure the magnetic implant assembly 430 from the second main body 402.

The securing assembly 440 may be configured in one or more configurations. For example, as illustrated in FIGS. 5A-B and FIGS. 5E-F and as will be further described in the present disclosure, the securing assembly 440 may be configured in the form of a gripper 440, or the like. As another example, as illustrated in FIGS. 5C-D and as will be further described in the present disclosure, the securing assembly 440 may be configured in the form of a quarter or half rotation screw lock 440. As another example, as illustrated in FIGS. 5G-H and as will be further described in the present disclosure, the securing assembly 440 may be configured using a snare 440, or the like.

Example embodiments of the securing assembly 440 will now be described with reference to FIGS. 5A-H.

### First Example Embodiment of the Securing Assembly 440.

As illustrated in the perspective view of FIG. 5A and cross-sectional view of FIG. 5B, a securing assembly 440 may be formed as and/or include a gripper 440, or the like, for gripping at least a portion of the exterior circumferential sidewall 432c of the magnetic implant assembly 430. In an example embodiment, the magnetic implant assembly 430 may include a hole 430a for use in receiving a protruding portion 402a of the second main body 402. In example embodiments, the gripper 440 may be sized so as to grip more than half of the circumference or perimeter of the magnetic implant assembly 430.

In example embodiments, a wire or cable (not shown) may be secured at one end to a portion of the magnetic implant assembly 430 and provided through the hole 430a, second main body 402, and back to the controller (not shown) and/or surgeon console. Such a wire or cable may be useful in situations where the magnetic implant assembly 430 is accidentally, unintentionally, or mistakenly unsecured from the securing assembly 440. Such a wire or cable may be cut or disconnected at or close to the magnetic implant assembly 430 upon the magnetic implant assembly 430 being magnetically coupled to another magnetic implant assembly 430′.

### Second Example Embodiment of the Securing Assembly 440.

As illustrated in the perspective view of FIG. 5C and cross-sectional view of FIG. 5D, a securing assembly 440 may be formed as and/or include a screw lock 440, or the like, for securing and unsecuring the magnetic implant assembly 430. More specifically, the magnetic implant assembly 430 may include a quarter or half rotation (or other) threaded hole 430a for use in receiving a screw-like protruding portion 402a of the second main body 402.

As described above and in the present disclosure, a wire or cable (not shown) may be secured at one end to a portion of the magnetic implant assembly 430 and provided through the hole 430a, second main body 402, and back to the controller (not shown) and/or surgeon console (not shown).

### Third Example Embodiment of the Securing Assembly 440.

As illustrated in the perspective view of FIG. 5E and cross-sectional view of FIG. 5F, a securing assembly 440 may be formed as and/or include a gripper 440, or the like, for gripping at least a portion of the front wall 432a and rear wall 432b (and/or at least a portion of a protrusion (not shown) similar to the protrusions illustrated in FIGS. 6A-D and 7A-D) of the magnetic implant assembly 430. In an example embodiment, the magnetic implant assembly 430 may include a hole 430a for use in receiving a protruding portion 402a of the second main body 402.

As described above and in the present disclosure, a wire or cable (not shown) may be secured at one end to a portion of the magnetic implant assembly 430 and provided through the hole 430a, second main body 402, and back to the controller (not shown) and/or surgeon console.

### Fourth and Fifth Example Embodiments of the Securing Assembly.

As illustrated in the perspective view of FIG. 5G, the securing assembly may be formed as and/or include a snare, or the like, for securing and unsecuring the magnetic implant assembly 430. More specifically, the magnetic implant assembly 430 includes a first cable 442 secured at one end to an actuatable member 444 and provided through the system 100 to a second end (i.e., at the end where the controller (not shown) or surgeon console (not shown) is located). The magnetic implant assembly 430 also includes a second cable 446 secured at both ends to the actuatable member 444. The second cable 446 is fittedly provided in a groove, channel, or the like, formed around the exterior circumferential sidewall 432c of the magnetic implant assembly 430 so as to prevent the second cable 446 from sliding around relative to and/or coming away from the exterior circumferential sidewall 432c.

When the magnetic implant assembly 430 is to be secured to the second main body 402, the first cable 442 is persistently pulled at the second end in such a way that the actuatable member 444 is persistently held in a first (secured) position (i.e., a furthest position from the magnetic implant assembly 430 (or a furthest position from the most distal point of the second end of the second main body 402)). At the first (secured) position, the second cable 446 is persistently held in the groove of the exterior circumferential sidewall 432c, and the magnetic implant assembly 430 is accordingly secured to the second main body 402.

When the magnetic implant assembly 430 is to be unsecured from the second main body 402 (e.g., when the magnetic implant assembly 430 is to be magnetically coupled to another magnetic implant assembly 430′), the first cable 442 is released or pushed from the second end in such a way that the actuatable member 444 is moved to a second (unsecured) position (i.e., a position that is closer to the magnetic implant assembly 430 (or a position that is closer to the most distal point of the second end of the second main body 402)). At the second (unsecured) position, the second cable 446 is no longer persistently held in the groove of the exterior circumferential sidewall 432c (i.e., becomes looser, becomes a bigger loop), and the magnetic implant assembly 430 is accordingly unsecured or unsecurable from the second main body 402.

The securing assembly may be formed as and/or include other configurations of a snare, or the like, for securing and unsecuring the magnetic implant assembly 430. For example, as illustrated in FIG. 5H, the magnetic implant assembly 430 includes a first cable 442 secured at one end to an actuatable member 444, provided in a groove, channel, or the like, formed around the exterior circumferential sidewall 432c of the magnetic implant assembly 430, provided through the second main body 402, and provided through the rest of the system 100 to a second end (i.e., at the end where the controller (not shown) or surgeon console (not shown) is located). The first cable 442 is fittedly provided in the groove formed around the exterior circumferential sidewall 432c of the magnetic implant assembly 430 so as to prevent the second cable 446 from sliding around relative to and/or coming away from the exterior circumferential sidewall 432c.

When the magnetic implant assembly 430 is to be secured to the second main body 402, the first cable 442 is persistently pulled at the second end in such a way that the magnetic implant assembly 430 is persistently secured by the first cable 442.

When the magnetic implant assembly 430 is to be unsecured from the second main body 402 (e.g., when the magnetic implant assembly 430 is to be magnetically coupled to another magnetic implant assembly 430′), the first cable 442 is released or pushed from the second end in such a way that the first cable 442 is no longer persistently held in the groove of the exterior circumferential sidewall 432c (i.e., becomes looser, becomes a bigger loop), and the magnetic implant assembly 430 is accordingly unsecured or unsecurable from the second main body 402.

Other embodiments and/or configurations of snares are also contemplated in the present disclosure.

As described above and in the present disclosure, a wire or cable (not shown) may be secured at one end to a portion of the magnetic implant assembly 430 and provided through the hole 430a, second main body 402, and back to the controller (not shown) and/or surgeon console.

### Example Embodiments of a Method of Delivering a Magnetic Implant Assembly Rectally.

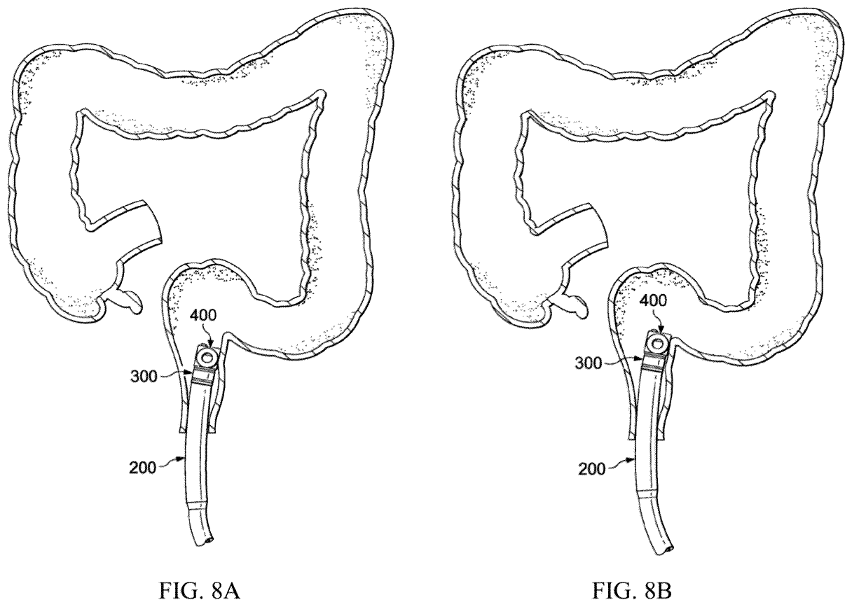

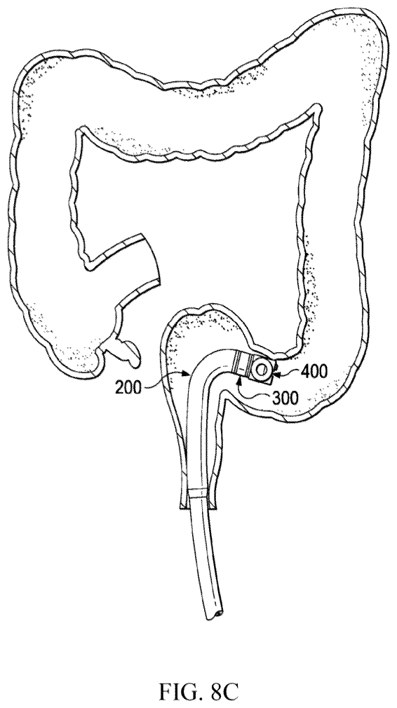

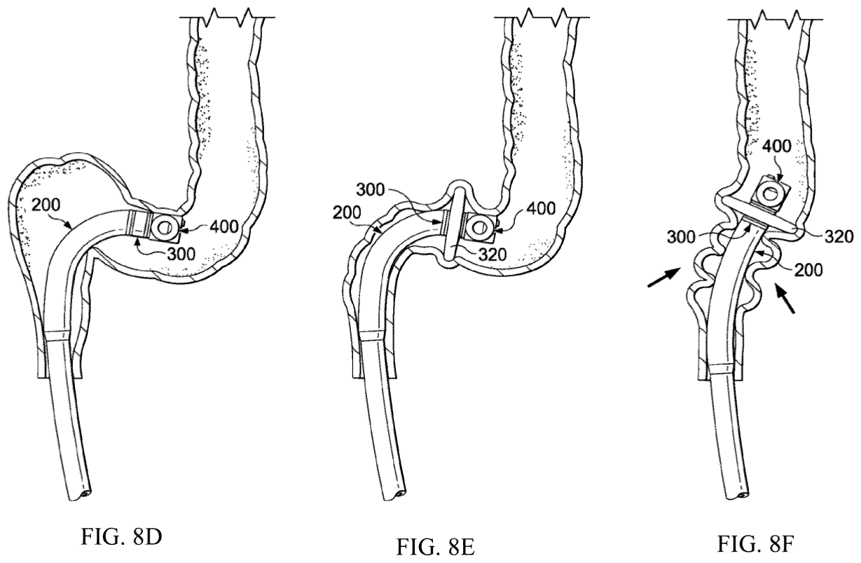

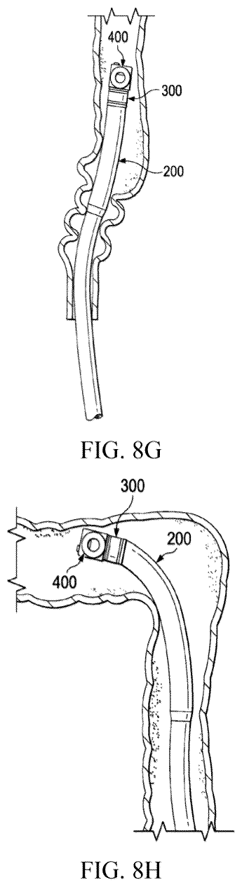

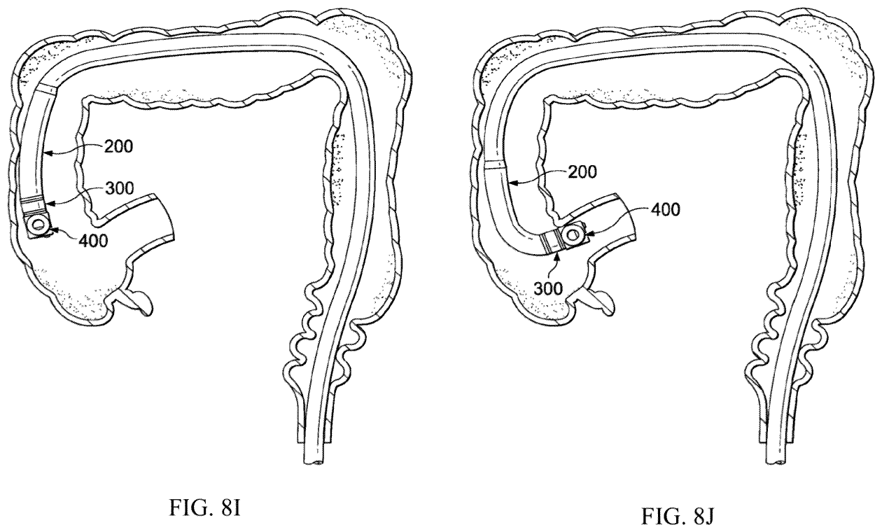

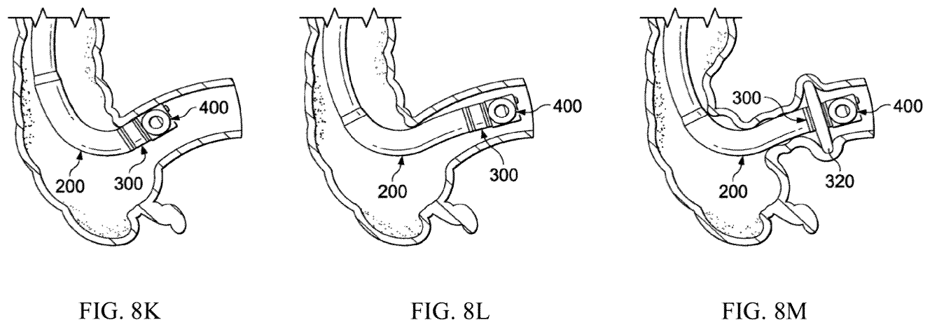

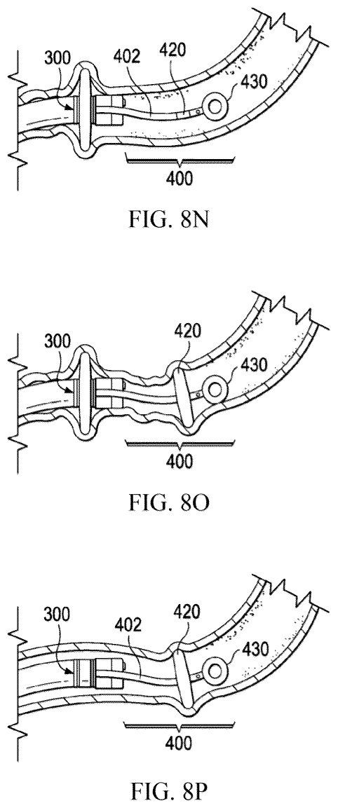

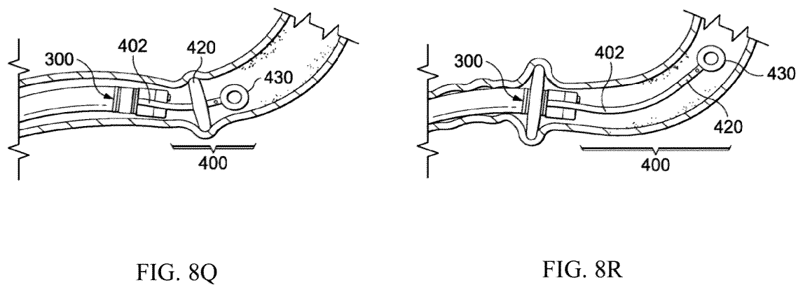

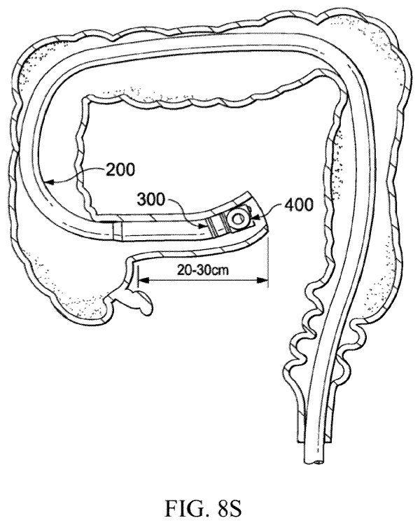

FIGS. 8A-8Q illustrate an example embodiment of a method of delivering a magnetic implant assembly. The magnetic implant assembly may be or include one or more example embodiments of the magnetic implant assembly 430 (or second magnetic implant assembly 430′) described in the present disclosure. The method may be performed using one or more example embodiments of the endoscopic anastomosis system 100 described in the present disclosure, which includes the first main body assembly 200, the head assembly 300, the second main body assembly 400, and a first magnetic implant assembly 430. The method illustrated in FIGS. 8A-8Q is an example embodiment of a method of delivering a magnetic implant assembly rectally (i.e., via the rectum and anus).

As illustrated in FIG. 8A and FIG. 8B, an example embodiment of the method includes inserting the system 100 (which includes example embodiments of the first main body assembly 200, the head assembly 300, and the second main body assembly 400, as described in the present disclosure) in the anus and through the rectum.

As illustrated in FIG. 8C and FIG. 8D, an example embodiment of the method includes bending or turning the system 100 (e.g., via the first bendable section 210 of the first main body assembly 200) and further advancing the system 100 around one or more bends or turns and into the sigmoid colon.

As illustrated in FIG. 8E, once the head assembly 300 is advanced around the bend or turn, the head assembly 300 (and first main body assembly 200) is anchored or secured to a portion of the cavity wall of the patient (e.g., by controlling/configuring the first expandable member 320 to expand radially outwards while also controlling/configuring the first pressure port (not shown here, but as described for first pressure port 332) to provide a negative pressure to encourage, bring in, suction inward, and/or collapse a portion of cavity wall of the patient towards the head assembly 300).

As illustrated in FIG. 8F, once the cavity wall of the patient is anchored or secured to the head assembly 300 and the first main body assembly 200 (as described above for FIG. 8E), the bend or turn in the colon may be straightened by pulling the system 100 (i.e., pulling the first main body assembly 200) back so as to concertina the colon.

As illustrated in FIG. 8G, once the colon has been straightened, the system 100 can be unanchored or unsecured from the cavity wall of the colon (e.g., by unexpanding or deflating the first expandable member 320 and not applying negative pressure (or applying positive pressure) by the first pressure port).

As illustrated in FIG. 8H and FIG. 8I, an example embodiment of the method includes advancing the system 100 through the descending colon, splenic flexure, transverse colon, hepatic flexure, and ascending colon until the head assembly 300 approaches the ileocecal valve.

As illustrated in FIG. 8J, FIG. 8K, and FIG. 8L, an example embodiment of the method includes bending or turning the system 100 (e.g., via the first bendable section 210 of the first main body assembly 200) and further advancing the system 100 into and through the ileocecal valve.

As illustrated in FIG. 8M, once the head assembly 300 is advanced into the ileocecal valve and to the small bowel, the head assembly 300 (and first main body assembly 200) is anchored or secured to a portion of the cavity wall (e.g., by controlling/configuring the first expandable member 320 to expand radially outwards while also controlling/configuring the first pressure port (not shown here, but as described for first pressure port 332) to provide a negative pressure to encourage, bring in, suction inward, and/or collapse a portion of cavity wall of the patient towards the head assembly 300).

As illustrated in FIG. 8N, once the cavity wall of the patient is anchored or secured to the head assembly 300 and first main body assembly 200 (as described above for FIG. 8M), the second main body assembly 400 (which includes example embodiments of the second main body 402, the second expandable member 420, and the magnetic implant assembly 430, as described in the present disclosure) is advanced forward by sliding the second main body assembly 400 forward relative to the anchored first main body assembly 200.

As illustrated in FIG. 8O, after the second main body assembly 400 has been advanced forward, the second main body assembly 400 may be anchored or secured to a portion of the cavity wall (e.g., by controlling/configuring the second expandable member 420 to expand radially outwards while also controlling/configuring the second pressure port (not shown here, but as described for second pressure port 442) to provide a negative pressure to encourage, bring in, suction inward, and/or collapse a portion of cavity wall of the patient towards the second main body 402).

As illustrated in FIG. 8P, once the cavity wall of the patient is anchored or secured to the second main body assembly 400 (as described above for FIG. 8O), the head assembly 300 and first main body assembly 200 may be unanchored or unsecured (e.g., by unexpanding or deflating the first expandable member 320 and not applying negative pressure (or applying positive pressure) by the first pressure port).

As illustrated in FIG. 8Q, after the head assembly 300 and the first main body assembly 200 have been unanchored or unsecured (as described above for FIG. 8P), the first main body assembly 200 and head assembly 300 are advanced forward toward the second expandable member 420 of the second main body assembly 400.

As illustrated in FIG. 8R, the head assembly 300 and first main body assembly 200 are then anchored or secured to a portion of the cavity wall of the patient (e.g., by controlling/configuring the first expandable member 320 to expand radially outwards while also controlling/configuring the first pressure port to provide a negative pressure to encourage, bring in, suction inward, and/or collapse a portion of cavity wall of the patient towards the head assembly 300). As explained for FIG. 8N, the second main body assembly 400 is then advanced forward.

As illustrated in FIG. 8S, the steps illustrated in FIGS. 8N-8R are repeated as necessary until the head assembly 300 approaches a distance of about 20-30 cm from the ileocecal valve. At this point, the system 100 is ready to deliver the magnetic implant assembly 430 to magnetically couple with another magnetic implant assembly 430′ delivered orally (i.e., via the mouth).

### Example Embodiments of a Method of Delivering a Magnetic Implant Assembly Orally.

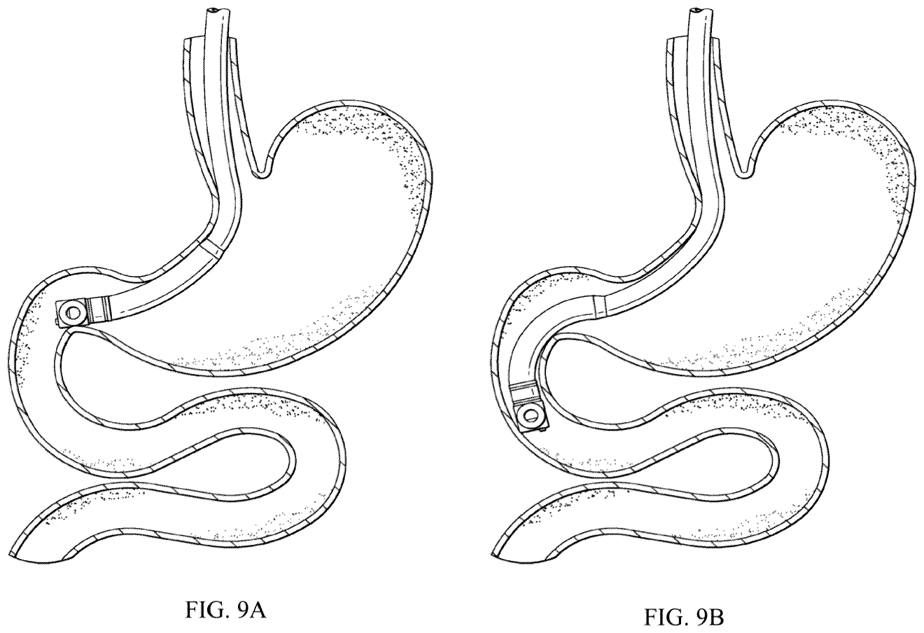

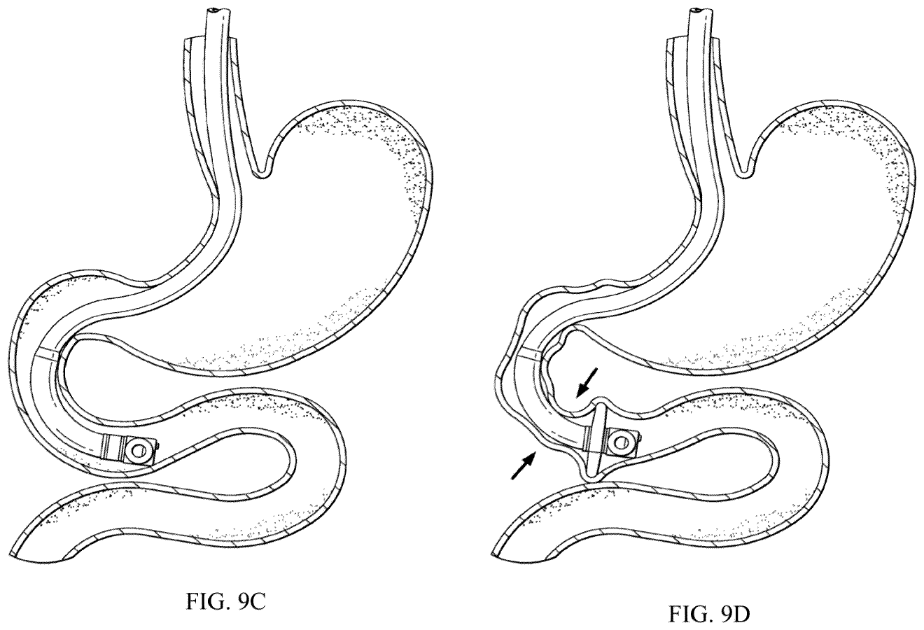

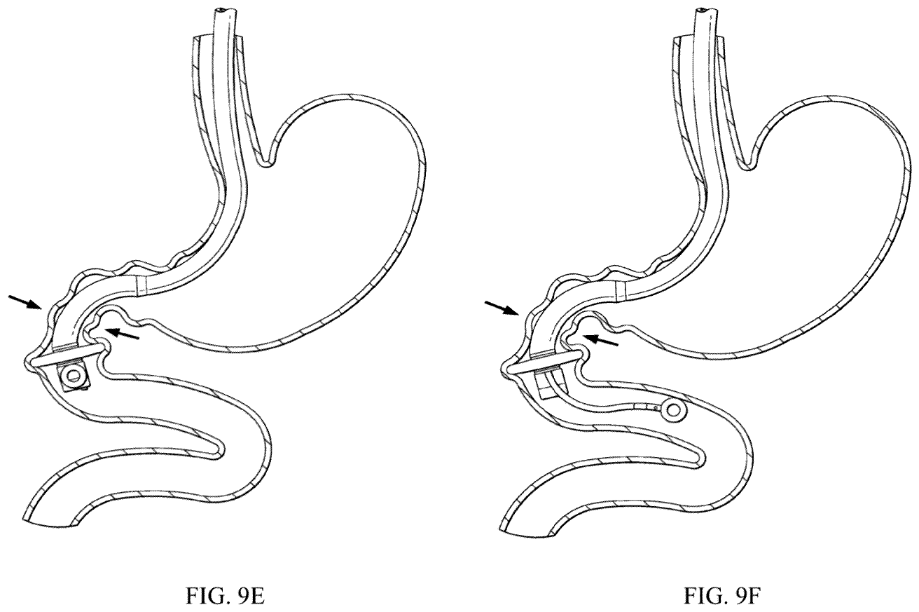

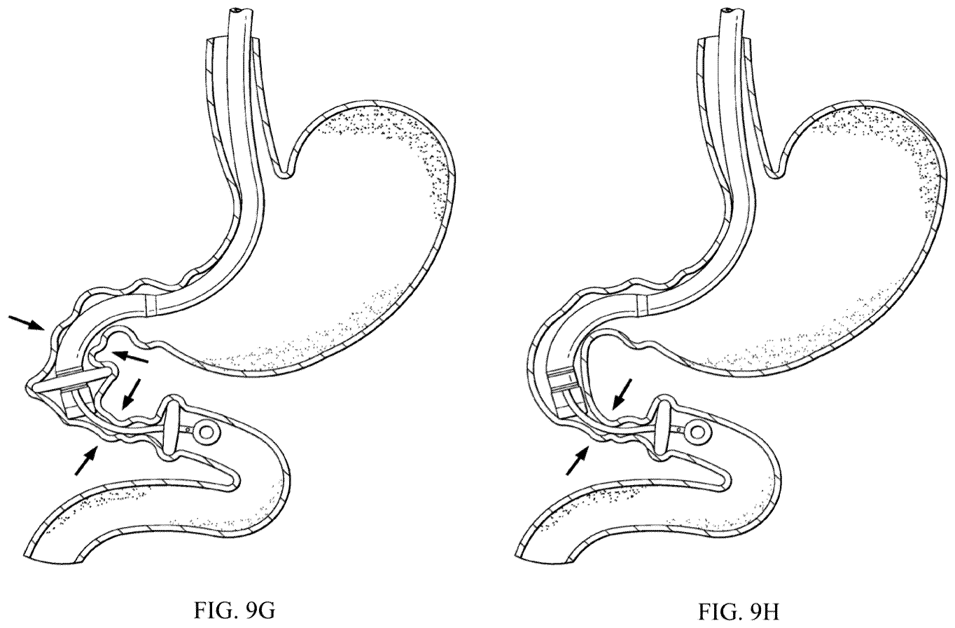

FIGS. 9A-9I illustrate another example embodiment of a method of delivering a magnetic implant assembly. The magnetic implant assembly may be or include one or more example embodiments of the magnetic implant assembly 430′ (or 430) described in the present disclosure. The method may be performed using one or more example embodiments of the endoscopic anastomosis system 100 described in the present disclosure, which includes the first main body assembly 200, the head assembly 300, the second main body assembly 400, and a second magnetic implant assembly 430′. The method illustrated in FIGS. 9A-9I is an example embodiment of a method of delivering a magnetic implant assembly orally (i.e., via the mouth and esophagus).

As illustrated in FIG. 9A and FIG. 9B, an example embodiment of the method includes inserting the system 100 (which includes example embodiments of the first main body assembly 200, the head assembly 300, and the second main body assembly 400, as described in the present disclosure) in the mouth, through the esophagus, and through the stomach. Once the system 100 has reached the pyloric sphincter, the system 100 is configured to bend or turn (e.g., via the first bendable section 210 of the first main body assembly 200) into the pyloric sphincter.

As illustrated in FIG. 9C, an example embodiment of the method includes further advancing the system 100 to the duodenum.

As illustrated in FIG. 9D, once the head assembly 300 is advanced forward, the system 100 is anchored or secured to a portion of the cavity wall of the patient (e.g., by controlling/configuring the first expandable member 320 to expand radially outwards while also controlling/configuring the first pressure port (not shown here, but as described for first pressure port 332) to provide a negative pressure to encourage, bring in, suction inward, and/or collapse a portion of cavity wall of the patient towards the head assembly 300).

As illustrated in FIG. 9E, once the cavity wall of the patient is anchored or secured to the system 100 (as described above for FIG. 9D), the bend or turn may be straightened by pulling the system 100 (i.e., pulling the first main body assembly 200) back so as to concertina the duodenum.

As illustrated in FIG. 9F, the second main body assembly 400 (which includes example embodiments of the second main body 402, the second expandable member 420, and the magnetic implant assembly 430, as described in the present disclosure) is advanced forward by sliding the second main body assembly 400 forward relative to the anchored first main body assembly 200.

As illustrated in FIG. 9G, after the second main body assembly 400 has been advanced forward, the second main body assembly 400 may be anchored or secured to a portion of the cavity wall (e.g., by controlling/configuring the second expandable member 420 to expand radially outwards while also controlling/configuring the second pressure port (not shown here, but as described for second pressure port 442) to provide a negative pressure to encourage, bring in, suction inward, and/or collapse a portion of cavity wall of the patient towards the second main body 402).

As illustrated in FIG. 9H, once the cavity wall of the patient is anchored or secured to the second main body assembly 400 (as described above for FIG. 9G), the head assembly 300 and first main body assembly 200 may be unanchored or unsecured (e.g., by unexpanding or deflating the first expandable member 320 and not applying negative pressure (or applying positive pressure) by the first pressure port).

As illustrated in FIG. 9I, after the head assembly 300 and the first main body assembly 200 have been unanchored or unsecured (as described above for FIG. 8P), the first main body assembly 200 and head assembly 300 are advanced forward toward the second expandable member 420 of the second main body assembly 400. The steps illustrated in FIGS. 9D-9I are repeated as necessary until the head assembly 300 approaches the target location/position in the distal duodenum/jejunum (i.e., the location where the first magnetic implant assembly 430 has been delivered, as described in FIGS. 8A-S). The method then includes delivering the second magnetic implant assembly 430′ to magnetically couple with magnetic implant assembly 430 delivered rectally (as described in FIGS. 8A-S).

As shown in FIGS. 10-12D, exemplary embodiments of the present application provide an endoscopic magnetic anastomosis system that may include at least one endoscope assembly 500. The endoscope assembly 500 includes an endoscope 51 and an adjustable snare mechanism. The endoscope 51 may include a flexible body or flexible tube having one or more internal channels (not shown). In an exemplary embodiment, the endoscope 51 has a first end and an opposite second end, and includes a magnetic implant assembly 511 disposed at the second end and a snare channel extending from the first end to the second end.

The adjustable snare mechanism includes a snare assembly 52 and a snare guide assembly 53. The snare assembly 52 passes through the snare channel and includes a snare catheter 521 and a snare wire 522 passing through the snare catheter 521 for selectively tightening and releasing the magnetic implant assembly 511.

In an exemplary embodiment, in an endoscopic magnetic anastomosis system, the endoscope assembly 500 includes an endoscope 51 and an adjustable snare mechanism. A first end of the endoscope 51 is a proximal end (i.e., an end located outside the human body), a second end of the endoscope 51 is a distal end (i.e., an end for entering the human body), and the second end of the endoscope 51 is provided with a magnetic implant assembly 511. The adjustable snare mechanism is used to secure the magnetic implant assembly 511 at the second end of the endoscope 51 and can also release the magnetic implant assembly 511 in order to place the magnetic implant assembly 511 into the human body.

In an exemplary embodiment, as shown in FIG. 10, the endoscopic magnetic anastomosis system may include two endoscope assemblies 500, where one endoscope assembly 500 may deploy one magnetic implant assembly 511 to the proximal jejunum through the upper gastrointestinal tract, and the other endoscope assembly 500 may deploy another magnetic implant assembly 511 to the distal ileum through the lower gastrointestinal tract. The structure of the two endoscope assemblies 500 may be the same or different, for example, the endoscope assembly 500 through the upper gastrointestinal tract may not include the outer sheath 55 (see below for detailed description).

The endoscope 51 is also provided with a snare channel, the snare channel can extend from one end of the endoscope 51 (e.g., the distal end into the human body) to the other end (e.g., the proximal end located outside the human body), the snare assembly 52 in the adjustable snare mechanism may pass through the snare channel to secure and release the magnetic implant assembly 511.

The snare assembly 52 of the adjustable snare mechanism includes a snare wire 522 and a snare catheter 521, the snare wire 522 may pass through the snare catheter 521, and both of the snare wire 522 and the snare catheter 521 may be bent and deformed to accommodate a curved lumen or intestinal tract in a human body. In an exemplary embodiment, the snare wire 522 may be made of stainless steel, the snare catheter 521 may be made of Teflon (polytetrafluoroethylene), or the materials of the snare wire 522 and the snare catheter 521 may be adjusted as desired.

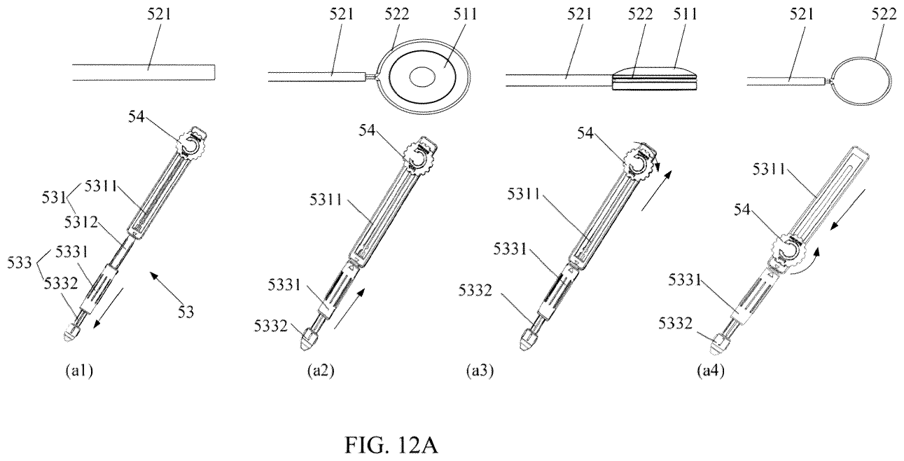

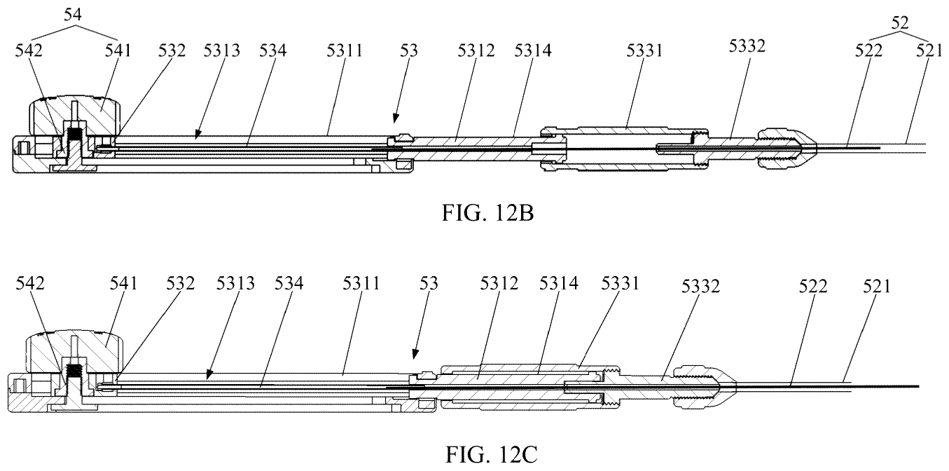

In some exemplary embodiments, as shown in FIG. 12A to FIG. 12D, the adjustable snare mechanism further comprises a snare guide assembly 53. The snare guide assembly 53 comprises a base body 531 and a motion mechanism movably installed on the base body 531. The motion mechanism is connected to and is configured to drive at least one of the snare wire 522 and the snare catheter 521, such that there is a relative movement between the snare wire 522 and the snare catheter 521 to allow the snare wire 522 to selectively extend and retract relative to the snare catheter 521. In an exemplary embodiment, the snare guide assembly 53 may include a first movable member 532, and a second movable member 533. Both the first movable member 532 and the second movable member 533 are movably mounted on the base body 531. The first movable member 532 is connected to the snare wire 522 and arranged to drive the snare wire 522 to move, and the second movable member 533 is connected to the snare catheter 521 and arranged to drive the snare catheter 521 to move such that when at least one of the first movable member 532 and the second movable member 533 is moved, there is the relative movement between the snare wire 522 and the snare catheter 521 that allows the snare wire 522 to extend and retract relative to the snare catheter 521.

The adjustable snare mechanism includes a snare guide assembly 53 in addition to the snare assembly 52. In the snare guide assembly 53, both the first movable member 532 and the second movable member 533 are movably mounted on the base body 531, and the first movable member 532 is connected with the snare wire 522, and the second movable member 533 is connected with the snare catheter 521, so that the first movable member 532 can drive the snare wire 522 to move when the first movable member moves, and the second movable member 533 can drive the snare catheter 521 to move when the second movable member moves. Upon movement of at least one of the first movable member 532 and the second movable member 533, there is a relative movement between the snare wire 522 and the snare catheter 521 such that the snare wire 522 can be retracted into and extended from the snare catheter 521. During use, the snare wire 522 may first be retracted into the snare catheter 521 so that the snare assembly 52 passes through the snare channel of the endoscope 51 (as shown in the state (a1) in FIG. 12A and FIG. 12B); then, the snare wire 522 may be moved to extend out of the snare catheter 521 in order to install the magnetic implant assembly 511 into a portion of the snare wire 522 extending outside the snare catheter 521 (as shown in state (a2) in FIG. 12A); after installation of the magnetic implant assembly 511, the snare wire 522 may be retracted further into the snare catheter 521 so that the snare wire 522 tightens the magnetic implant assembly 511 (as shown in state (a3) in FIG. 12A and FIG. 12C); after the magnetic implant assembly 511 reaches a suitable position (set position) in the human body, the snare wire 522 may be further extended out of the snare catheter 521 to release the magnetic implant assembly 511, releasing the magnetic implant assembly 511 in this suitable position (as shown in state (a4) in FIG. 12A and FIG. 12D).

In some exemplary embodiments, as shown in FIGS. 12A to 12D, the base body 531 is provided with a first slide rail 5313 and a second slide rail 5314 extending along the length direction of the base body 531, the first movable member 532 is disposed to be slidable along the first slide rail 5313, and the second movable member 533 is disposed to be slidable along the second slide rail 5314.

The base body 531 of the snare guide assembly 53 is provided with a first slide rail 5313 and a second slide rail 5314 extending along the length direction thereof, and the first movable member 532 and the second movable member 533 can slide along the first slide rail 5313 and the second slide rail 5314 respectively, thereby driving the snare wire 522 and the snare catheter 521 to move, so that the snare wire 522 can be retracted into and extended out of the snare catheter 521.

In some exemplary embodiments, as shown in FIGS. 12B to 12D, the first slide rail 5313 and the second slide rail 5314 are sequentially disposed along the length direction of the base body 531, and one end of the second movable member 533 away from the first movable member 532 is connected to the first end (i.e., the proximal end) of the snare catheter 521. The snare guide assembly 53 also includes a connecting member 534, one end of the snare wire 522 passes out of the first end of the snare catheter 521 and is fixedly connected with one end of the connecting member 534, and the other end of the connecting member 534 is connected with the first movable member 532.

As shown in FIGS. 12B to 12D, both the first slide rail 5313 and the second slide rail 5314 extend along the length direction of the base body 531, and the first slide rail 5313 is located on the left side of the second slide rail 5314, the right end of the second movable member 533 is connected to the first end of the snare catheter 521, and the second movable member 533 can drive the snare catheter 521 to move; the end portion of the snare wire 522 passes out of the first end of the snare catheter 521, and is connected with the first movable member 532 through the connecting member 534, so that the first movable member 532 can drive the snare wire 522 to move through the connecting member 534. In an exemplary embodiment, the connector 534 may be a connecting crimp rod. For example, the connecting member 534 may be a hollow connecting tube, and the end portion of the snare wire 522 may extend into the connecting tube after passing out of the first end of the snare catheter 521, and the connection between the connecting member 534 and the snare wire 522 can be achieved by crimping.

In some exemplary embodiments, as shown in FIGS. 12A to 12D, the base body 531 includes a first base body portion 5311 and a second base body portion 5312 connected to each other, the first slide rail 5313 includes a slide slot provided on the first base body portion 5311, and the first movable member 532 includes a slider cooperating with the slide slot; the second slide rail 5314 is provided on the outer surface of the second base body portion 5312, and the second movable member 533 is sleeved on the outer surface of the second base body portion 5312.

The base body 531 includes a first base body portion 5311 and a second base body portion 5312, and the first base body portion 5311 is provided with a slide slot to form a first slide rail 5313, and a slider of the first movable member 532 can cooperate with the slide slot so that the first movable member 532 slides along the first slide rail 5313.

The outer surface of the second base body portion 5312 may form the second slide rail 5314, or the outer surface of the second base body portion 5312 may be provided with the second slide rail 5314, the second movable member 533 is sleeved on the outer surface of the second base body portion 5312, and the second movable member 533 is in sliding fit with the second slide rail 5314 so that the second movable member 533 slides along the second slide rail 5314. In an exemplary embodiment, the second movable member 533 may include a sheath segment 5331 and a fixed segment 5332, the sheath segment 5331 may be sleeved on an outer surface of the second base body portion 5312 and may be slidable relative to the second base body portion 5312; the fixed segment 5332 can be fixedly connected with the sleeve section 5331, and the fixed segment 5332 is connected with the snare catheter 521 to realize the connection between the second movable member 533 and the snare catheter 521.

The connecting member 534 may be disposed in the slide slot of the first base body portion 5311, and the end portion of the snare wire 522 may be connected to the connecting member 534 after passing through the snare catheter 521, the second movable member 533, and the second base body portion 5312.

In some exemplary embodiments, as shown in FIGS. 12A-12D, the snare guide assembly 53 further includes a releasable locking mechanism 54 mounted to the first movable member 532, the releasable locking mechanism 54 is arranged to be in locking fit with the base body 531 to secure the first movable member 532 to the base body 531, and to disengage from being in locking fit with the base body 531 to enable the first movable member 532 to move relative to the base body 531.

In the snare guide assembly 53, the releasable locking mechanism 54 has a locked state and a released state. As shown in the state (a3) of FIG. 12A, when the releasable locking mechanism 54 is in the locked state, it can be in locking fit with the base body 531 (such as the first base body portion 5311), and at this time, the releasable locking mechanism 54 is fixed with the base body 531, so that the first movable member 532 is fixed with the base body 531 to prevent the snare wire 522 from moving, and so that the snare wire 522 can be maintained in the state of tightening the magnetic implant assembly 511 to firmly secure the magnetic implant assembly 511. As shown in the states (a1), (a2) and (a4) in FIG. 12A, when the releasable locking mechanism 54 is in the released state, the releasable locking mechanism may be disengaged from being in locking fit with the base body 531 (e.g., the first base body portion 5311), and at this time, the releasable locking mechanism 54 is disengaged from being in fixation with the base body 531, so that the first movable member 532 can move relative to the base body 531, allowing the first movable member 532 to drive the snare wire 522 to move, so that the snare wire 522 can be retracted into the snare catheter 521, and the magnetic implant assembly 511 can be installed and released.

In some exemplary embodiments, as shown in FIGS. 12A to 12D, the releasable locking mechanism 54 includes a rotary locking mechanism including a knob 541 and a clamping member 542, the knob 541 is connected to the clamping member 542, and the knob 541 is configured to be rotatable in two opposite directions to drive the clamping member 542 to clamp and release the base body 531 accordingly.

The releasable locking mechanism 54 may be a rotary locking mechanism mounted to the first movable member 532, and the knob 541 thereof may be connected to the clamping member 542, and when the knob 541 is rotated, the knob can drive the clamping member 542 to move. Specifically, when the knob 541 is rotated in one direction (for example, the counterclockwise direction or the clockwise direction indicated by the curved arrow in the state (a4) in FIG. 12A), the knob 541 can drive the clamping member 542 to release the base body 531 (for example, the first base body portion 5311) so that the releasable locking mechanism 54 and the first movable member 532 move relative to the base body 531; when the knob 541 is rotated in the opposite direction (e.g., the clockwise direction or the counterclockwise direction indicated by the curved arrow in the state (a3) of FIG. 12A), the knob 541 can drive the clamping member 542 to clamp the base body 531 (e.g., the first base body portion 5311), so that the releasable locking mechanism 54 and the first movable member 532 are clamped and fixed to the base body 531, preventing the releasable locking mechanism 54 and the first movable member 532 from moving relative to the base body 531.

During use, as shown in the state (a1) of FIG. 12A, the second movable member 533 can be slid along the base body 531 (as shown in the direction shown by the straight arrow in the state (a1) of FIG. 12A) first, so that the second movable member 533 moves in a direction away from the first base body portion 5311 and drives the snare catheter 521 to move, so that the snare wire 522 can be gradually retracted into the snare catheter 521, which facilitates the snare assembly 52 to pass through the snare channel of the endoscope 51; then, as shown in the state (a2) of FIG. 12A, the second movable member 533 can be slid in the opposite direction (as shown in the direction shown by the straight arrow in the state (a2) of FIG. 12A) to move the second movable member 533 in the direction close to the first base body portion 5311 and drive the snare catheter 521 to move so that the snare wire 522 can gradually extend out of the snare catheter 521, at this time, the snare wire 522 is in a released state, so as to install the magnetic implant assembly 511 into the portion of the snare wire 522 extending out of the snare catheter 521; after installing the magnetic implant assembly 511, as shown in the state (a3) in FIG. 12A, the first movable member 532 can be slid along the base body 531 (as shown in the direction indicated by the straight arrow in the state (a3) in FIG. 12A) to move the first movable member 532 in the direction away from the second base body portion 5312, and drive the snare wire 522 to move so that the snare wire 522 can gradually retract into the snare catheter 521 so that the snare wire 522 tightens the magnetic implant assembly 511, and the knob 541 of the releasable locking mechanism 54 is rotated (in the direction indicated by the curve arrow in the state (a 3) in FIG. 12A) to lock the first movable member 532 with the base body 531, the snare wire 522 always remains tightened and fixes the state of the magnetic implant assembly 511 during intubation of the endoscope 51 and delivery of the snare guide assembly 53. When the magnetic implant assembly 511 needs to be released after reaching a suitable position in the human body, as shown in the state (a4) of FIG. 12A, the knob 541 of the releasable locking mechanism 54 can be rotated in an opposite direction (as shown by the curved arrow in the state (a4) of FIG. 12A) to unlock the first movable member 532 from the base body 531, then the first movable member 532 can be moved in a direction close to the second base body portion 5312 (as indicated by the straight arrow in state (a4) in FIG. 12A), and drive the snare wire 522 to move so that the snare wire 522 can gradually extend out of the snare catheter 521 to release the magnetic implant assembly 511. At this time, the magnetic implant assembly 511 can be released in the appropriate position. When the snare wire 522 releases the magnetic implant assembly 511 to release the magnetic implant assembly 511, the connecting member 534 may remain within the snare catheter 521 (as shown in FIG. 12D), preventing the connecting member 534 from pinching any tissue in the human body.

In some exemplary embodiments, the snare guide assembly 53 further includes a unlockable locking device mounted to the second movable member 533, the unlockable locking device is arranged to be in locking fit with the base body 531 to secure the second movable member 533 to the base body 531, and to disengage from being in locking fit with the base body 531 to enable the second movable member 533 to move relative to the base body 531.

In the snare guide assembly 53, the unlockable locking device may have a locked state and a released state. When the unlockable locking device is in the locking state, the unlockable locking device can be in locking fit with the base body 531 (such as the second base body portion 5312), and at this time, the unlockable locking device is fixed with the base body 531, so that the second movable member 533 is fixed with the base body 531 to prevent the snare catheter 521 from moving; when the unlockable locking device is in the released state, the unlockable locking device can be disengaged from being in locking fit with the base body 531 (such as the second base body portion 5312), and at this time, the unlockable locking device is disengaged from being in fixation with the base body 531, so that the second movable member 533 can move relative to the base body 531, so that the second movable member 533 drives the snare catheter 521 to move. In an exemplary embodiment, the structure of the unlockable locking device may be the same as or different from the structure of the releasable locking mechanism 54.

It should be understood that the second movable member 533 and the base body 531 may be locked or unlocked by the unlockable locking device, or may be locked or released by other means. For example, in other exemplary embodiments, as shown in FIGS. 12A to 12D, the second movable member 533 is in friction fit with the base body 531, so that the second movable member 533 can remain fixed relative to the base body 531 under the action of the frictional force of the base body 531, and can also be moved relative to the base body 531 against the frictional force of the base body 531. Specifically, in a case where the second movable member 533 is sleeved over the second base body portion 5312 of the base body 531, the inner diameter of the second movable member 533 may be smaller than the outer diameter of the second base body portion 5312, so that the second movable member 533 is in friction fit (e.g., interference fit) with the second base body portion 5312, and therefore, there is a frictional force between the second movable member 533 and the second base body portion 5312 due to extrusion, and the second movable member 533 may be fixed relative to the second base body portion 5312 under the action of the frictional force; the second movable member 533 may also overcome the frictional force and slide relative to the second base body portion 5312 under the action of an external force (e.g., under the urging action of a human hand).

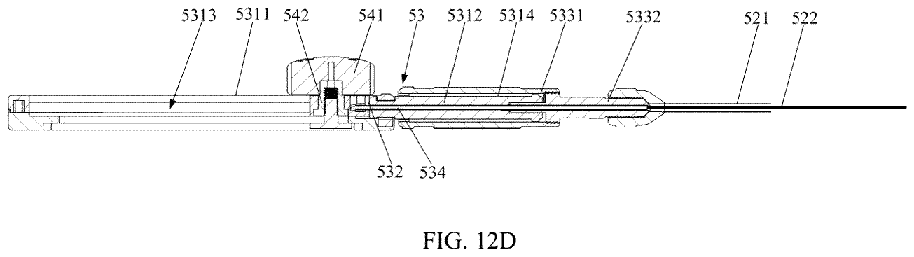

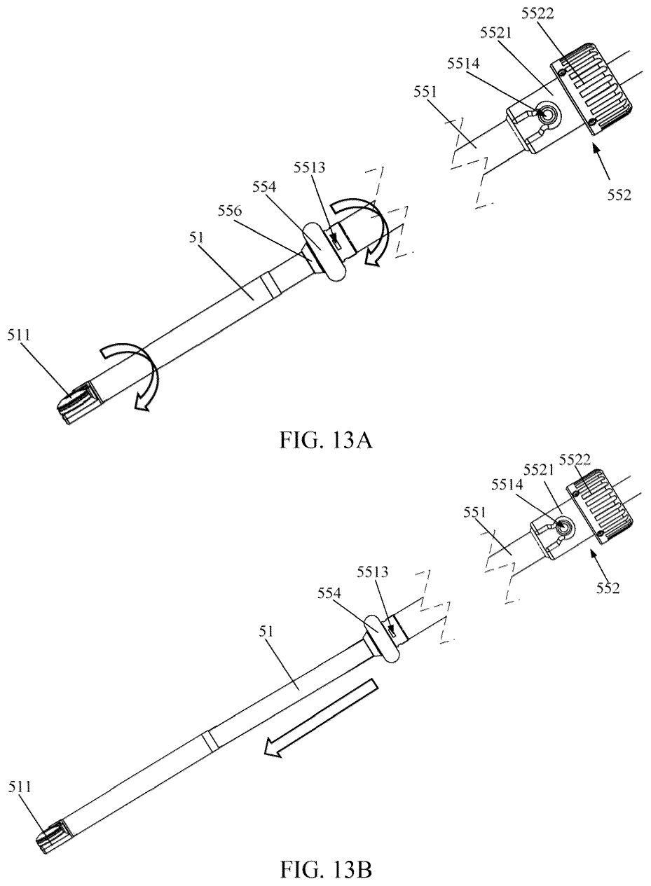

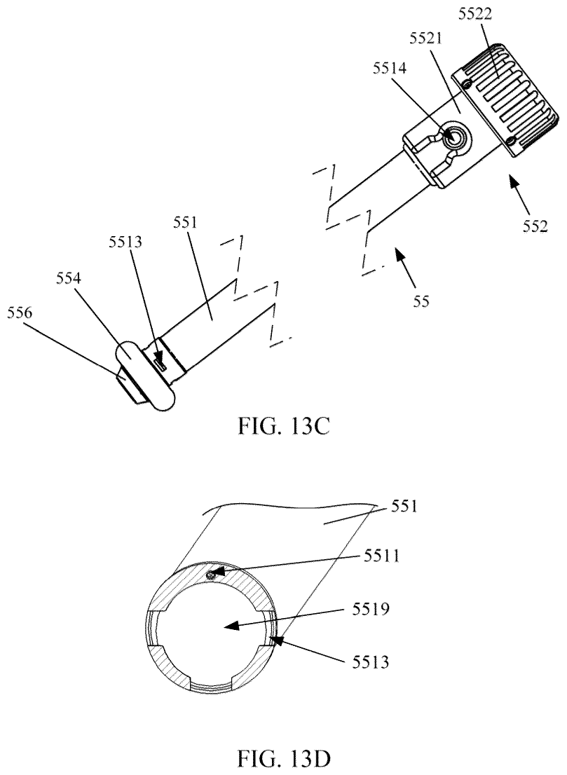

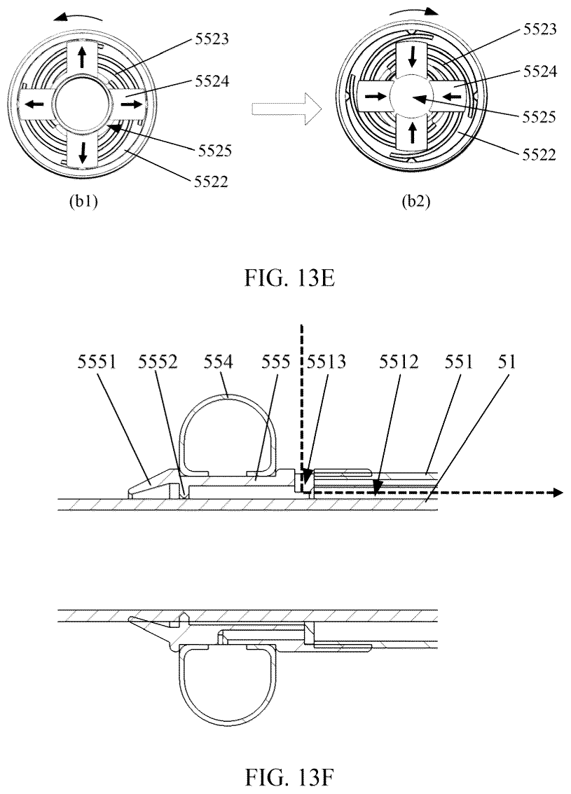

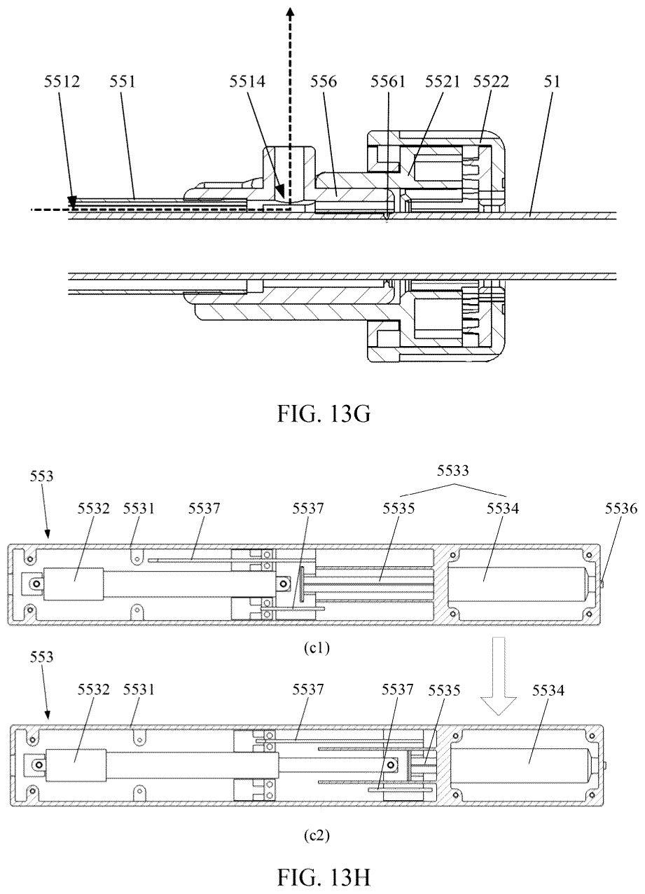

In some exemplary embodiments, as shown in FIGS. 13A-13G, the endoscope assembly 500 further includes an outer sheath 55, the outer sheath 55 may include a body tube having a first end (i.e., a proximal end) and an opposite second end (i.e., a distal end), and a tube-locking mechanism 552 mounted at the first end of the body tube, the body tube has an endoscope channel 5519 extending from the first end to the second end of the body tube for passage of the endoscope 51, and the tube-locking mechanism 552 is arranged to be in locking fit with the endoscope 51 to secure the endoscope 51 to the body tube and also disengage from being in locking fit with the endoscope 51 to allow the endoscope 51 to slide and rotate relative to the body tube.

In the outer sheath 55, the body tube thereof is provided with an endoscope channel 5519, the endoscope 51 can pass through the endoscope channel 5519, and the endoscope 51 and the endoscope channel 5519 can be in gap fit so that the endoscope 51 can slide or rotate within the endoscope channel 5519. The tube-locking mechanism 552 may be installed at one end of the body tube (for example, the proximal end located outside the human body), and the tube-locking mechanism 552 may have a locked state and a released state. As shown in FIG. 13A, when the tube-locking mechanism 552 is in the locked state, it can be in locking fit with the endoscope 51, and at this time, the tube-locking mechanism 552 is fixed with the endoscope 51, so that the body tube and the endoscope 51 are fixed to prevent the body tube and the endoscope 51 from moving relatively, so as to drive the endoscope 51 to move or rotate by applying a thrust or torque force to the body tube, so that the endoscope 51 and the body tube are inserted into the human body together; as shown in FIG. 13B, when the tube-locking mechanism 552 is in the released state, the tube-locking mechanism can be disengaged from being in locking fit with the endoscope 51, and at this time, the tube-locking mechanism 552 is unfixed from the endoscope 51 so that the endoscope 51 can be moved relative to the body tube so as to move or rotate the endoscope 51 by applying a thrust or torque force to the endoscope 51 so that the endoscope 51 can be further inserted into the human body relative to the body tube.

By providing the tube-locking mechanism 552, the outer sheath 55 can be engaged to the endoscope 51 by the tube-locking mechanism 552 in the locked state so as to transmit a driving torque applied to the outer sheath 55 to the endoscope 51, and a 1:1 torque transmission from the outer sheath 55 to the endoscope 51 can be realized so that the outer sheath 55 and the endoscope 51 are moved or rotated together to be inserted into the human body; the tube-locking mechanism 552 in the released state may be used to disengage the outer sheath 55 from the endoscope 51 such that the endoscope 51 may move or rotate relative to the outer sheath 55.

In some exemplary embodiments, as shown in FIGS. 13A-13C and 13E, the tube-locking mechanism 552 includes a locking seat 5521, a rotatable member 5522, and a plurality of locking blocks 5524. The locking seat 5521 is mounted at the first end of the body tube. The rotatable member 5522 is rotatably mounted to the locking seat 5521, and is provided with a spiral driving part 5523, the spiral center line of the spiral driving part 5523 coincides with the rotation center line of the rotatable member 5522. The plurality of locking blocks 5524 are movably mounted to the locking seat 5521, and are configured to be in driving fit with the spiral driving part so as to be translatable in the radial direction of the spiral driving part 5523. End portions of the plurality of locking blocks 5524 adjacent to each other collectively define an opening 5525 for the endoscope 51 to pass through. The size of the opening 5525 is changed with the radial translation of the locking blocks 5524.

The plurality of locking blocks 5524 are configured such that when the rotatable member 5522 is rotated in the first direction (as shown by the curved arrow in the state (b2) of FIG. 13E), the plurality of locking blocks 5524 are translated radially inward toward the spiral center line of the spiral driving part 5523 (as shown by the straight arrow in the state (b2) of FIG. 13E) to clamping the endoscope 51 by narrowing the opening 5525. The driving fit of the plurality of locking blocks 5524 with the spiral driving part 5523 also causes the plurality of locking blocks 5524 to be translated radially outward away from the spiral center line of the spiral driving part 5523 (as shown by the straight arrow in the state (b1) in FIG. 13E) when the rotatable member 5522 is rotated in a second direction opposite to the first direction (as shown by the curved arrow in the state (b1) in FIG. 13E) to enlarge the opening 5525 and release the endoscope 51.

In the tube-locking mechanism 552, the rotatable member 5522 is rotatably mounted to the locking seat 5521, the plurality of locking blocks 5524 are translationally mounted to the locking seat 5521, and the locking seat 5521 is mounted to the first end of the body tube, so that the tube-locking mechanism 552 is mounted to the first end of the body tube. The rotatable member 5522 is provided with a spiral driving part 5523, the spiral center line of the spiral driving part 5523 coincides with the rotating center line of the rotatable member 5522, and the plurality of locking blocks 5524 are in driving fit with the spiral driving part 5523, and can be translated along a side close to or away from the spiral center line of the spiral driving part 5523 under the driving of the rotatable member 5522. In an exemplary embodiment, when the rotatable member 5522 is rotated in a first direction (clockwise or counterclockwise direction as shown by the curved arrow in the state (b2) in FIG. 13E), the plurality of locking blocks 5524 can be translated radially inward toward the spiral center line of the spiral driving part 5523 under the push of the spiral driving part 5523 of the rotatable member 5522 (as shown by the straight arrow in the state (b2) in FIG. 13E), so that the cross-sectional area of the opening 5525 formed between the plurality of locking blocks 5524 for the endoscope 51 to pass through is reduced, so that the plurality of locking blocks 5524 clamp the endoscope 51, the locking fit is achieved between the tube-locking mechanism 552 and the endoscope 51. When the rotatable member 5522 is rotated in a second direction opposite to the first direction (counterclockwise or clockwise direction as indicated by the curved arrow in the state (b1) in FIG. 13E), the plurality of locking blocks 5524 can be translated radially outward away from the spiral center line of the spiral driving part 5523 under the push of the spiral driving part 5523 of the rotatable member 5522 (as indicated by the straight arrow in the state (b1) in FIG. 13E), so that the cross-sectional area of the opening 5525 formed between the plurality of locking blocks 5524 through which the endoscope 51 passes is increased, so that the plurality of locking blocks 5524 releases the endoscope 51 and it is achieved that the tube-locking mechanism 552 is disengaged from being in locking fit with the endoscope 51.

In some exemplary embodiments, as shown in FIG. 13E, at least two locking blocks 5524 may be provided, such as four, etc. The plurality of locking blocks 5524 may be uniformly arranged along the circumferential direction of the spiral driving part 5523.

In some exemplary embodiments, as shown in FIGS. 13A to 13C, the outer surface of the rotatable member 5522 is provided with a non-slip structure (e.g., a non-slip protrusion) to rotate the rotatable member 5522.

In some exemplary embodiments, as shown in FIGS. 13A-13C and 13F, the outer sheath 55 further includes an expandable member 554, the expandable member 554 is sleeved over an outer surface of the body tube at a second end of the body tube, the body tube provided with a gas channel 5511 extending between an inner surface and an outer surface of the body tube in an axial direction of the body tube, the gas channel 5511 is in communication with the expandable member 554 at the second end of the body tube, and the gas channel 5511 is in communication with an external pressure source 553 at the first end of the body tube so that the expandable member 554 expands outwardly in a radial direction of the body tube from a non-expanded state to an expanded state or retracts from the expanded state to the non-expanded state in response to action of the external pressure source 553.

In the outer sheath 55, the expandable member 554 is sleeved over the outer surface of the body tube at the second end of the body tube (e.g., the distal end into the human body). The expandable member 554 may include a balloon, and the body tube is provided with a gas channel 5511 that communicates the external pressure source 553 with the expandable member 554 so that the external pressure source 553 supplies air to the expandable member 554 through the gas channel 5511 so that the expandable member 554 can be expanded outward along the radial direction of the body tube to the lumen wall of the human body so that the expandable member 554 is anchored or gripped to the lumen wall of the human body, achieving fixation of the outer sheath 55 to the human body.

The external pressure source 553 may not only provide positive pressure to supply air to the expandable member 554 to bring the expandable member 554 in the expanded configuration; the external pressure source 553 may also provide negative pressure to extract gas from the expandable member 554 such that the expandable member 554 may contract inwardly along the radial direction of the body tube while the expandable member 554 is in a normal or unexpanded configuration.

In some exemplary embodiments, the endoscope assembly 500 further includes the external pressure source 553 and the external pressure source 553 is configured to provide a fixed volume of gas to the expandable member 554 to expand the expandable member 554 outwardly in a radial direction of the body tube.

When the expandable member 554 is expanded, the external pressure source 553 may provide a fixed volume of gas to the expandable member 554 to control the expanded volume of the expandable member 554. Compared to providing a fixed pressure gas to control the expansion of the expandable member 554 through pressure, the embodiments of the present application provides an external pressure source 553, making the expansion control of the expandable member 554 simpler, easier, and safer (such as when the expandable member 554 leaks gas).

In some exemplary embodiments, as shown in FIG. 13H, the external pressure source 553 includes a housing 5531, a first linear actuator 5532 mounted within the housing 5531, a syringe 5533 mounted within the housing 5531, and two position sensors 5537 mounted within the housing 5531. The first linear actuator 5532 has a drive end configured to move between a first position (as shown in state (c2) in FIG. 13H) and a second position (as shown in state (c1) in FIG. 13H). The syringe 5533 includes a syringe barrel 5534 and a syringe plunger 5535 movably mounted to the syringe barrel 5534, the syringe barrel 5534 has an injection port 5536 in communication with the gas channel 5511, the drive end of the first linear actuator 5532 is connected to the syringe plunger 5535 and configured to drive the syringe plunger 5535 to reciprocate relative to the syringe barrel 5534 to supply gas to the expandable member 554 or draw gas from the expandable member 554 through the injection port 5536. Two position sensors 5537 are provided for detecting the position of the drive end of the first linear actuator 5532.

In the external pressure source 553, the drive end of the first linear actuator 5532 can reciprocate and telescopically move, and can drive the syringe plunger 5535 of the syringe 5533 connected thereto to reciprocate. When the syringe plunger 5535 of the syringe 5533 is moved in one direction (e.g., to the right to transition from the state (c1) in FIG. 13H to the state (c2) in FIG. 13H), the syringe barrel 5534 of the syringe 5533 can supply gas to the expandable member 554 through the injection port 5536 so that the expandable member 554 can be expanded outward in a radial direction of the body tube; when the syringe plunger 5535 of the syringe 5533 is moved in the opposite direction (e.g., to the left to transition from the state (c2) in FIG. 13H to the state (c1) in FIG. 13H), the syringe barrel 5534 of the syringe 5533 may draw gas from the expandable member 554 through the injection port 5536 so that the expandable member 554 may be retracted inward in a radial direction of the body tube.

The two position sensors 5537 may be used to detect the position of the drive end of the first linear actuator 5532, causing the drive end of the first linear actuator 5532 to move between the first position and the second position. For example, when the drive end of the first linear actuator 5532 is moved to the first position (in which the syringe 5533 supplies a fixed volume of gas to the expandable member 554), a sensor is actuated, and then the drive end of the first linear actuator 5532 can be controlled to stop moving and stop driving the syringe plunger 5535 to continue inflating the expandable member 554; when the drive end of the first linear actuator 5532 is moved to the second position (during which the syringe 5533 draws air from the expandable member 554), another sensor is actuated, and the drive end of the first linear actuator 5532 can be controlled to stop moving and return to the original position.

In some exemplary embodiments, as shown in FIGS. 13A-13C, 13F-13G, the outer sheath 55 further includes a first seal 556 and a second seal 555, the first seal 556 is disposed at a first end of the body tube (e.g., a proximal end of the body tube outside the human body) between the body tube and the outer surface of the endoscope 51, and the second seal 555 is disposed at a second end of the body tube (e.g., a distal end of the body tube into the human body) between the body tube and the outer surface of the endoscope 51.

In some exemplary embodiments, as shown in FIGS. 13A to 13D, 13F to 13G, the gap between the inner surface of the body tube and the outer surface of the endoscope 51 forms a suction channel 5512, the body tube has one or more suction openings 5513 provided in the circumferential direction of the body tube at the second end of the body tube and a suction connector 5514 provided at the first end of the body tube, the suction openings 5513 and the suction connector 5514 are located between the second seal 555 and the first seal 556, two ends of the suction channel 5512 is in communication with the suction opening 5513 and the suction connector 5514, respectively. The suction connector 5514 is set to connect with a suction apparatus used to provide negative pressure. In an exemplary embodiment, the suction connector 5514 may be a suction interface.

Two ends of the suction channel 5512 formed by the gap between the inner surface of the body tube and the outer surface of the endoscope 51 can be sealed by the first seal 556 and the second seal 555, respectively, and the suction opening 5513 and the suction connector 5514 located between the first seal 556 and the second seal 555 are opened on the body tube, so that the suction opening 5513 can be in communication with the suction channel 5512 on the one hand, and can be in communication with the lumen or intestinal tract of the human body when one end of the endoscope 51 and the outer sheath 55 is inserted into the lumen or intestinal tract of the human body on the other hand; the suction connector 5514 is in communication with the suction channel 5512 on the one hand, and may be is in communication with the suction apparatus on the other hand, so that the suction apparatus can extract gas from the lumen or intestinal tract of the human body through the suction connector 5514, the suction channel 5512, and the suction opening 5513 (the flow direction of the gas is shown by dashed arrows in FIGS. 13F and 13G), so that the lumen wall of the lumen or intestinal tract is tightly adsorbed to the outer surface of the body tube under the action of negative pressure, to realize the fixation of the body tube of the outer sheath 55 to the lumen wall of the human body.

The expandable member 554 is expanded and anchored to the lumen wall of the human body and the lumen wall of the human body is adsorbed and fixed to the body tube of the outer sheath 55 by suction negative pressure, thereby enhancing the fixing effect of the outer sheath 55 and the lumen wall of the human body, and reducing the size of the expandable member 554. Compared to the expandable member 554 fixed by anchoring only (e.g., a balloon having an expanded outer diameter of up to 60 mm), the expandable member 554 (e.g., a balloon having an expanded outer diameter of up to 30 mm) according to an embodiment of the present application is small in size and provides a secure anchoring without slipping and over-inflating in the lumen or intestinal tract of the human body. Furthermore, using the expandable member 554 according to an embodiment of the present application can save installation time and cost as compared to the expandable member 554 that is only fixed by anchoring.

In some exemplary embodiments, as shown in FIGS. 13F and 13G, the second seal 555 is located on a side close to the suction opening 5513 and the first seal 556 is located on a side close to the suction connector 5514. In an exemplary embodiment, the second seal 555 and the suction opening 5513 may be located at one end of the body tube (e.g., the distal end of the body tube into the human body), and the first seal 556 and the suction connector 5514 may be located at the other end of the body tube (e.g., the proximal end of the body tube outside the human body).

In some exemplary embodiments, as shown in FIG. 13F, a side of the second seal 555 away from the first seal 556 is provided with a conical part 5551, and the tapered end of the conical part 5551 away from the first seal 556 is in seal fit with the outer surface of the endoscope 51; the second seal 555 is further provided with a second sealing rib 5552, the second sealing rib 5552 is closer to the first seal 556 than the conical part 5551, and the second sealing rib 5552 is in seal fit with the outer surface of the endoscope 51.

The side of the second seal 555 away from the first seal 556 is provided with a conical part 5551, and the outer diameter of the conical part 5551 gradually decreases along the direction away from the first seal 556, so as to avoid the lumen wall, intestinal wall or mucosal folds of the human body being caught by the outer sheath 55 during the advancement of the endoscope 51 and the outer sheath 55 in the human body.

The second seal 555 includes not only the conical part 5551, but also a second sealing rib 5552 located at one end of the conical part 5551 close to the first seal 556. When the second seal 555 is in seal fit with the outer surface of the endoscope 51, one end of the conical part 5551 away from the first seal 556 can be in seal fit with the outer surface of the endoscope 51 on the one hand, and the second sealing rib 5552 can be in seal fit with the outer surface of the endoscope 51 on the other hand, thereby achieving double sealing with good sealing effect, so as to block the body fluid in the human body from entering the suction channel 5512 between the inner surface of the body tube of the outer sheath 55 and the outer surface of the endoscope 51.

In some exemplary embodiments, as shown in FIG. 13G, the first seal 556 is provided with a first sealing rib 5561, the first sealing rib 5561 is in seal fit with the outer surface of the endoscope 51 to ensure the sealing effect between the inner surface of the body tube of the outer sheath 55 and the outer surface of the endoscope 51 to prevent the body fluid in the human body from leaking to the outside of the body, and can maintain the negative pressure around the suction opening 5513 by preventing the air outside the body from entering the suction channel 5512 between the inner surface of the body tube of the outer sheath 55 and the outer surface of the endoscope 51, ensuring the adsorption and fixation effect between the lumen wall of the human body and the body tube of the outer sheath 55.

In some exemplary embodiments, the second seal 555 may be a conical silicone rubber seal and the first seal 556 may be a silicone rubber seal. It should be understood that the first seal 556 and the second seal 555 may also be of other materials.

In some exemplary embodiments, as shown in FIG. 13I, the body tube is of a multi-layer structure and includes a spiral band layer 5515, a mesh tube layer 5516, an outer polymer layer 5517, and an inner polymer layer 5518. In an exemplary embodiment, the spiral band layer 5515 is disposed between the outer polymer layer 5517 and the inner polymer layer 5518, and the mesh tube layer 5516 is disposed between the outer polymer layer 5517 and the spiral band layer 5515, the outer polymer layer 5517 may overlay the mesh tube layer 5516 and the mesh tube layer 5516 may overlay the inner polymer layer 5518.

The multilayer structure of the body tube of the outer sheath 55 may include an inner polymer layer 5518, a spiral band layer 5515, a mesh tube layer 5516 and an outer polymer layer 5517 arranged sequentially from the inside to the outside. The outer polymer layer 5517 may be made of TPU (thermoplastic polyurethane elastomer) material or other material, and the surface of the outer polymer layer 5517 may or may not be provided with a coating, and if provided with a coating, it may be, for example, a hydrophilic coating, a Teflon coating or other coating. The inner polymer layer 5518 may be made of TPU (thermoplastic polyurethane elastomer) material or other material, and the surface of the inner polymer layer 5518 may or may not be provided with a coating, and if provided with a coating, it may be, for example, a hydrophilic coating, a Teflon coating or other coating, the mesh tube layer 5516 may be made of stainless steel or other material, and the spiral band layer 5515 may be made of stainless steel or other material. By this arrangement, the body tube of the outer sheath 55 is provided with a certain structural strength in order to transmit force to the endoscope 51 through the outer sheath 55, and the body tube of the outer sheath 55 can be bent and deformed for delivery in the human body.

The outer sheath 55 according to an embodiment of the present application is equipped with an expandable member 554 for anchoring and fixing, and can be fixed by suction through the suction channel 5512; its body tube is of a multilayer structure reinforced by a spiral band layer 5515 and a mesh tube layer 5516 such that the body tube is flexible but can provide a 1:1 torque transmission from the outer sheath 55 to the endoscope 51; the tube-locking mechanism 552 can realize the locking fit/unlocking fit between the body tube and the endoscope 51; delivery of the endoscope 51 in the human body can be achieved by movement of the outer sheath 55 and the endoscope 51 together and separate movement of the endoscope 51; the second seal 555 can block the body fluid in the human body from entering the suction channel 5512 between the outer sheath 55 and the endoscope 51, and the conical part 5551 of the second seal 555 also prevents the lumen wall, intestinal wall or mucosal folds of the human body from being caught by the outer sheath 55 during the advancement of the endoscope 51 and the outer sheath 55 in the human body; the first seal 556 can prevent body fluid in the human body from leaking out of the human body, and the negative pressure around the suction opening 5513 can be maintained by preventing air outside the human body from entering the suction channel 5512 between the outer sheath 55 and the endoscope 51.

During use, the expandable member 554 may be first inflated and simultaneously a suction force is applied by a suction apparatus to secure the outer sheath 55 to the intestinal or luminal wall of the human body; then, the tube-locking mechanism 552 can be released so that the body tube of the outer sheath 55 and the endoscope 51 are disengaged from being in locking fit; subsequently, the body tube of the outer sheath 55 can be held, and then the endoscope 51 can be pushed to move, so that the endoscope 51 can be avoided from being looped.

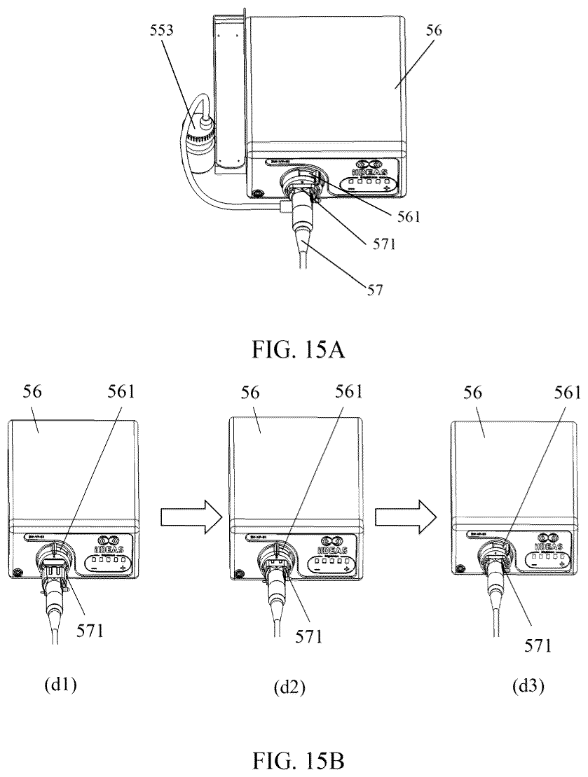

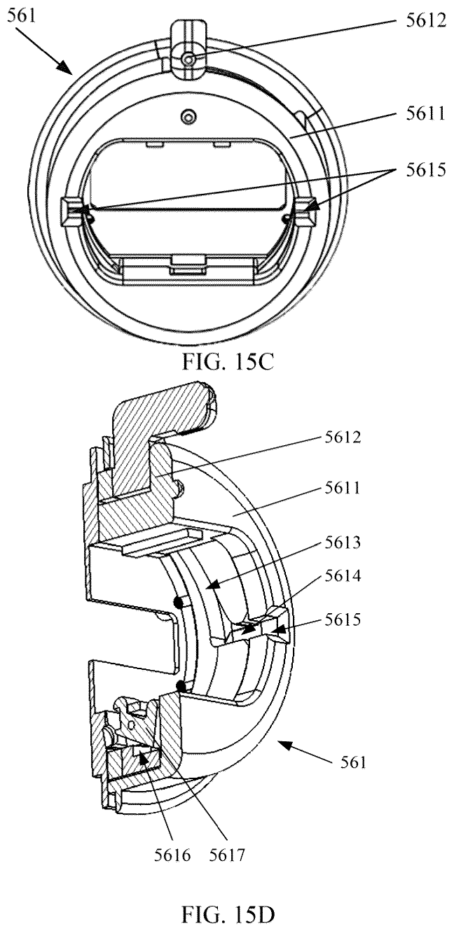

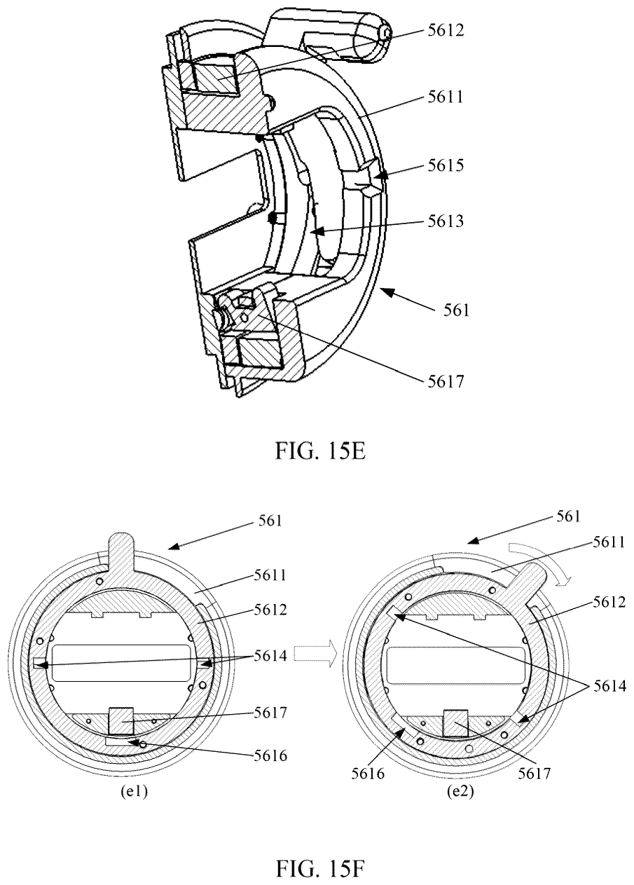

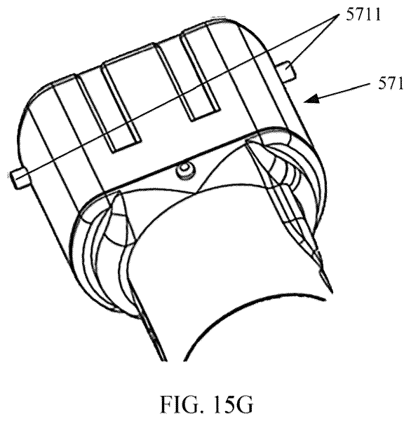

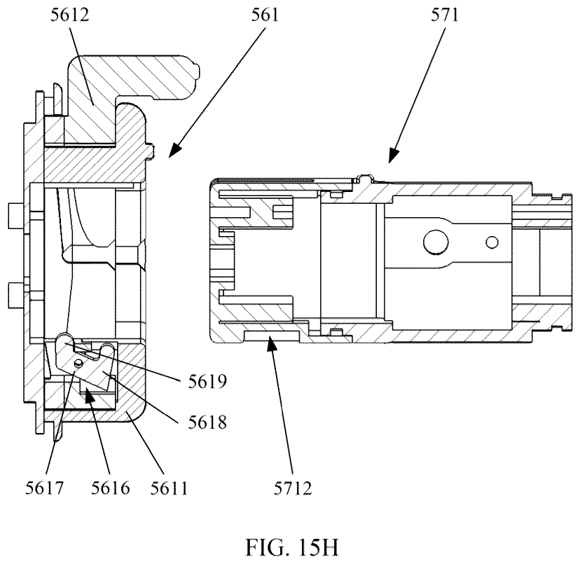

In some exemplary embodiments, the endoscope assembly 500 further includes an image capturing assembly disposed at the second end of the endoscope 51, and the endoscope magnetic anastomosis system further includes at least one video processor console. The video processor console includes a video processor 56, as shown in FIGS. 15A and 15B. The image capturing assembly is electrically connected to the video processor 56 by a cable 57 so as to transmit information in the human body captured by the image capturing assembly to the video processor 56 for processing and display with the video processor 56.

The video processor 56 has a main connector socket 561, and one end of the cable 57 electrically connected to the video processor 56 is provided with a main connector 571 for plug-in connection with the main connector socket 561. A locking structure is provided between the main connector socket 561 and the main connector 571 to lock the main connector socket 561 and the main connector 571 in plug-in connection. In an exemplary embodiment, the main connector 571 may be in a form of a plug.

A locking structure is provided between the main connector 571 of the cable 57 and the main connector socket 561 of the video processor 56, which can lock the main connector socket 561 and the main connector 571 in plug-in connection to prevent the main connector 571 from being separated from the main connector socket 561 during the delivery of the endoscope 51 in the human body.

In some exemplary embodiments, as shown in FIGS. 15C-15I, the main connector socket 561 includes a base 5611 and a rotatable locking ring 5612, the locking ring 5612 is rotatably mounted to the base 5611 to enable the main connector socket 561 to be switched between an initial state (shown in (e1) in FIGS. 15D and 15F) and a locked state (shown in (e2) in FIGS. 15E and 15F). The main connector socket 561 can be switched between the initial state and the locked state by rotation of the locking ring 5612.

In some exemplary embodiments, the locking structure provided between the main connector 571 and the main connector socket 561 comprises a positioning key 5711 that is provided on one of the locking ring 5612 and the main connector 571, and a locking slide slot 5613 that is provided in the other of the locking ring 5612 and the main connector 571 and extends in the circumferential direction. One end of the locking slide slot 5613 is an insertion end 5614. The positioning key 5711 is configured to be inserted into the locking slide slot 5613 from the insertion end 5614 when the main connector 571 is in plug-in connection with the main connector socket 561 in the initial state, and is configured to slide relative to the locking slide slot 5613 during the rotation of the locking ring 5612, so that the positioning key 5711 and the insertion end 5614 of the locking slide slot 5613 are misaligned and thus the main connector socket 561 switches to the locking state.

In some exemplary embodiments, as shown in FIGS. 15C to 15G, one or more positioning keys 5711 may be provided. For example, as shown in FIG. 15G, two positioning keys 5711 may be provided. The two positioning keys 5711 may be respectively provided on two sides of the main connector 571. Correspondingly, one or more locking slide slots 5613 may be provided to cooperate with one or more positioning keys 5711 in a one-to-one manner. For example, two locking slide slots 5613 may be provided. These two s locking slide slots 5613 may be provided on the locking ring 5612, may extend along the circumferential direction of the locking ring 5612, and may respectively cooperate with the two positioning keys 5711. When the main connector 571 is in plug-in connection with the main connector socket 561 in the initial state, the positioning keys 5711 can be inserted into the locking slide slot 5613 from the insertion end 5614 of the locking slide slot 5613; then, by rotating the locking ring 5612, the positioning keys 5711 can slide in the locking slide slot 5613, and then the positioning keys 5711 and the insertion end 5614 of the locking slide slot 5613 are misaligned, so as to prevent the positioning keys 5711 from coming out of the insertion end 5614 of the locking slide slot 5613, and further prevent the main connector 571 from being separated from the main connector socket 561. At this time, the main connector socket 561 is switched to the locked state, and the lock fixation of the main connector 571 and thus the main connector socket 561 is realized.

It should be understood that the positioning key 5711 may be provided on the locking ring 5612, and the locking slide slot 5613 may be provided on the main connector 571 and extend along the circumferential direction of the main connector 571, and the lock fixation of the main connector 571 and the main connector socket 561 can also be achieved. It should also be understood that the main connector 571 may also be provided to include a base 5611 and a locking ring 5612, with a positioning key 5711 provided in one of the locking ring 5612 and the main connector socket 561, and a circumferentially extending locking slide slot 5613 provided in the other of the locking ring 5612 and the main connector socket 561.

In some exemplary embodiments, as shown in FIGS. 15D and 15E, the locking slide slot 5613 is a spiral slot, and the positioning key 5711 is configured to, during rotation of the locking ring 5612, be pressed by the spiral groove to drive the main connector 571 to be further deeply inserted into the interior of the main connector socket 561.

The locking slide slot 5613 is a spiral slot, in the direction away from the insertion end 5614 of the locking slide slot 5613, the locking slide slot 5613 is screwed toward the side of the main connector socket 561 away from the main connector 571 so that during the rotation of the locking ring 5612, the groove wall of the locking slide slot 5613 and the positioning key 5711 can be pressed against each other, and under the action of the pressing force, the main connector 571 may be moved toward a side of the main connector socket 561 away from the main connector 571 (as shown in FIG. 15I, the main connector 571 is moved to the left from the position indicated in state (f1) to the position indicated in state (f2) by a distance of d), so that the main connector 571 more tightly plug into the main connector socket 561 to ensure a good connection between the main connector 571 and the main connector socket 561 and to prevent accidental pull-out of the main connector 571.

In some exemplary embodiments, where the positioning key 5711 is provided on the main connector 571 and the locking slide slot 5613 is provided in the locking ring 5612, as shown in FIGS. 15C to 15E, the base 5611 is provided with an insertion slot 5615, the insertion slot 5615 is configured to be in communication with the insertion end 5614 of the locking slide slot 5613 when the main connector socket 561 is in the initial state (as shown in FIG. 15D) and disconnect from the insertion end 5614 of the locking slide slot 5613 when the locking ring 5612 is rotated to switch the main connector socket 561 to the locked state (as shown in FIG. 15E).

The locking ring 5612 is provided with a locking slide slot 5613, and the base 5611 is provided with an insertion slot 5615, and when the main connector socket 561 is in the initial state, the insertion slot 5615 can be aligned with and in communication with the insertion end 5614 of the locking slide slot 5613, so that the positioning key 5711 passes through the insertion slot 5615 and the insertion end 5614 of the locking slide slot 5613 sequentially and then enters the locking slide slot 5613; during the rotation of the locking ring 5612, the insertion slot 5615 is misaligned and disconnected from the insertion end 5614 of the locking slide slot 5613 to prevent the positioning key 5711 in the locking slide slot 5613 from coming out of the insertion slot 5615.

In some exemplary embodiments, as shown in FIGS. 15D-15F, 15H, and 15I, the base 5611 is provided with a rotatable latch 5617, the locking ring 5612 is provided with a limit slot, and the latch 5617 is configured such that when the main connector socket 561 is in the initial state, the first portion 5618 of the latch 5617 is located within the limit slot and the second portion 5619 of the latch 5617 is located outside the limit slot, as shown in FIGS. 15D and 15H, to circumferentially secure the locking ring 5612 to the base 5611 and to maintain the main connector socket 561 in the initial state. The latch 5617 is further configured such that when the main connector 571 is in plug-in connection with the main connector socket 561 in the initial state, the second portion 5619 of the latch 5617 located outside the limit slot can rotate under the urging of the main connector 571 as shown in the state (f1) in FIG. 15I, thereby causing the latch 5617 to rotate out of the limit slot and enabling the locking ring 5612 to rotate.

The base 5611 is provided with a latch 5617, and the locking ring 5612 is provided with a rotatable limit slot, and the locking ring 5612 can be rotated or fixed relative to the base 5611 by cooperation between the latch 5617 and the limit slot. Specifically, when the main connector socket 561 is in the initial state, as shown in FIGS. 15D and 15H, the first portion 5618 of the latch 5617 is located in the limit slot, and the second portion 5619 of the latch 5617 protrudes from the limit slot. By cooperation between the latch 5617 and the limit slot, the locking ring 5612 and the base 5611 can be fixed in the circumferential direction to prevent the locking ring 5612 from rotating, so that the main connector socket 561 can be maintained in the initial state, and the plug-in connection between the main connector 571 and the main connector socket 561 can be facilitated. When the main connector 571 is in plug-in connection with the main connector socket 561 in the initial state, the second portion 5619 of the latch 5617 protruding from the limit slot can be rotated under the urging action of the main connector 571 as shown in the state (f1) in FIG. 15I, so that the latch 5617 entirely comes out of the limit slot. At this time, the locking ring 5612 can be rotated relative to the base 5611, so that the base 5611 switches to the locked state and locks the main connector 571 (as shown in the state (f2) in FIG. 15I), preventing the main connector 571 from accidentally separating from the main connector socket 561.

In some exemplary embodiments, as shown in FIGS. 15H and 15I, the main connector 571 is provided with an avoidance groove 5712, the avoidance groove 5712 is configured to, during plug-in connection between the main connector 571 and the main connector socket 561, receive the first portion 5618 of the latch 5617 disengaged from the limit slot (as shown in FIG. 15I), and further configured to drive the latch 5617 to rotate when the main connector 571 is separated from the main connector socket 561 to reset the latch 5617 so that the first portion 5618 is positioned within the limit slot and the second portion 5619 is positioned outside the limit slot, to circumferentially secure the locking ring 5612 to the base 5611.

The main connector 571 is provided with an avoidance groove 5712, and when the main connector 571 is in plug-in connection with the main connector socket 561 in the initial state, the latch 5617 as a whole is gradually rotated to come out of the limit slot (switching from the state shown in FIG. 15H to the state (f1) shown in FIG. 15I), and the avoidance groove 5712 can accommodate a portion of the latch 5617 disengaged (protruded) from the limit slot so as not to hinder the rotation of the latch 5617. In the process of gradually separating the main connector 571 and the main connector socket 561 in the plug-in state, the groove wall of the avoidance groove 5712 can push the latch 5617 and drive the latch 5617 to rotate in the opposite direction, so that the latch 5617 is gradually reset to a state in which a part of the latch is located in the limit slot of the base 5611 and the other part of the latch protrudes from the limit slot (as shown in FIG. 15H). At this time, the latch 5617 cooperates with the limit slot to limit and secure the locking ring 5612 and the base 5611 in the circumferential direction, and the main connector socket 561 is in the initial state as a whole.

In some exemplary embodiments, as shown in FIGS. 15H and 15I, the center of rotation of the latch 5617 is located in the middle of the latch 5617 and offset from the center of gravity of the latch 5617 so that the latch 5617 can maintain the first portion 5618 within the limit slot and the second portion 5619 outside the limit slot under its own weight (as shown in FIG. 15H).

The center of rotation of the latch 5617 may be located in the middle of the latch 5617 and offset from the center of gravity of the latch 5617, so that the latch 5617 can automatically reset and maintain the first portion 5618 in the limit slot and the second portion 5619 outside the limit slot under the action of its own weight, so that the latch 5617 can automatically fix the locking ring 5612 and the base 5611 in the circumferential direction after the main connector 571 is separated from the main connector socket 561.

When the main connector 571 and the main connector socket 561 according to an embodiment of the present application are connected, the main connector 571 can be inserted into the main connector socket 561 first (the main connector 571 can be inserted in the direction indicated by the straight arrow in the state (d1) in FIG. 15B), and before insertion, the positioning key 5711 of the main connector 571 can be aligned with the insertion slot 5615 on the base 5611 of the main connector socket 561, and as the main connector 571 is gradually inserted into the main connector socket 561, the positioning key 5711 is gradually inserted into the locking slide slot 5613 of the locking ring 5612; then, the locking ring 5612 of the main connector socket 561 is toggled or rotated downward (the locking ring 5612 can be toggled or rotated in the direction indicated by the curved arrow in the state (d2) in FIG. 15B) to slide the positioning key 5711 in the locking slide slot 5613 to lock the main connector 571 and the main connector socket 561, and the locking slide slot 5613 presses the positioning key 5711 inward to move the positioning key 5711 and the main connector 571 toward the side of the main connector socket 561 away from the main connector 571 to achieve a tight connection between the main connector 571 and the main connector socket 561 (as shown in state (d3) in FIG. 15B) to ensure a good video signal connection between the video processor 56 and the cable 57, and to prevent the main plug from being accidentally pulled out. Finally, the video processor 56 may be ready for operation.

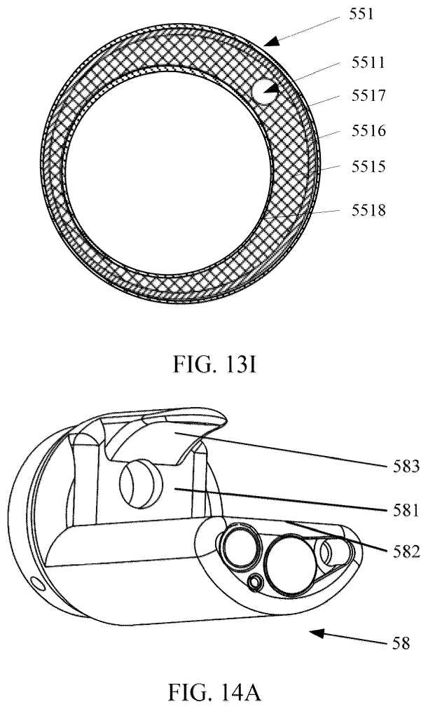

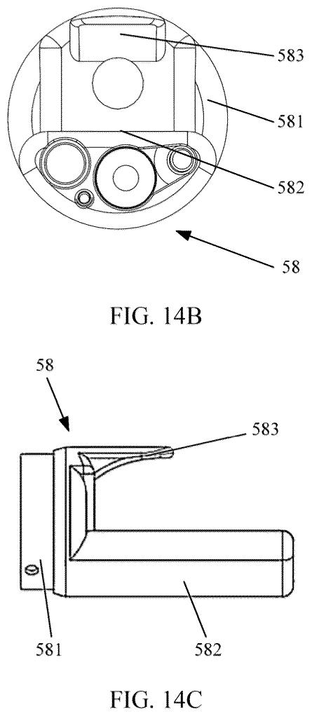

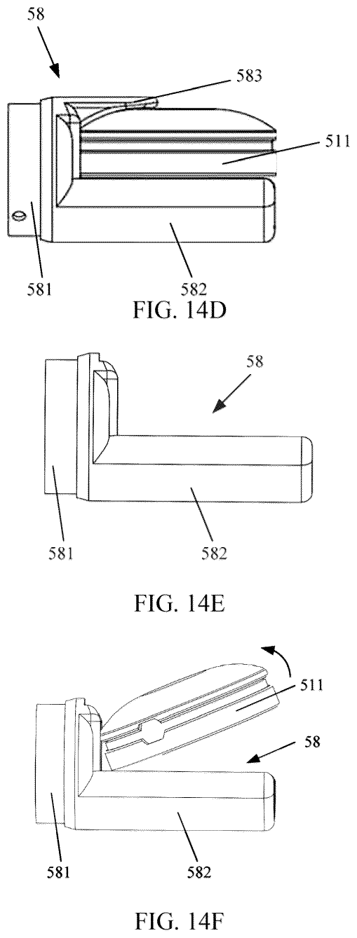

In some exemplary embodiments, the endoscope 51 includes a head assembly 58 disposed at a second end of the endoscope 51. As shown in FIGS. 14A to 14D, the head assembly 58 includes a head assembly body 581, one end surface of the head assembly body 581 is provided with a support part 582 and a limit part 583 which are disposed opposite to each other, for clamping the magnetic implant assembly 511 between the limit part 583 and the support part 582. The bottom of the magnetic implant assembly 511 is supported on the support part 582, and the top of the magnetic implant assembly 511 abuts against on the limit part 583. In an exemplary embodiment, the limit part 583 can be designed as a tongue-like structure (as shown in FIGS. 14A to 14D).

As shown in FIGS. 14E and 14F, a head assembly 58 is provided at the second end of the endoscope 51 (for example, the distal end into the human body), and a support part 582 is provided on one end surface of the head assembly body 581 of the head assembly 58, and the end surface of the head assembly body 581 and the support part 582 can cooperate to form a mounting base for the magnetic implant assembly 511, and the magnetic implant assembly 511 can be supported by the support part 582 and abut against the end surface of the head assembly body 581. As a result of this installation, the magnetic implant assembly 511 may be tilted (as shown in the direction of the arrow in FIG. 14F) by impacting the luminal wall, intestinal wall, or mucosal folds in the human body.

Therefore, in the embodiment of the present application, as shown in FIGS. 14A to 14D, a limit part 583 is further provided on the end surface of the head assembly body 581, and the latch 5617 and the support part 582 are oppositely disposed along the height direction of the magnetic implant assembly 511, so that the limit part 583 can contact the end surface of the magnetic implant assembly 511 on a side away from the support part 582, and restrain and limit the magnetic implant assembly 511 to prevent the magnetic implant assembly 511 from tilting when it impacts the lumen wall, intestinal wall or mucosal fold in the human body.

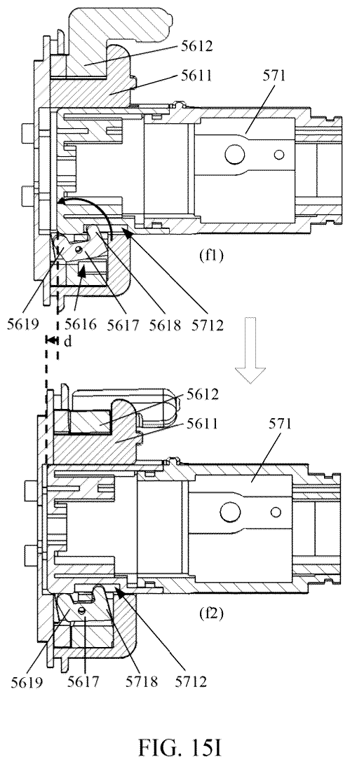

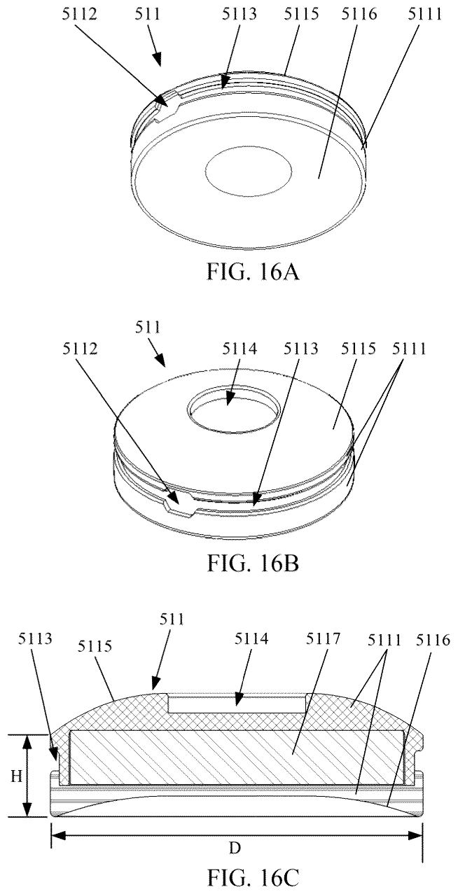

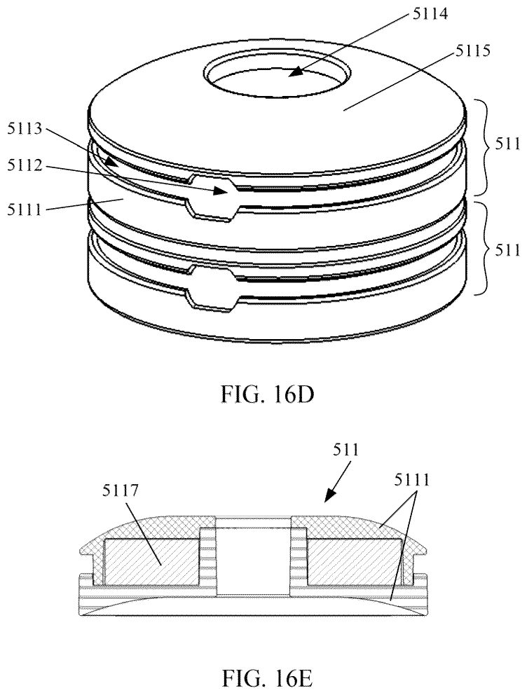

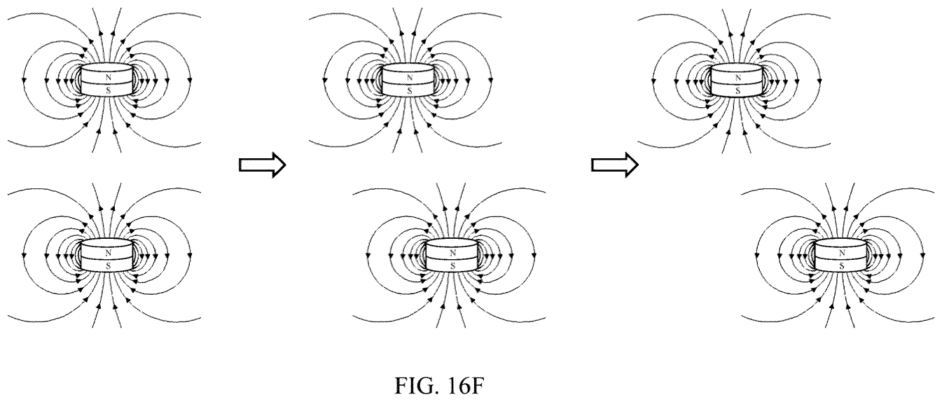

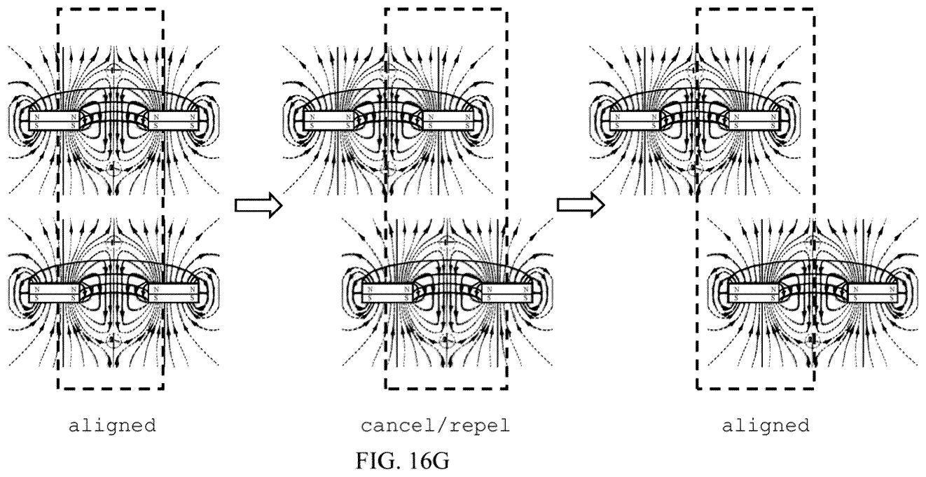

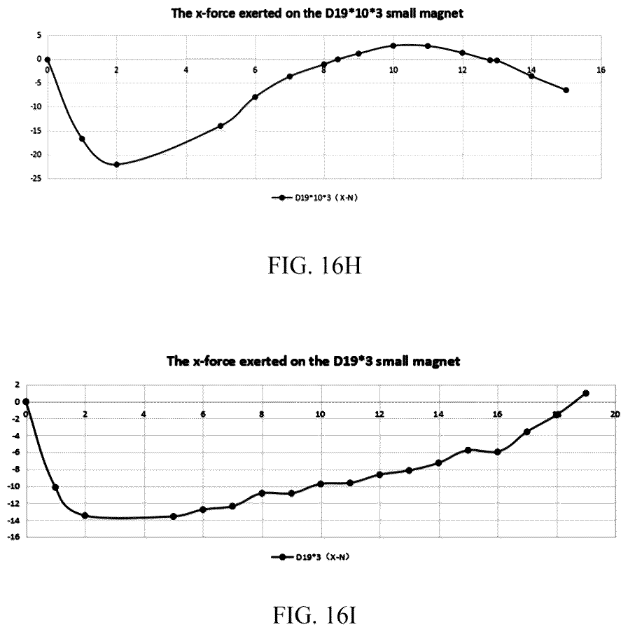

In some exemplary embodiments, as shown in FIGS. 16A to 16C, the magnetic implant assembly 511 of the endoscope 51 includes an enclosure 5111 and a magnet 5117, the magnet 5117 is disposed in the enclosure 5111, the enclosure 5111 includes a first end surface 5115 and a second end surface 5116 which are disposed opposite to each other, and the first end surface 5115 and the second end surface 5116 are both in an arc shape. In an exemplary embodiment, as shown in FIG. 14D, the second end surface 5116 of the enclosure 5111 may be supported on the support part 582 of the head assembly body 581, the first end surface 5115 of the enclosure 5111 may be in contact with a limit part 583 provided on the head assembly body 581, and a portion of the limit part 583 that is in contact with the first end surface 5115 of the enclosure 5111 is a curved surface, and the curved surface is adapted to the shape of the first end surface 5115 so as to prevent the magnetic implant assembly 511 from tilting by the limit part 583.

In some exemplary embodiments, as shown in FIGS. 16A-16C, the circumferential side surface of the enclosure 5111 of the magnetic implant assembly 511 is provided with a fixing ring groove 5113, and the snare wire 522 of the snare assembly 52 may be received within the fixing ring groove 5113 so that the snare wire 522 secures the magnetic implant assembly 511 tightly. The circumferential side surface of the enclosure 5111 of the magnetic implant assembly 511 is further provided with a recess 5112 for housing the tip of the snare catheter 521 of the snare assembly 52.

In some exemplary embodiments, as shown in FIGS. 16A-16C, the radii of curvature of the arc-shaped first end surface 5115 and the arc-shaped second end surface 5116 of the enclosure 5111 are different. For example, the radius of curvature of the first end surface 5115 is smaller than the radius of curvature of the second end surface 5116, and a groove 5114 is provided at the center of the first end surface 5115.

As shown in FIG. 16D, when the magnetic implant assemblies 511 of the two endoscopes 51 enter the human body and are magnetically coupled, the first end surface 5115 of the magnetic implant assembly 511 of the one endoscope 51 and the second end surface 5116 of the magnetic implant assembly 511 of the other endoscope 51 are opposite to each other and clamp the lumen wall or intestinal wall of the human body. Since the radius of curvature of the first end surface 5115 of the enclosure 5111 of the magnetic implant assembly 511 is less than the radius of curvature of the second end surface 5116, the distance s1 between a portion on the first end surface 5115 of the magnetic implant assembly 511 of one endoscope 51 surrounding the groove 5114 and the second end surface 5116 of the magnetic implant assembly 511 of another endoscope 51 is less than the distance s2 between an edge portion on the first end surface 5115 of the magnetic implant assembly 511 of the endoscope 51 away from the groove 5114 and the second end surface 5116 of the magnetic implant assembly 511 of another endoscope 51 Furthermore, the force F1 exerted by the portion of the first end surface 5115 of the magnetic implant assembly 511 of the endoscope 51 surrounding the groove 5114 on the lumen wall or the intestinal wall of the human body is greater than the force F2 exerted by the edge portion of the first end surface 5115 of the magnetic implant assembly 511 of the endoscope 51 away from the groove 5114 on the lumen wall or the intestinal wall of the human body. The adjacent application of different forces F1, F2 may improve the anastomosis formed by the magnetic implant assemblies 511 of the two endoscopes 51 and/or the healing of necrosis of the luminal or intestinal wall of the human body (and/or enable better controlled healing of the luminal or intestinal wall of the human body).

In some exemplary embodiments, as shown in FIG. 16C, the magnet 5117 of the magnetic implant assembly 511 is solid disc-shaped and is a permanent magnet.

In some exemplary embodiments, as shown in FIG. 10 and FIG. 16D, the at least one endoscopic assembly of the endoscopic magnetic anastomosis system comprises two endoscopic assemblies. The magnetic implant assemblies of the two endoscopic assemblies are respectively a first magnetic assembly configured to be located within a first luminal tissue region and a second magnetic implant assembly configured to be located within a second luminal tissue region (as shown in FIG. 10). The structures of the first magnetic implant assembly and the second magnetic implant assembly may be set to be the same. For example, the structures of each of the first magnetic implant assembly and the second magnetic implant assembly can be as shown in FIGS. 16A-16C.

An enclosure (i.e., a first enclosure) of the first magnetic implant assembly is provided with a fixing ring groove (i.e., a first fixing ring groove) on a circumferential side surface thereof to receive the snare wire of the snare assembly of one of the two endoscopic assemblies. An enclosure (i.e., a second enclosure) of the second magnetic implant assembly is provided with a fixing ring groove (i.e., a second fixing ring groove) on a circumferential side surface thereof to receive the snare wire of the snare assembly of the other of the two endoscopic assemblies.

The first enclosure of the first magnetic implant assembly has a first engagement surface, and the second enclosure of the second magnetic implant assembly has a second engagement surface. The first engagement surface and the second engagement surface are configured, when the first magnetic implant assembly and the second magnetic implant assembly are magnetically anastomosed with the first and second luminal issue regions interposed therebetween (as shown in FIG. 10), to face each other and be capable of exerting a non-uniform compressive force on the first luminal tissue region and the second luminal tissue region, enabling better controlled healing of the luminal or intestinal wall of the human body.

The first engagement surface is a concave curved surface with a first radius of curvature, and the second engagement surface is a convex curved surface with a second radius of curvature. The radius of curvature of the convex surface is smaller than that of the concave surface. The second end surface of the first enclosure of the first magnetic implant assembly can form the first engagement surface, and the first end surface of the second enclosure of the second magnetic implant assembly can form the second engagement surface.

The convex curved surface and the concave curved surface are configured such that when the first magnetic implant assembly and the second magnetic implant assembly are magnetically anastomosed, the convex curved surface protrudes towards the concave curved surface, and the axial distance between the convex curved surface and the concave curved surface along the central line direction of the first magnetic implant assembly increases as the radial distance from the central line of the first magnetic implant assembly increases, so as to exert the non-uniform compressive force on the first luminal tissue region and the second luminal tissue region.

In some exemplary embodiments, a magnet (i.e., a first magnet) disposed within the first enclosure of the first magnetic implant assembly and a magnet (i.e., a second magnet) disposed within the second enclosure of the second magnetic implant assembly are both solid disc-shaped permanent magnet. Thereby, when the first magnetic implant assembly and the second magnetic implant assembly are magnetically anastomosed, the center line of the first magnetic implant assembly and the center line of the second magnetic implant assembly can be automatically adjusted to an aligned state even when there is a certain deviation between them, with the magnetic poles of the ends of the first magnet and the second magnet adjacent to each other being opposite.

FIG. 16F is a schematic diagram of magnetic field interaction between two magnetic implant assemblies 511 (whose magnets 5117 are in a shape of a solid disc as shown in FIG. 16C) according to an embodiment of the present application, FIG. 16G is a schematic diagram of magnetic field interaction between two magnetic implant assemblies 511 (whose magnets 5117 are in a shape of a hollow ring as shown in FIG. 16E) in some cases, FIG. 16H is a schematic diagram of a relationship between the magnetic field force and distance between two magnetic implant assemblies 511 (whose magnets 5117 are in the shape of a solid disc as shown in FIG. 16C) according to an embodiment of the present application (where the horizontal axis represents the distance between the two magnetic implant assemblies, and the vertical axis represents the transverse magnetic field force between the two magnetic implant assemblies). FIG. 16I is a schematic diagram of a relationship between the magnetic field force and distance between two magnetic implant assemblies 511 (whose magnets 5117 are in a shape of a hollow ring as shown in FIG. 16E) (where the horizontal axis represents the distance between the two magnetic implant assemblies, and the vertical axis represents the transverse magnetic field force between the two magnetic implant assemblies) in some cases. According to FIGS. 16F to 16I, compared to setting the magnet 5117 of the magnetic implant assembly 511 in the hollow annular shape shown in FIG. 16E, the magnet 5117 of the magnetic implant assembly 511 is disposed in the solid disc shape shown in FIG. 16C so that the central axes of two such magnetic implant assemblies 511 can automatically adjust to an aligned state even when they deviate by a certain distance, allowing the magnetic implant assembly 511 to have unique self-alignment performance, which can better improve the alignment performance of the magnetic implant assemblies 511 of the two endoscopes 51 so that the magnetic implant assemblies 511 of the two endoscopes 51 can be better aligned to the formed anastomosis.

In some exemplary embodiments, as shown in FIG. 16C, the outer diameter D of the magnetic implant assembly 511 is 8 mm-30 mm, such as 15 mm-23 mm; the height H of the outer surface of the magnetic implant assembly 511 is 4 mm-8 mm.

In some exemplary embodiments, the width of the magnetic implant assembly 511 is larger than the width of the snare channel of the endoscope 51. For example, the outer diameter D of the magnetic implant assembly 511 (i.e., the width of the magnetic implant assembly 511), in the embodiment shown in FIG. 16C, is larger than the diameter of the snare channel (i.e., the width of the snare channel, not shown), preventing the magnetic implant assembly 511 from entering or passing through the snare channel.

In some exemplary embodiments, the magnet 5117 of the magnetic implant assembly 511 may be a strong neodymium magnet, and the enclosure 5111 of the magnetic implant assembly 511 may be a biocompatible enclosure 5111 (e.g., polycarbonate or other material).

In some exemplary embodiments, the endoscopic magnetic anastomosis system further includes a magnet detector 600 and the magnet detector 600 is configured to detect the position of the magnetic implant assembly 511 of the endoscope 51 within the human body.

The magnet detector 600 can detect the position of the magnetic implant assembly 511 of the endoscope 51 in the human body, so that the medical worker can know whether the magnetic implant assembly 511 is in place, and when the magnetic implant assembly 511 is not in place, the magnetic implant assembly 511 can be conveniently located and moved to the appropriate position.

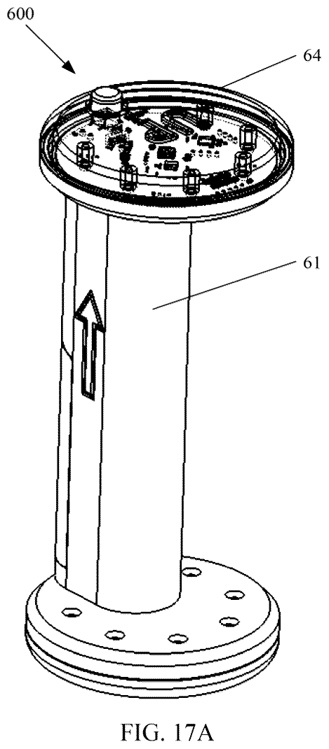

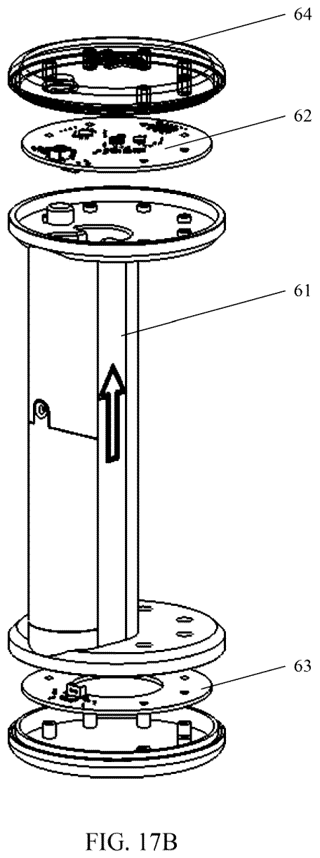

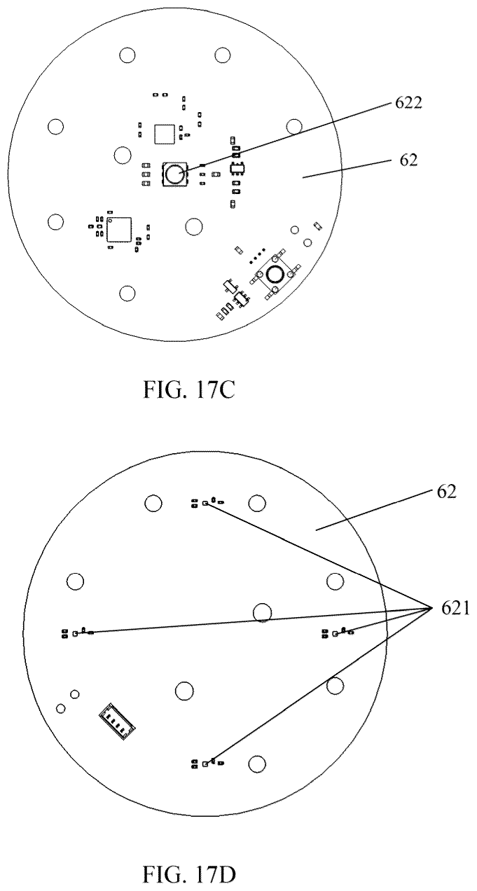

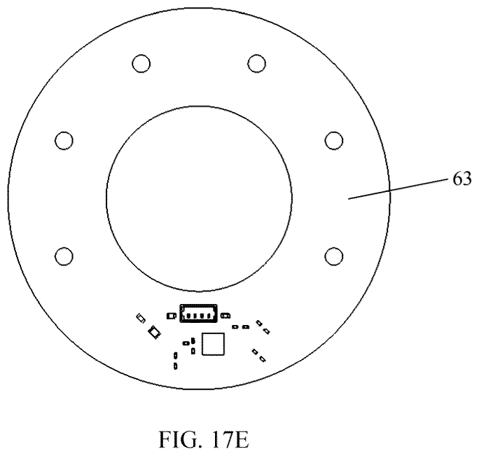

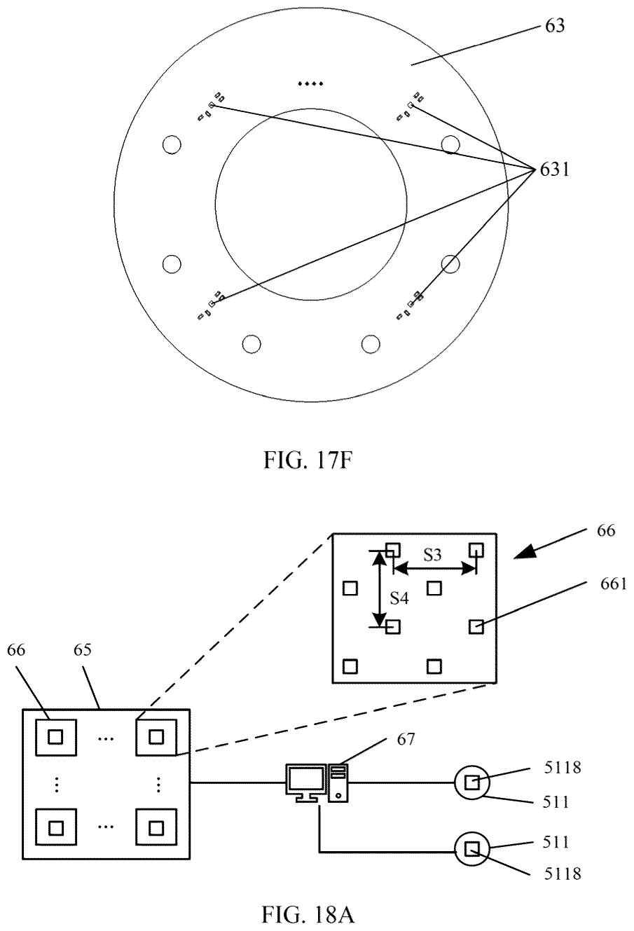

In some exemplary embodiments, as shown in FIGS. 17A-17F, the magnet detector 600 includes a detector body 61, a first printed circuit board 62, a second printed circuit board 63, and a processing unit. The first printed circuit board 62 is provided at the first end of the detector body 61, and a first group of magnetometers 621 is provided thereon. The second printed circuit board 63 is provided at a second end of the detector main body 61 opposite to the first end of the detector main body 61, and a second group of magnetometers 631 is provided thereon. The processing unit is arranged to be able to receive measurement data from the first group of magnetometers 621 and the second group of magnetometers 631, and to determine the position of the magnetic implant assembly 511 of the endoscope 51 in the human body from the data.

The magnet detector 600 may be a portable magnet detector 600, and the detector body 61 thereof may be provided with a grip part for taking and carrying the magnet detector; the first printed circuit board 62 and the second printed circuit board 63 are respectively provided at two ends (e.g., upper and lower ends) of the detector body 61, and the first group of magnetometers 621 and the second group of magnetometers 631 are respectively provided on the first printed circuit board 62 and the second printed circuit board 63, the first group of magnetometers 621 and the second group of magnetometers 631 can be used to measure an external magnetic field (e.g., the strength and direction of the magnetic field can be measured). The processing unit can be mounted to the detector body 61 and receive measurement data from the first group of magnetometers 621 and the second group of magnetometers 631, in order to determine the position of the magnetic implant assembly 511 of the endoscope 51 in the human body based on this data.

In some exemplary embodiments, as shown in FIG. 17D, the first group of magnetometers 621 includes four magnetometers distributed in a square array, and the center of the square array coincides with the center of the first printed circuit board 62. As shown in FIG. 17F, the second group of magnetometers 631 includes four magnetometers distributed in a square array, and the center of the square array coincides with the center of the second printed circuit board 63. In an exemplary embodiment, the spacing between the first printed circuit board 62 and the second printed circuit board 63 may be 15 cm, the four magnetometers in the first group of magnetometers 621 may be 2.5 cm apart from each other in both transverse and longitudinal directions, and the four magnetometers in the second group of magnetometers 631 may be 2.5 cm apart from each other in both transverse and longitudinal directions.

In the magnet detector 600 according to an embodiment of the present application, the first printed circuit board (upper layer PCB) 62 and the second printed circuit board (lower layer PCB) 63, spaced 15 cm vertically, may be horizontally arranged and parallel to each other. The first printed circuit board 62 has a first group of magnetometers 621 comprising four magnetometers that are positioned symmetrically in a square formation, with equal spacing of 2.5 cm along both width and length of the square. The second printed circuit board 63 has a second group of magnetometers 631 comprising four magnetometers that are positioned symmetrically in a square formation, with equal spacing of 2.5 cm along both width and length of the square.

The magnet detector 600 includes multiple layers of magnetometers arranged in a grid pattern (e.g., two layers of magnetometers: a first group of magnetometers 621 and a second group of magnetometers 631 disposed on the first printed circuit board 62 and the second printed circuit board 63, respectively), allowing for long range spatial magnetic field measurements. The magnet detector 600 can be used to detect the position of the magnet 5117 on a side of the second printed circuit board 63 away from the first printed circuit board 62, for example, it can be used to detect the magnet 5117 below it, for example, the magnet detector 600 can provide accurate sensing of the magnet 5117 within a range of 3 cm to 15 cm beneath the second printed circuit board 63. When in use, the magnet detector 600 can be moved over a human body (e. g., the abdomen of the human body), to detect the position of the magnetic implant assembly 511 in the human body.

The magnet detector 600 has a calibration function to eliminate background noise. The magnet detector 600 undergoes an initial zero-point calibration, setting all magnetometers to a baseline value of zero. The magnetic field above the first printed circuit board 62 acts as a background noise reference, and the first group of magnetometers 621 can continuously measure magnetic field disturbances from external sources; the background noise reading is then subtracted from a lower magnetic field measurement (detected by the second group of magnetometers 631) including both the background noise and any target magnetic signal to obtain the target magnetic signal.

The magnet detector 600 can perform signal preprocessing for long-distance detection, and the first group of magnetometers 621 and the second group of magnetometers 631 can perform noise reduction using a high-resolution magnetic field measurement magnetometer MMC56x3.

When processing the measurement data of the first group of magnetometers 621 and the second group of magnetometers 631, a weighted average method may be used. The signals from the first group of magnetometers 621 and the second group of magnetometers 631 are combined using a weighted average, where the more reliable or noise-free measurements contribute more significantly to the final result.

The processing unit may use artificial intelligence (AI)-powered signals so as to: enable accurate detection even when the signal strength is low (e.g., in the range of 10 cm-15 cm below the second printed circuit board 63); and learn to differentiate between real magnetic signals and interference signals using spatial data across multiple magnetometers.

The processing unit may integrate an AI-neural network processing function.

Inputs to the neural network structure: The neural network structure receives preprocessed magnetic field data (x, y, z axes) from all the first group of magnetometers 621 and the second group of magnetometers 631 as a multi-dimensional feature vector. Each of the first group of magnetometers 621 and the second group of magnetometers 631 contributes to the perception of the magnetic environment by the neural network structure.

Output of the neural network structure: The neural network structure uses a sigmoid activation function in the output layer to produce a probability between 0 and 1. This represents the likelihood of a magnet target being present.

The execution pipeline of the neural network processing is: reading the data of the first group of magnetometers 621 and the second group of magnetometers 631→preprocessing→running deep neural network (DNN) inference→outputting the detection result on LED.

Edge AI optimization capabilities for neural network processing: the model is quantized (INT 8) and stripped of unnecessary complexity using TensorFlow Lite for microcontrollers, allowing it to fit within the MCU's limited memory and computation budget.

In some exemplary embodiments, the magnet detector 600 further includes an indicator 622, and the indicator 622 is electrically connected to the processing unit and configured to indicate whether the magnetic implant assembly 511 of the endoscope 51 has been detected. In an exemplary embodiment, as shown in FIGS. 17B and 17C, the indicator 622 is an LED mounted on the first printed circuit board 62, and the first end of the detector body 61 is further provided with a transparent cover 64 that covers the first printed circuit board 62.

The detection status of the magnet detector 600 is visually indicated by the LED on the first printed circuit board 62 at the top, which indicates whether the magnetic implant assembly 511 in the human body has been detected by whether the LED is on. For example, the LED is lit to indicate that the magnetic implant assembly 511 in the human body is detected, and the LED is not lit to indicate that the magnetic implant assembly 511 in the human body is not detected.

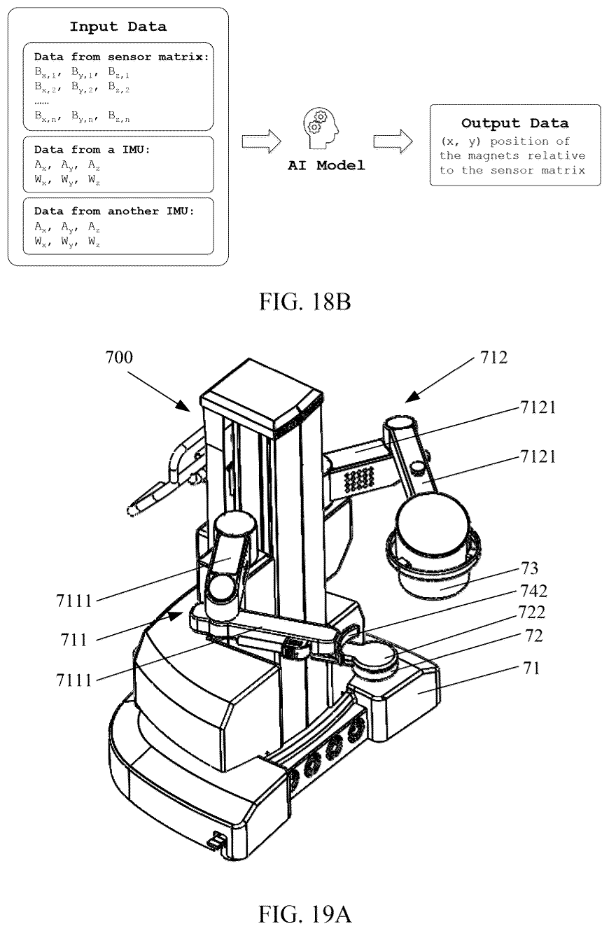

In other exemplary embodiments, as shown in FIGS. 18A and 18B, the magnet detector 600 includes a mounting substrate 65, at least one sensor module 66, and a processing module 67. Each sensor module 66 may include eight magnetic sensors 661. The eight magnetic sensors 661 are regularly arranged in four rows and each row has two magnetic sensors 661. The two magnetic sensors 661 are a first magnetic sensor 661 and a second magnetic sensor 661, respectively, and the two magnetic sensors 661 in each row are in a misaligned arrangement with the two magnetic sensors 661 in an adjacent row. The inertial measurement unit (IMU) 5118 may be embedded in the magnetic implant assembly 511 of the endoscope 51, and the processing module 67 is arranged to receive data from the sensor module 66 and data of the IMU 5118 embedded in the magnetic implant assembly 511, and determine the position of the magnetic implant assembly 511 of the endoscope 51 in the human body based on the data.

In an exemplary embodiment, the number of sensor modules 66 is between 1-32. In an exemplary embodiment, the spacing S3 between two magnetic sensors 661 located in a same row is 5 cm, a transverse misalignment spacing between the first magnetic sensor 661 in each row and the first magnetic sensor 661 in the adjacent row is 2.5 cm, a transverse misalignment spacing between the second magnetic sensor 661 in each row and the second magnetic sensor 661 in the adjacent row is 2.5 cm, and the longitudinal spacing between the magnetic sensors 661 in any two adjacent rows is 2.5 cm, so that the spacing S4 between the two rows of magnetic sensors 661 spaced by one row is 5 cm.

The magnet detector 600 has a modular sensor matrix including N (e.g., 1-32) sensor module(s) 66 with a dimension of 10 cm×10 cm, each sensor module houses eight magnetic sensors 661, the magnetic sensors 661 are arranged in a 4×4 checkerboard pattern and positioned at alternating nodes of the grid. Each magnetic sensor 661 outputs a 3D magnetic field vector (Bₓ, B_{y}, B_{z}). It in total generates an 8N×3 data matrices per sampling interval.

The IMU 5118 may be embedded in the magnetic implant assembly 511 of the endoscope 51. In an exemplary embodiment having two endoscopes 51, each of the magnetic implant assemblies 511 of two endoscopes 51 is embedded with an IMU 5118. Each IMU 5118 can measure 3D acceleration (Aₓ, A_{y}, A_{z}) and 3D angular velocity (Wₓ, W_{y}, W_{z}).

The processing module 67 may host a trained AI model to process the sensor matrix data and the input data of the IMU 5118, and may outputs real-time (x, y) coordinates of the magnetic implant assemblies 511 relative to the sensor matrix.

The magnet detector 600 according to an embodiment of the present application is a modular real-time magnetic positioning system with a configurable sensor module 66 and AI-driven tracking. This system introduces a novel magnetic localization architecture combining a modular sensor matrix, a magnetic implant assembly 511 (the magnet 5117 of which is a permanent magnet) equipped with an IMU 5118, and an AI processing unit (processing module 67) to achieve real-time positional tracking. When in use, the magnet detector 600 may be positioned underneath or on the back side of the human body to detect the position of the magnetic implant assembly 511 in the human body above or in front thereof.

The configurable sensor matrix consists of a user-deployable array of 1-32 sensor modules 66, and each sensor module 66 is embedded with 8 magnetic sensors 661 to measure the 3D magnetic field, thus enabling adaptable spatial coverage.

The AI processing unit enables sensor fusion: 3D magnetic field data (from the sensor matrix) and 6-degree-of-freedom inertial data (from the IMU 5118) can be integrated to enhance positional and dynamic tracking.

AI model of AI processing unit: the deep learning model is trained to predict (x, y) positions of a pair of magnetic implant assemblies 511 of the two endoscope assemblies 500 relative to the sensor matrix in real time, leveraging both magnetic and inertial inputs.

According to the AI model architecture shown in FIG. 18B, it can be seen that:

The input data of the AI processing unit includes:
1) Data from the sensor matrix: (B_{x,1}, B_{y,1}, B_{z,1}), (B_{x,2}, B_{y,2}, B_{z,2}), . . . (B_{x,n}, B_{y,n}, B_{z,n});
2) Data from one IMU 5118: (Aₓ, A_{y}, A_{z}), (Wₓ, W_{y}, W_{z}); and
3) Data from another IMU 5118: (Aₓ, A_{y}, A_{z}), (Wₓ, W_{y}, W_{z}).

The output data of the AI processing unit includes the (x, y) position of the magnetic implant assembly 511 relative to the sensor matrix.

In some exemplary embodiments, the endoscope magnetic anastomosis system further includes a magnetic navigation console 700, and the magnetic navigation console 700 is configured to drive the magnetic implant assembly 511 of the endoscope 51 to move to a set position in the human body by the action of a magnetic field.

When the endoscope 51 is delivered in the human body, if the position of the magnetic implant assembly 511 does not reach the appropriate position (set position), the magnetic navigation console 700 can be used to drive the magnetic implant assembly 511 to move in the human body so that the magnetic implant assembly 511 reaches the appropriate position.

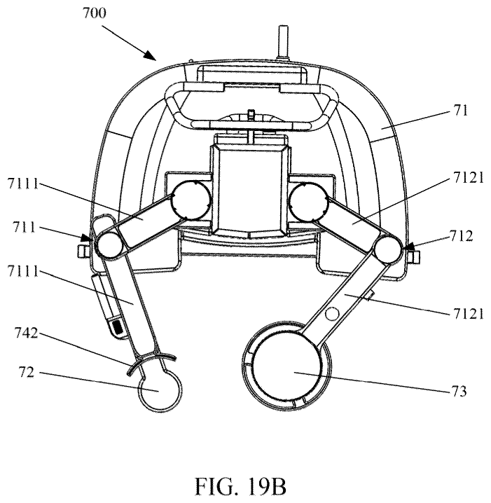

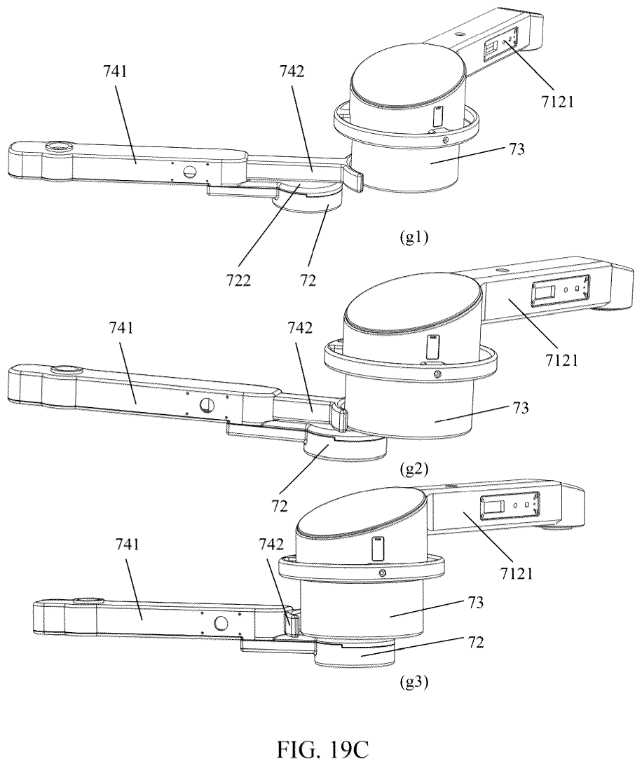

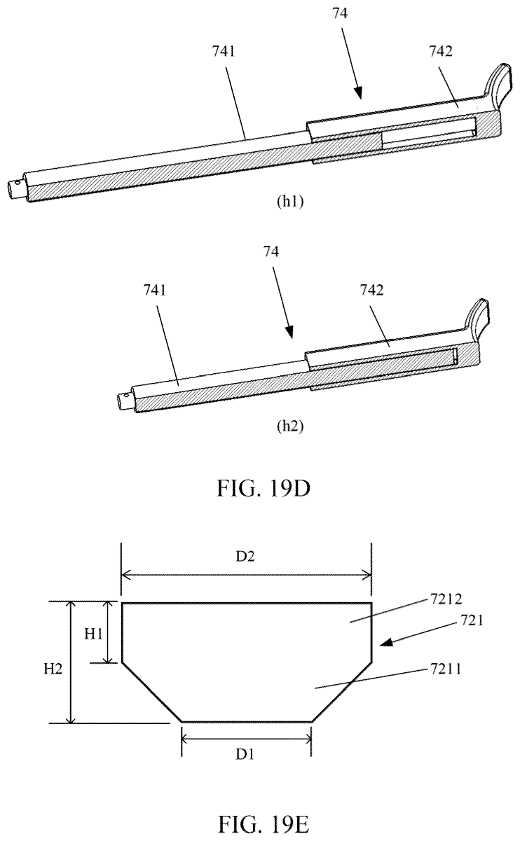

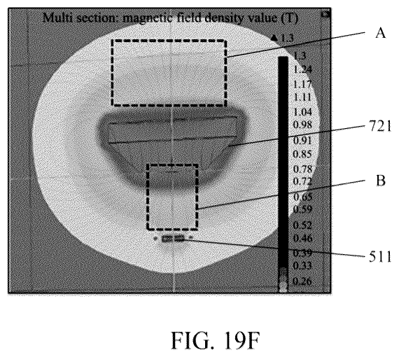

In some exemplary embodiments, as shown in FIGS. 19A-19B, the magnetic navigation console 700 includes a mounting bracket 71, a first magnetic actuator 72, and a second magnetic actuator 73.

The mounting bracket 71 includes a movable first mounting arm 711 and a movable second mounting arm 712. The first magnetic actuator 72 is mounted to the first mounting arm 711, and is arranged to drive the magnetic implant assembly 511 of one endoscope assembly 500 of the two endoscope assemblies 500 to move in the human body by the action of a magnetic field. The second magnetic actuator 73 is mounted to the second mounting arm 712, and is arranged to drive the magnetic implant assembly 511 of another endoscope assembly 500 of the two endoscope assemblies 500 to move in the human body by the action of a magnetic field. The first magnetic actuator 72 and the second magnetic actuator 73 are disposed to be movable to a state in which they overlap in the vertical direction (as shown in the state (g3) in FIG. 19C) so as to move the magnetic implant assemblies 511 of the two endoscopes 51 to a state where they overlap in the vertical direction in the human body (as shown in FIG. 16D). In an exemplary embodiment, the second magnetic actuator 73 is disposed such that the magnetic field strength generated by the second magnetic actuator is greater than the magnetic field strength generated by the first magnetic actuator 72, and the first magnetic actuator 72 is located below the second magnetic actuator 73 when the first magnetic actuator 72 and the second magnetic actuator 73 overlap in the vertical direction.

The magnetic navigation console 700 is a dual magnetic actuator system having a first magnetic actuator 72 and a second magnetic actuator 73, the first magnetic actuator 72 and the second magnetic actuator 73 are supported by a first mounting arm 711 and a second mounting arm 712, respectively, and the first mounting arm 711 and the second mounting arm 712 are movable so that the first magnetic actuator 72 and the second magnetic actuator 73 are movable, thereby driving the magnetic implant assemblies 511 of the two endoscopes 51 to move within the human body. The first magnetic actuator 72 and the second magnetic actuator 73 can move to a state where they vertically overlap, which may remotely bring the two magnetic implant assemblies 511 within the human body close enough for mating.

Therefore, when the first magnetic actuator 72 and the second magnetic actuator 73 overlap in the vertical direction, the first magnetic actuator 72 is located below the second magnetic actuator 73, so that the first magnetic actuator 72 is closer to the magnetic implant assembly 511 driven by the first magnetic actuator, and the second magnetic actuator 73 is away from the magnetic implant assembly 511 driven by the first magnetic actuator. However, since the magnetic field strength generated by the second magnetic actuator 73 is greater than that generated by the first magnetic actuator 72, the first magnetic actuator 72 can still drive the corresponding magnetic implant assembly 511 to move by action of magnetic field, and the second magnetic actuator 73 can still drive the corresponding magnetic implant assembly 511 to move by action of magnetic field.

The second magnetic actuator 73 can be sized larger than the first magnetic actuator 72 such that the magnetic field strength generated by the second magnetic actuator 73 is greater than the magnetic field strength generated by the first magnetic actuator 72. Of course, the magnetic field strength generated by the second magnetic actuator 73 may be made greater than the magnetic field strength generated by the first magnetic actuator 72 not only by the size, but also by material selection, structural setting, and the like.

In some exemplary embodiments, as shown in FIG. 19E, the first magnetic actuator 72 includes a permanent magnet 721, and the permanent magnet 721 of the first magnetic actuator 72 includes a conical section 7211 having a lower end cross-sectional dimension smaller than an upper end cross-sectional dimension.

The permanent magnet 721 of the first magnetic actuator 72 includes a conical section 7211, and the provision of the conical section 7211 can be used to establish a magnetic flux concentration (as shown in the rectangular frame B in FIG. 19F), which helps the first magnetic actuator 72 to drive the corresponding magnetic implant assembly 511 to move within the human body, thereby facilitating the two magnetic implant assemblies 511 in the human body to be sufficiently close to each other to cooperate.

In some exemplary embodiments, as shown in FIG. 19E, the permanent magnet 721 of the first magnetic actuator 72 further includes a cylindrical section 7212 extends upward from the upper end of the conical section 7211, and the cross-sectional dimension of the cylindrical section 7212 is equal to the cross-sectional dimension of the upper end of the conical section 7211. The permanent magnet 721 of the first magnetic actuator 72 further includes a cylindrical section 7212 extends upward from the upper end of the conical section 7211, and the arrangement of the cylindrical section 7212 enables the first magnetic actuator 72 to have a larger dimension as a whole, thereby enabling the first magnetic actuator 72 to generate a greater magnetic field strength. As can be seen from FIG. 19F, compared with the magnetic field generated by the cylindrical section 7212 (as shown in the rectangular frame A in FIG. 19F), the magnetic field generated by the conical section 7211 can achieve magnetic flux concentration (as shown in the rectangular frame B in FIG. 19F).

In some exemplary embodiments, as shown in FIG. 19E, the diameter D1 of the bottom surface of the conical section 7211 is 20 mm-60 mm, the diameter of the upper end of conical section 7211 is equal to the diameter D2 of cylindrical section 7212 and ranges from 60 mm-100 mm, and the sum H2 of the height of the conical section 7211 and the height H1 of the cylindrical section 7212 is 10 mm-50 mm. For example, in one example, the diameter D1 of the bottom surface of the conical section 7211 is 40 mm, the diameter of the upper end surface of the conical section 7211 and the diameter D2 of the cylindrical section 7212 are both 80 mm, the sum H2 of the heights of the conical section 7211 and the cylindrical section 7212 is 30 mm, the height of the conical section 7211 may be 20 mm, and the height H1 of the cylindrical section 7212 may be 10 mm.

It should be understood that the size of the permanent magnet 721 of the first magnetic actuator 72 is not limited to the above range, and may be adjusted according to actual needs.

In some exemplary embodiments, the second magnetic actuator 73 includes a permanent magnet; alternatively, the second magnetic actuator 73 includes an electromagnetic coil; alternatively, the second magnetic actuator 73 includes a permanent magnet and an electromagnetic coil disposed above or below the permanent magnet.

The second magnetic actuator 73 may drive the magnetic implant assembly 511 to move within the human body by a magnetic field generated by a permanent magnet and/or an electromagnetic coil.

In some exemplary embodiments, as shown in FIGS. 19A to 19B, the first mounting arm 711 is longitudinally slidably mounted to the mounting bracket 71 and hinged with the mounting bracket 71, and the first mounting arm 711 includes a plurality of connecting arms 7111 hinged sequentially; the second mounting arm 712 is longitudinally slidably mounted to the mounting bracket 71 and hinged with the mounting bracket 71. The second mounting arm 712 includes a plurality of connecting arms 7121 hinged sequentially.

The first mounting arm 711 and the second mounting arm 712 are slidably mounted to the mounting bracket 71 in the longitudinal direction (vertical direction) so that the first magnetic actuator 72 and the second magnetic actuator 73 mounted to the first mounting arm 711 and the second mounting arm 712 can move up and down in the vertical direction; the first mounting arm 711 includes a plurality of connecting arms 7111 hinged sequentially, the plurality of connecting arms 7111 of the first mounting arm 711 are rotatable along a longitudinal axis such that the first magnetic actuator 72 mounted to the first mounting arm 711 is movable in a horizontal plane; the second mounting arm 712 includes a plurality of connecting arms 7121 hinged sequentially, the plurality of connecting arms 7121 of the second mounting arm 712 are rotatable along a longitudinal axis such that the second magnetic actuator 73 mounted to the second mounting arm 712 is movable in a horizontal plane.

The structure of the plurality of connecting arms 7121 of the first mounting arm 711 and the plurality of connecting arms 7121 of the second mounting arm 712 is configured such that the first magnetic actuator 72 and the second magnetic actuator 73 are movable in a three-dimensional space so that the first magnetic actuator 72 and the second magnetic actuator 73 move to a vertically overlapping state.

In some exemplary embodiments, since the first magnetic actuator 72 is located below the second magnetic actuator 73 when the first magnetic actuator 72 and the second magnetic actuator 73 overlap in the vertical direction, the anti-friction coating 722 (as shown in FIG. 19C) may be provided at the upper end of the first magnetic actuator 72, and/or the anti-friction coating may be provided at the lower end of the second magnetic actuator 73.

By providing the anti-friction coating 722 at the upper end of the first magnetic actuator 72 and/or providing the anti-friction coating at the lower end of the second magnetic actuator 73, the friction force between the first magnetic actuator 72 and the second magnetic actuator 73 can be reduced during the movement of the first magnetic actuator 72 and the second magnetic actuator 73 to a vertically overlapping state or during the separation from the vertically overlapping state.

In some exemplary embodiments, the thickness of the anti-friction coating 722 at the upper end of the first magnetic actuator 72 may be 5 mm-20 mm, and the material of the anti-friction coating may be Teflon (polytetrafluoroethylene)/acetal; and/or the thickness of the anti-friction coating at the lower end of the second magnetic actuator 73 may be 5 mm to 20 mm, and the material of the anti-friction coating may be Teflon (polytetrafluoroethylene)/acetal.

It should be understood that the thickness and material of the anti-friction coating 722 at the upper end of the first magnetic actuator 72 and the thickness and material of the anti-friction coating 722 at the lower end of the second magnetic actuator 73 are not limited to the above ranges, and may be adjusted according to actual needs.

In some exemplary embodiments, as shown in FIGS. 19A-19D, the magnetic navigation console 700 further includes a push rod mechanism 74, the push rod mechanism 74 is configured to extend and retract and capable of pushing one of the first magnetic actuator 72 and the second magnetic actuator 73 to move so that the first magnetic actuator 72 and the second magnetic actuator 73 are away from each other. In an exemplary embodiment, the push rod mechanism 74 may be mounted to the first mounting arm 711 and may urge the second magnetic actuator 73 to move. It should be understood that the push rod mechanism 74 may also be mounted to the second mounting arm 712 and may urge the first magnetic actuator 72 in motion.

During the movement of the first magnetic actuator 72 and the second magnetic actuator 73 to the vertically overlapping state (as moved from the state (g1) in FIG. 19C through the state (g2) and to the state (g3)), the push rod mechanism 74 may be contracted (as changed from the state (h1) to the state (h2) in FIG. 19D) so as not to hinder the movement of the first magnetic actuator 72 and the second magnetic actuator 73 to the vertically overlapping state; during separation of the first magnetic actuator 72 and the second magnetic actuator 73 from the vertically overlapping state (as moved from the state (g3) in FIG. 19C through the state (g2) and to the state (g1)), the push rod mechanism 74 may be extended (as changed from the state (h2) to the state (h1) in FIG. 19D) to push one of the first magnetic actuator 72 and the second magnetic actuator 73 so that the first magnetic actuator 72 and the second magnetic actuator 73 are away from each other.

In some exemplary embodiments, as shown in FIG. 19D, the push rod mechanism 74 includes a second linear actuator 741 and a push rod 742, the second linear actuator 741 is mounted to an end of the first mounting arm 711 close to the first magnetic actuator 72, and the push rod 742 is connected to a drive end of the second linear actuator 741 and configured to extend and retract under the driving of the second linear actuator 741.

The drive end of the second linear actuator 741 is linearly telescopically movable and can drive the push rod 742 to extend and retract so that during the movement of the first magnetic actuator 72 and the second magnetic actuator 73 to the vertically overlapping state, the drive end of the second linear actuator 741 can drive the push rod 742 to retract (as shown by the state (h2) in FIG. 19D) to avoid the second magnetic actuator 73; during the separation of the first magnetic actuator 72 and the second magnetic actuator 73 from the vertically overlapping state, the drive end of the second linear actuator 741 may drive the extension of the push rod 742 (as shown by state (h1) in FIG. 19D) to push one of the first magnetic actuator 72 and the second magnetic actuator 73 so that the first magnetic actuator 72 and the second magnetic actuator 73 are away from each other.

The magnetic navigation console 700 according to an embodiment of the present application is an innovative motorized dual magnetic actuator system for remotely navigating magnetic implant assemblies 511 (e.g., two magnetic implant assemblies 511) within a human body. The dual magnetic actuator system primarily includes a small auxiliary magnetic actuator (a first magnetic actuator 72) and a large main magnetic actuator (a second magnetic actuator 73) which are respectively supported by a first mounting arm 711 and a second mounting arm 712 (e.g., two dual planar articulating arms), allowing vertical overlapping of the first magnetic actuator 72 and the second magnetic actuator 73 which can remotely bring the two magnetic implant assemblies 511 close enough for mating. The dual magnetic actuator system also includes a motorized pusher (a push rod mechanism 74) for pushing apart the first magnetic actuator 72 and the second magnetic actuator 73. The permanent magnet 721 of the first magnetic actuator 72 includes a conical section 7211 for establishing a magnetic flux concentration to help to mate the two magnetic implant assemblies 511. The second magnetic actuator 73 may include a permanent magnet and an electromagnetic coil, and the electromagnetic force of the second magnetic actuator 73 may be output continuously and in pulse mode (100V, 60 A). The magnetic navigation console 700 can be designed in a compact single cart design for enhancing mobility and ease of use.

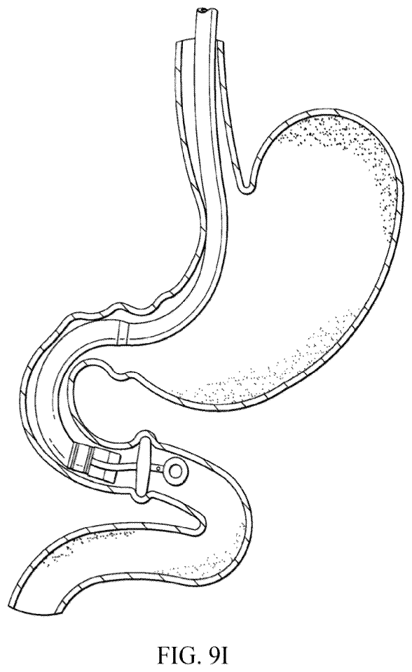

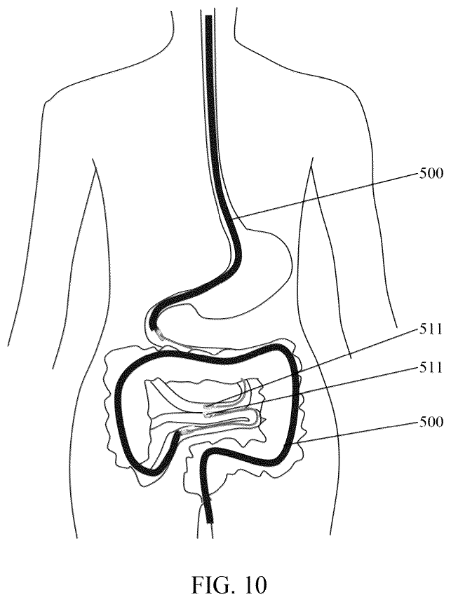

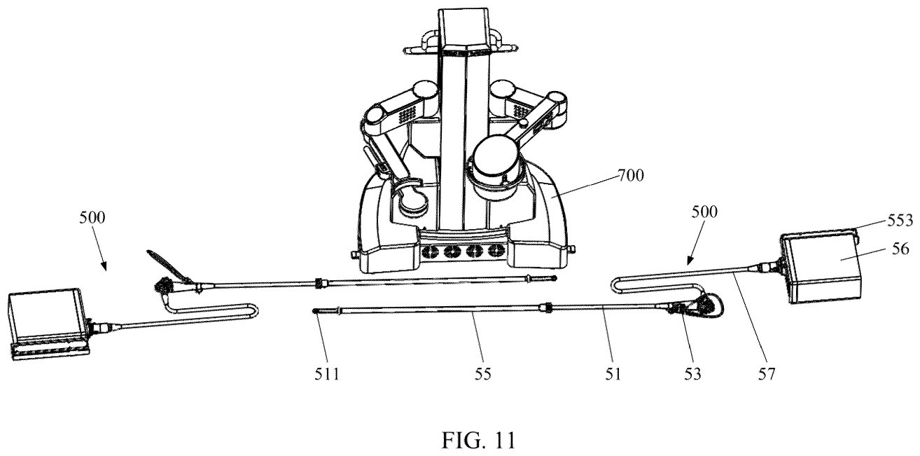

In summary, as shown in FIG. 11, the endoscopic magnetic anastomosis system according to an embodiment of the present application may include two endoscope assemblies 500, two video processors 56 and an external pressure source 553 used in conjunction with the two endoscope assemblies 500, and may also include a magnet detector 600 and a magnetic navigation console 700.

An embodiment of the present application also provides an adjustable snare mechanism, as shown in FIGS. 12A to 12D, including a snare assembly 52 and a snare guide assembly 53, wherein the snare assembly 52 includes a snare catheter 521 and a snare wire 522 passing through the snare catheter 521; the snare guide assembly 53 includes a base body 531, a first movable member 532 and a second movable member 533, the first movable member 532 and the second movable member 533 are both movably mounted on the base body 531, and the first movable member 532 is connected with the snare wire 522 and arranged to drive the snare wire 522 to move; the second movable member 533 is connected to the snare catheter 521 and is arranged to drive the snare catheter 521 to move; the snare wire 522 is disposed to be movable relative to the snare catheter 521 upon movement of at least one of the first movable member 532 and the second movable member 533 to cause the snare wire 522 to be retracted into the snare catheter 521 and extended out of the snare catheter 521.

The adjustable snare mechanism according to an embodiment of the present application may be the adjustable snare mechanism of the endoscopic magnetic anastomosis system described above, and may have some or all of the features of the adjustable snare mechanism described above.

An embodiment of the present application also provides an outer sheath 55, as shown in FIGS. 13A to 13I, including a body tube having a first end and an opposite second end, and a tube-locking mechanism 552 mounted at the first end of the body tube. The body tube has an endoscope channel 5519 extending from the first end of the body tube to the second end of the body tube for passage of the endoscope 51. The tube-locking mechanism 552 is configured to be capable of being in locking fit with the endoscope 51 passing through the endoscope channel 5519 to fix the endoscope 51 to the body tube, and to disengage from being in locking fit with the endoscope 51 passing through the endoscope channel 5519 to enable the endoscope 51 to slide and rotate relative to the body tube.

The outer sheath 55 according to an embodiment of the present application may be the outer sheath 55 of the endoscopic magnetic anastomosis system described above, and may have some or all of the characteristics of the outer sheath 55 described above.

An embodiment of the present application also provides a plug-in connection structure, as shown in FIGS. 15A to 15I, including a main connector socket 561 and a main connector 571, the main connector 571 is in plug-in connection with the main connector 571, and a locking structure is provided between the main connector socket 561 and the main connector 571 to lock the main connector socket 561 and the main connector 571 in plug-in connection.

The main connector socket 561 includes a base 5611 and a rotatable locking ring 5612, and the locking ring 5612 is rotatably mounted to the base 5611 to enable the main connector socket 561 to be switched between an initial state and a locked state.

One of the locking ring 5612 and the main connector 571 is provided with a positioning key 5711, the other of the locking ring 5612 and the main connector 571 is provided with a locking slide slot 5613 extending in the circumferential direction, one end of the locking slide slot 5613 is an insertion end 5614, and the positioning key 5711 is configured to be inserted into the locking slide slot 5613 from the insertion end 5614 when the main connector 571 is in plug-in connection with the main connector socket 561 in the initial state, and is configured to slide in the locking slide slot 5613 during the rotation of the locking ring 5612, so that the positioning key 5711 and the insertion end 5614 of the locking slide slot 5613 are misaligned and the main connector socket 561 switches to the locking state.

The plug-in connection structure according to an embodiment of the present application may be a plug-in connection structure between the video processor 56 and the cable 57 of the endoscopic magnetic anastomosis system described above, and may have some or all of the features of the outer sheath 55 described above. Of course, the plug-in connection structure can also be used for plug-in connection between the main connector 571 and the main connector socket 561 of other devices.

An embodiment of the present application also provides a magnet detector 600, as shown in FIGS. 17A to 17F, the magnet detector 600 is configured to detect the position of the magnet 5117, and the magnet detector 600 includes a detector body 61, a first printed circuit board 62, a second printed circuit board 63, and a processing unit. The first printed circuit board 62 is provided at the first end of the detector body 61, and a first group of magnetometers 621 is provided thereon. The second printed circuit board 63 is provided at a second end of the detector body 61 opposite to the first end, and a second group of magnetometers 631 is provided thereon. The processing unit is arranged to be able to receive measurement data from the first group of magnetometers 621 and the second group of magnetometers 631 and determine the position of the magnet 5117 based on the data.

The magnet detector 600 according to an embodiment of the present application may be the magnet detector 600 of the endoscopic magnetic anastomosis system described above, and may have some or all of the characteristics of the magnet detector 600 described above. Of course, the magnet detector 600 may also be used to drive the location of other magnets other than the magnetic implant assembly 511 of the endoscopic magnetic anastomosis system.

An embodiment of the present application also provides another magnet detector 600, as shown in FIGS. 18A and 18B, the magnet detector 600 is arranged to be able to detect the position of the magnet 5117, and the magnet detector 600 includes a mounting substrate 65, at least one sensor module 66, and a processing module 67. Each sensor module 66 includes eight magnetic sensors 661, the eight magnetic sensors 661 are regularly distributed in four rows with two magnetic sensors 661 per row. The two magnetic sensors 661 in each row are in a misaligned arrangement with the two magnetic sensors 661 in an adjacent row. The processing module 67 is arranged to receive data from the sensor module 66 and data of the IMU 5118 embedded in the magnet 5117, and determine the position of the magnet 5117 based on the data.

The magnet detector 600 according to an embodiment of the present application may be the magnet detector 600 of the endoscopic magnetic anastomosis system described above, and may have some or all of the characteristics of the magnet detector 600 described above. Of course, the magnet detector 600 may also be used to drive the location of other magnets other than the magnetic implant assembly 511 of the endoscopic magnetic anastomosis system.

An embodiment of the present application also provides a magnetic navigation console 700, as shown in FIGS. 19A to 19F, the magnetic navigation console 700 is arranged to drive the magnet 5117 to move by the action of a magnetic field, the magnetic navigation console 700 includes a mounting bracket 71, a first magnetic actuator 72, and a second magnetic actuator 73, and the mounting bracket 71 includes a first mounting arm 711 and a second mounting arm 712 which are movable; the first magnetic actuator 72 is mounted to the first mounting arm 711; the second magnetic actuator 73 is mounted to the second mounting arm 712, and the first magnetic actuator 72 and the second magnetic actuator 73 are disposed to be movable to a state where they overlap in the vertical direction.

The magnetic navigation console 700 according to an embodiment of the present application may be the magnetic navigation console 700 of the endoscopic magnetic anastomosis system described above, and may have some or all of the features of the magnetic navigation console 700 described above. Of course, the magnetic navigation console 700 may also be used to drive the movement of other magnets other than the magnetic implant assembly 511 of the endoscopic magnetic anastomosis system.

Various embodiments are described herein, but the description is exemplary and not restrictive, and it will be apparent to those of ordinary skill in the art that many more embodiments and implementations are possible within the scope of the embodiments described herein. Although many possible combinations of features are shown in the drawings and discussed in the detailed description, many other combinations of the disclosed features are also possible. Unless specifically limited, any feature or element of any embodiment may be used in combination with, or may be substituted for, any other feature or element of any other embodiment.

The present application includes and contemplates combinations with features and elements known to those of ordinary skill in the art. The embodiments, features, and elements already disclosed herein may also be combined with any conventional features or elements to form the unique inventive schemes defined by the claims. Any feature or element of any embodiment may also be combined with features or elements from other inventive schemes to form another unique inventive schemes defined by the claims. Accordingly, it should be understood that any of the features shown and/or discussed in this application may be implemented alone or in any suitable combination. Accordingly, the embodiments are not subject to any other restrictions other than those in accordance with the appended claims and their equivalent substitutions. Furthermore, various modifications and changes can be made within the scope of protection of the appended claims.

Furthermore, when describing representative embodiments, the specification may have presented a method and/or process as a particular sequence of steps. However, to the extent that the method or process does not depend on the specific order of steps described herein, the method or process should not be limited to the specific order of steps described. As will be understood by those of ordinary skills in the art, other order of steps is also possible. Accordingly, a particular order of steps set forth in the specification should not be construed as limitations on the claims. Furthermore, the claims for the method and/or process should not be limited to the steps which are performed in the written order. Those skilled in the art can readily understand that these orders can be changed and the changed orders still remain within the spirit and scope of the embodiments of the present application.

While various embodiments in accordance with the disclosed principles have been described above, it should be understood that they have been presented by way of example only, and are not limiting. Thus, the breadth and scope of the example embodiments described in the present disclosure should not be limited by any of the above-described exemplary embodiments, but should be defined only in accordance with the claims and their equivalents issuing from this disclosure. Furthermore, the above advantages and features are provided in described embodiments, but shall not limit the application of such issued claims to processes and structures accomplishing any or all of the above advantages.

For example, “assembly,” “device,” “portion,” “segment,” “member,” “body,” or other similar terms should generally be construed broadly to include one part or more than one part attached or connected together.

Various terms used herein have special meanings within the present technical field. Whether a particular term should be construed as such a “term of art” depends on the context in which that term is used. “Connected,” “connecting,” “attached,” “attaching,” “anchored,” “anchoring,” “in communication with,” “communicating with,” “associated with,” “associating with,” or other similar terms should generally be construed broadly to include situations where attachments, connections, and anchoring are direct between referenced elements or through one or more intermediaries between the referenced elements. These and other terms are to be construed in light of the context in which they are used in the present disclosure and as one of ordinary skill in the art would understand those terms in the disclosed context. The above definitions are not exclusive of other meanings that might be imparted to those terms based on the disclosed context.

As referred to in the present disclosure, a computing device, controller, manipulator, master input device, a processor, and/or a system may be a virtual machine, computer, node, instance, host, and/or device in a networked or non-networked computing environment. A networked computing environment may be a collection of devices connected by communication channels that facilitate communications between devices and allow devices to share resources. Also as referred to in the present disclosure, a computing device may be a device deployed to execute a program operating as a socket listener and may include software instances.

Resources may encompass any type of resource for running instances including hardware (such as servers, clients, mainframe computers, networks, network storage, data sources, memory, central processing unit time, scientific instruments, and other computing devices), as well as software, software licenses, available network services, and other non-hardware resources, or a combination thereof.

A networked computing environment may include, but is not limited to, computing grid systems, distributed computing environments, cloud computing environment, etc. Such networked computing environments include hardware and software infrastructures configured to form a virtual organization comprised of multiple resources that may be in geographically disperse locations.

Furthermore, the coverage of the present application and any patents issuing from the present application may extend to one or more communications protocols, including TCP/IP.

Words of comparison, measurement, and timing such as “at the time,” “equivalent,” “during,” “complete,” and the like should be understood to mean “substantially at the time,” “substantially equivalent,” “substantially during,” “substantially complete,” etc., where “substantially” means that such comparisons, measurements, and timings are practicable to accomplish the implicitly or expressly stated desired result.

Additionally, the section headings herein are provided for consistency with the suggestions under 37 C.F.R. 1.77 or otherwise to provide organizational cues. These headings shall not limit or characterize the invention(s) set out in any claims that may issue from this disclosure. Specifically, a description of a technology in the “Background” is not to be construed as an admission that technology is prior art to any invention(s) in this disclosure. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings herein.

1. An endoscopic magnetic anastomosis system, comprising at least one endoscopic assembly comprising:
an endoscope having a first end and an opposite second end, the endoscope comprising a snare channel extending from the first end to the second end and a magnetic implant assembly disposed at the second end; and
an adjustable snare mechanism, comprising a snare assembly passing through the snare channel, and comprising a snare catheter and a snare wire passing through the snare catheter for selectively tightening and releasing the magnetic implant assembly.

2. The endoscopic magnetic anastomosis system according to claim 1, wherein the adjustable snare mechanism further comprises a snare guide assembly that comprises a base body and a motion mechanism movably installed on the base body, and wherein the motion mechanism is connected to and is configured to drive at least one of the snare wire and the snare catheter, such that there is a relative movement between the snare wire and the snare catheter to allow the snare wire to selectively extend and retract relative to the snare catheter.

3. The endoscopic magnetic anastomosis system according to claim 2, wherein the motion mechanism comprises a first movable member and a second movable member, wherein the first movable member is connected to the snare wire and configured to be able to drive the snare wire to move and the second movable member is connected to the snare catheter and configured to be able to drive the snare catheter to move, such that there is the relative movement between the snare wire and the snare catheter when at least one of the first movable member and the second movable member moves.

4. The endoscopic magnetic anastomosis system according to claim 3, wherein the base body of the snare guide assembly is provided with a first slide rail and a second slide rail extending along a length direction of the base body, the first movable member is configured to be able to slide along the first slide rail, and the second movable member is configured to be able to slide along the second slide rail.

5. The endoscopic magnetic anastomosis system according to claim 3,
wherein the snare guide assembly further comprises a releasable locking mechanism mounted to the first movable member, the releasable locking mechanism is configured to be able to be in locking fit with the base body to secure the first movable member to the base body and to also be able to disengage from being in locking fit with the base body to enable the first movable member to move relative to the base body; and/or
wherein the snare guide assembly further comprises a unlockable locking device mounted to the second movable member, the unlockable locking device is configured to be able to be in locking fit with the base body to secure the second movable member to the base body, and also be able to disengage from being in locking fit with the base body to enable the second movable member to move relative to the base body; or wherein the second movable member is in friction fit with the base body such that the second movable member can remain fixed relative to the base body under the action of the frictional force between the second movable member and the base body, and also can overcome the friction force to move relative to the base body under action of an external force.

6. The endoscopic magnetic anastomosis system according to claim 5, wherein the releasable locking mechanism comprises a rotary locking mechanism comprising a knob and a clamping member, wherein the knob is connected to the clamping member, and wherein the knob is configured to be rotatable in two opposite directions to drive the clamping member to clamp and release the base body accordingly.

7. The endoscopic magnetic anastomosis system according to claim 1, wherein the endoscopic assembly further comprises:
an outer sheath comprising a body tube having a first end and an opposite second end and a tube-locking mechanism mounted at the first end of the body tube, the body tube further has an endoscopic channel extending from the first end of the body tube to the second end of the body tube for passage of the endoscope, the tube-locking mechanism is configured to be able to be in locking fit with the endoscope to secure the endoscope with the body tube and also be able to disengage from being in locking fit with the endoscope to enable the endoscope to slide and rotate relative to the body tube.

8. The endoscopic magnetic anastomosis system according to claim 7, wherein the tube-locking mechanism comprises:
a locking seat mounted at the first end of the body tube;
a rotatable member rotatably mounted to the locking seat and provided with a spiral driving part, wherein a spiral center line of the spiral driving part coincides with a rotation center line of the rotatable member; and
a plurality of locking blocks movably mounted to the locking seat, and configured to be in driving fit with the spiral driving part so as to be translatable in a radial direction of the spiral driving part as the spiral driving part rotates, end portions of the plurality of locking blocks adjacent to each other collectively defining an opening for the endoscope to pass through;
wherein the plurality of locking blocks are configured such that when the rotatable member rotates in a first direction, the plurality of locking blocks translate radially inwardly toward the spiral center line of the spiral driving part to narrow the opening to clamp the endoscope, and when the rotatable member rotates in a second direction opposite to the first direction, the plurality of locking blocks translate radially outwardly away from the spiral center line of the spiral driving part to enlarge the opening to release the endoscope.

9. The endoscopic magnetic anastomosis system according to claim 7, wherein the outer sheath further comprises:
a first seal disposed at the first end of the body tube to form a seal between the body tube and an outer surface of the endoscope; and
a second seal disposed at the second end of the body tube to form a seal between the body tube and the outer surface of the endoscope;
wherein a gap between the inner surface of the body tube and the outer surface of the endoscope forms a suction channel, the body tube has one or more suction openings provided at the second end of the body tube in a circumferential direction of the body tube, and a suction connector provided at the first end of the body tube, the suction openings and the suction connector are located between the first seal and the second seal, two ends of the suction channel are in communication with the suction openings and the suction connector, respectively, and the suction connector is configured to be connected with a suction apparatus for providing a negative pressure.

10. The endoscopic magnetic anastomosis system according to claim 9, wherein the second seal is disposed proximate the suction openings and the first seal is disposed proximate the suction connector; and
wherein the first seal has a first sealing rib in seal fit with the outer surface of the endoscope; one end of the second seal away from the first seal is provided with a conical part, a tapered end of the conical part away from the first seal being in seal fit with the outer surface of the endoscope; and the second seal is further provided with a second sealing rib closer to the first seal than the conical part, the second sealing rib being in seal fit with the outer surface of the endoscope.

11. The endoscopic magnetic anastomosis system according to claim 7, wherein the outer sheath further comprises:
an expandable member sleeved on an outer surface of the body tube at the second end of the body tube, the body tube is provided with a gas channel extending between an inner surface and the outer surface of the body tube in an axial direction of the body tube, the gas channel is in communication with the expandable member at the second end of the body tube, and is in communication with an external pressure source at the first end of the body tube, so that the expandable member expands outwardly in a radial direction of the body tube from a non-expanded state to an expanded state or retracts from the expanded state to the non-expanded state in response to action of the external pressure source.

12. The endoscopic magnetic anastomosis system according to claim 11, wherein the endoscopic assembly further comprises:
the external pressure source configured to be able to provide a fixed volume of gas to the expandable member to expand the expandable member outwardly in a radial direction of the body tube.

13. The endoscopic magnetic anastomosis system according to claim 12, wherein the external pressure source comprises:
a housing;
a first linear actuator mounted within the housing, the first linear actuator having a drive end configured to be movable between a first position and a second position;
a syringe mounted within the housing, the syringe comprising a syringe barrel and a syringe plunger movably mounted in the syringe barrel, the syringe barrel has an injection port in communication with the gas channel, the drive end of the first linear actuator is connected with the syringe plunger and configured to be able to drive the syringe plunger to reciprocate relative to the syringe barrel to respectively supply gas to or draw gas from the expandable member through the injection port; and
two position sensors mounted within the housing, configured to detect a position of the drive end of the first linear actuator.

14. The endoscopic magnetic anastomosis system according to claim 1, wherein the endoscope further comprises a head assembly disposed at the second end of the endoscope, the head assembly comprises a head assembly body, one end surface of the head assembly body is provided with a support part and a limit part which are disposed opposite to each other, for clamping the magnetic implant assembly between the limit part and the support part, a bottom portion of the magnetic implant assembly is supported on the support part, and a top portion of the magnetic implant assembly abuts against the limit part.

15. The endoscopic magnetic anastomosis system according to claim 1, wherein the endoscopic assembly further comprises an image capturing assembly disposed at the second end of the endoscope, wherein the endoscopic magnetic anastomosis system further comprises a video processor electrically connected to the image capturing assembly via a cable, and a main connector provided at one end of the cable for electrically connected to the video processor, and wherein the video processor has a main connector socket for plug-in connection with the main connector, and a locking structure is provided between the main connector socket and the main connector to lock the main connector socket and the main connector which are in plug-in connection.

16. The endoscopic magnetic anastomosis system according to claim 15, wherein the main connector socket comprises a base and a rotatable locking ring, the locking ring rotatably mounted to the base to enable the main connector socket to be switched between an initial state and a locked state; and
wherein the locking structure comprises a positioning key that is provided on one of the locking ring and the main connector, and a locking slide slot that is provided in the other of the locking ring and the main connector and extends in a circumferential direction, one end of the locking slide slot is an insertion end, and the positioning key is configured to be able to be inserted into the locking slide slot from the insertion end when the main connector is in plug-in connection with the main connector socket in the initial state, and is arranged to be able to slide relative to the locking slide slot during the rotation of the locking ring, so that the positioning key and the insertion end of the locking slide slot are misaligned and thus the main connector socket switches to the locking state.

17. The endoscopic magnetic anastomosis system according to claim 1, wherein the at least one endoscopic assembly comprises two endoscopic assemblies, the magnetic implant assemblies of the two endoscopic assemblies are respectively a first magnetic assembly configured to be located within a first luminal tissue region and a second magnetic implant assembly configured to be located within a second luminal tissue region;
wherein the first magnetic implant assembly comprises a first enclosure and a first magnet disposed within the first enclosure, the first enclosure being provided with a first fixing ring groove on a circumferential side surface of the first enclosure to receive the snare wire of the snare assembly of one of the two endoscopic assemblies, and the first enclosure having a first engagement surface;
wherein the second magnetic implant assembly comprises a second enclosure and a second magnet disposed within the second enclosure, the second enclosure being provided with a second fixing ring groove on a circumferential side surface of the second enclosure to receive the snare wire of the snare assembly of the other of the two endoscopic assemblies, and the second enclosure having a second engagement surface;
wherein the first engagement surface and the second engagement surface are configured, when the first magnetic implant assembly and the second magnetic implant assembly are magnetically anastomosed with the first and second luminal issue regions interposed therebetween, to face each other and be capable of exerting a non-uniform compressive force on the first luminal tissue region and the second luminal tissue region; and
wherein the first and second magnets are both solid disc-shaped permanent magnet such that when the first magnetic implant assembly and the second magnetic implant assembly are magnetically anastomosed, the center line of the first magnetic implant assembly and the center line of the second magnetic implant assembly can be automatically adjusted to an aligned state even when there is a certain deviation between them.

18. The endoscopic magnetic anastomosis system according to claim 17, wherein the first engagement surface is a concave curved surface with a first radius of curvature, and the second engagement surface is a convex curved surface with a second radius of curvature, the radius of curvature of the convex surface being smaller than that of the concave surface; and
wherein the convex curved surface and the concave curved surface are configured such that when the first magnetic implant assembly and the second magnetic implant assembly are magnetically anastomosed, the convex curved surface protrudes towards the concave curved surface, and the axial distance between the convex curved surface and the concave curved surface along the central line direction of the first magnetic implant assembly increases as the radial distance from the central line of the first magnetic implant assembly increases, so as to exert the non-uniform compressive force on the first luminal tissue region and the second luminal tissue region.

19. The endoscopic magnetic anastomosis system according to claim 1, further comprising:
a magnet detector configured to be able to detect a position of the magnetic implant assembly of the endoscope within a human body.

20. The endoscopic magnetic anastomosis system according to claim 19, wherein the magnet detector comprises:
a detector body;
a first printed circuit board provided on a first end of the detector body and provided with a first group of magnetometers;
a second printed circuit board provided on a second end of the detector body opposite to the first end of the detector body, and provided with a second group of magnetometers; and
a processing unit configured to receive measurement data from the first group of magnetometers and the second group of magnetometers and to determine a position of the magnetic implant assembly of the endoscope in a human body based on the received data.

21. The endoscopic magnetic anastomosis system according to claim 20, wherein,
the first group of magnetometers comprises four magnetometers distributed in a square array, and a center of the square array coincides with a center of the first printed circuit board;
the second group of magnetometers comprises four magnetometers distributed in a square array, and a center of the square array coincides with a center of the second printed circuit board.

22. The endoscopic magnetic anastomosis system according to claim 19, wherein an IMU is embedded within the magnetic implant assembly of the endoscope, and wherein the magnet detector comprises:
a mounting substrate;
at least one sensor module, each sensor module comprises eight magnetic sensors, the eight magnetic sensors are regularly distributed in four rows and each row has two magnetic sensors, the two magnetic sensors are a first magnetic sensor and a second magnetic sensor, respectively, and two magnetic sensors in each row are in a misaligned arrangement with two magnetic sensors in an adjacent row; and
a processing module configured to be able to receive data from the sensor module and data of the IMU embedded within the magnetic implant assembly, and to determine a position of the magnetic implant assembly of the endoscope within a human body based on the received data.

23. The endoscopic magnetic anastomosis system according to claim 22, wherein the number of the at least one sensor module is between 1-32.

24. The endoscopic magnetic anastomosis system according to claim 1, further comprising:
a magnetic navigation console configured to move the magnetic implant assembly of the endoscope to a set position within a human body under action of a magnetic field.

25. The endoscopic magnetic anastomosis system according to claim 24, wherein the at least one endoscopic assembly comprises two endoscopic assemblies, and the magnetic navigation console comprises:
a mounting bracket comprising a movable first mounting arm and a movable second mounting arm;
a first magnetic actuator mounted to the first mounting arm and configured to enable the magnetic implant assembly of one of the two endoscope assemblies to move within a human body under action of a magnetic field; and
a second magnetic actuator mounted to the second mounting arm and configured to enable the magnetic implant assembly of the other of the two endoscope assemblies to move within a human body under action of a magnetic field, wherein the first magnetic actuator and the second magnetic actuator are configured to be able to move to a state where they overlap in a vertical direction so that magnetic implant assemblies of two endoscopes are able to move to a state where they overlap in a vertical direction within a human body.

26. The endoscopic magnetic anastomosis system according to claim 25, wherein the first magnetic actuator comprises a permanent magnet, and the permanent magnet of the first magnetic actuator comprises a conical section, and wherein a lower end cross-sectional dimension of the conical section is smaller than an upper end cross-sectional dimension of the conical section.

27. The endoscopic magnetic anastomosis system according to claim 26, wherein,
the permanent magnet of the first magnetic actuator further comprises a cylindrical section extending upward from the upper end of the conical section, and a cross-sectional dimension of the cylindrical section is equal to the upper end cross-sectional dimension of the conical section.

28. The endoscopic magnetic anastomosis system according to claim 25, wherein,
the second magnetic actuator comprises a permanent magnet; or
the second magnetic actuator comprises an electromagnetic coil; or
the second magnetic actuator comprises a permanent magnet and an electromagnetic coil disposed above or below the permanent magnet.

29. The endoscopic magnetic anastomosis system according to claim 25, wherein the first mounting arm is longitudinally slidably mounted to and hinged with the mounting bracket, the first mounting arm comprising a plurality of connecting arms hinged sequentially; and/or
the second mounting arm is longitudinally slidably mounted to the mounting bracket and hinged with the mounting bracket, the second mounting arm comprising a plurality of connecting arms hinged sequentially; and/or
wherein the second magnetic actuator is arranged so that an intensity of a magnetic field generated by the second magnetic actuator is greater than an intensity of a magnetic field generated by the first magnetic actuator, and the first magnetic actuator is positioned below the second magnetic actuator when the first magnetic actuator and the second magnetic actuator overlap in a vertical direction; and wherein at least one of an upper end of the first magnetic actuator and a lower end of the second magnetic actuator is provided with an anti-friction coating.

30. The endoscopic magnetic anastomosis system according to claim 25, wherein the magnetic navigation console further comprises:
a push rod mechanism configured to be able to extend and retract, and capable of pushing one of the first magnetic actuator and the second magnetic actuator to move the first magnetic actuator and the second magnetic actuator away from each other.

31. The endoscopic magnetic anastomosis system according to claim 1, wherein the at least one endoscopic assembly comprises two endoscope assemblies that are identical or different in structure.

32. An outer sheath comprising:
a body tube having a first end and an opposite second end; and
a tube-locking mechanism mounted at the first end of the body tube,
wherein the body tube further has an endoscopic channel extending from the first end of the body tube to the second end of the body tube for passage of an endoscope, the tube-locking mechanism is configured to be able to be in locking fit with the endoscope passing through the endoscopic channel to secure the endoscope with the body tube, and to disengage from being in locking fit with the endoscope passing through the endoscopic channel to enable the endoscope to slide and rotate relative to the body tube.

33. A magnet detector for detecting a position of a magnet, the magnet detector comprising:
a detector body;
a first printed circuit board provided on a first end of the detector body and provided with a first group of magnetometers;
a second printed circuit board provided on a second end of the detector body opposite to the first end, and provided with a second group of magnetometers; and
a processing unit configured to be able to receive measurement data from the first group of magnetometers and the second group of magnetometers and to determine a position of the magnet based on the received data.

34. A magnetic navigation console for driving a magnet to move under action of a magnetic field, the magnetic navigation console comprising:
a mounting bracket comprising a movable first mounting arm and a movable second mounting arm;
a first magnetic actuator mounted to the first mounting arm; and
a second magnetic actuator mounted to the second mounting arm, the first magnetic actuator and the second magnetic actuator are configured to be able to move to overlap in a vertical direction.
